SEQUENCE LISTING (33 sequences)

SEQ ID NO: 1 - protein, 5 aa, synthetic construct
SYAMN

SEQ ID NO: 2 - protein, 17 aa, synthetic construct
GISSSGRYTGYADSVKG

SEQ ID NO: 3 - protein, 18 aa, synthetic construct
SVGSGVSWSGYVATSLDA

SEQ ID NO: 4 - protein, 8 aa, synthetic construct
SAGSGLYG

SEQ ID NO: 5 - protein, 7 aa, synthetic construct
GTNKRPS

SEQ ID NO: 6 - protein, 11 aa, synthetic construct
GSADSSTNAGI

SEQ ID NO: 7 - protein, 127 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSS

SEQ ID NO: 8 - protein, 105 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVL

SEQ ID NO: 9 - protein, 5 aa, synthetic construct
TYAMN

SEQ ID NO: 10 - protein, 19 aa, synthetic construct
RIRSKYNNYATYYADSVKG

SEQ ID NO: 11 - protein, 14 aa, synthetic construct
HGNFGDSYVSWFAY

SEQ ID NO: 12 - protein, 14 aa, synthetic construct
GSSTGAVTTSNYAN

SEQ ID NO: 13 - protein, 7 aa, synthetic construct
GTNKRAP

SEQ ID NO: 14 - protein, 9 aa, synthetic construct
ALWYSNHWV

SEQ ID NO: 15 - protein, 125 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 16 - protein, 109 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 17 - protein, 457 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 211 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 19 - protein, 487 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLASPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 20 aa, synthetic construct
GKPGSGKPGSGKPGSGKPGS

SEQ ID NO: 21 - protein, 7 aa, synthetic construct
GFTFSSY

SEQ ID NO: 22 - protein, 6 aa, synthetic construct
SSSGRY

SEQ ID NO: 23 - protein, 8 aa, synthetic construct
GFTFSSYA

SEQ ID NO: 24 - protein, 8 aa, synthetic construct
ISSSGRYT

SEQ ID NO: 25 - protein, 20 aa, synthetic construct
AKSVGSGVSWSGYVATSLDA

SEQ ID NO: 26 - protein, 7 aa, synthetic construct
GFTFSTY

SEQ ID NO: 27 - protein, 8 aa, synthetic construct
RSKYNNYA

SEQ ID NO: 28 - protein, 8 aa, synthetic construct
GFTFSTYA

SEQ ID NO: 29 - protein, 10 aa, synthetic construct
IRSKYNNYAT

SEQ ID NO: 30 - protein, 16 aa, synthetic construct
VRHGNFGDSYVSWFAY

SEQ ID NO: 31 - protein, 9 aa, synthetic construct
TGAVTTSNY

SEQ ID NO: 32 - protein, 254 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 33 - protein, 233 aa, synthetic construct
ASPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

(19) United States
(12) Patent Application Publication
(10) Pub. No.: US 2025/0332255 A1
(43) Pub. Date: Oct. 30, 2025

(54) COMPOSITIONS COMPRISING BISPECIFIC ANTIBODIES THAT BIND TO CLAUDIN 6 AND CD3, AND USES THEREOF

(71) Applicant: Context Therapeutics Inc., Philadelphia, PA (US)

(72) Inventors: Edward Calamai, Kingston, NY (US); Eric A. Butz, Seattle, WA (US); Srijib Banerjee, Munich (DE); Elena Menchi, Basel (CH)

(21) Appl. No.: 19/192,838

(22) Filed: Apr. 29, 2025

Related U.S. Application Data
(60) Provisional application No. 63/640,643, filed on Apr. 30, 2024.

(51) Int. Cl. A61K 39/395; A61K 47/22; A61K 47/26; C07K 16/28

(52) U.S. Cl. CPC A61K 39/39591; A61K 47/22; A61K 47/26; C07K 16/28; C07K 16/2809; C07K 2317/31

(57) ABSTRACT

Provided for herein are pharmaceutical compositions comprising an antibody, such as a bispecific antibody that binds to Claudin 6 and CD3, and uses thereof, and methods of using the same.

### CROSS-REFERENCE TO RELATED APPLICATIONS

This application claims priority to U.S. Provisional Application No. 63/640,643, filed Apr. 30, 2024, which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Apr. 29, 2025, is named “CXH-021US_SL” and is 31,550 bytes in size.

### FIELD OF THE DISCLOSURE

The present disclosure is directed generally to compositions, such as pharmaceutical compositions, comprising bispecific antibodies that bind Claudin 6 (CLDN6) and CD3, and methods of use thereof.

### BACKGROUND

Cell adhesion proteins are critical for maintaining tissue integrity, as well as regulating diverse cellular events in a wide variety of physiological and pathological processes. Among cell adhesion proteins, some members of the claudin (CLDN) family are often aberrantly expressed in various cancers. Clinical application of CLDN therapeutics has been difficult because of lack of antibody specificity for particular CLDN proteins and widespread expression of closely related CLDN family members on normal cells. Thus, there remains a significant need for improved compositions and methods that can modulate the activity of CLDN family members to treat various cancers and diseases.

### BRIEF SUMMARY

In some embodiments, a pharmaceutical composition is provided comprising a bispecific antibody comprising a CLDN6 binding domain and a CD3 binding domain; a pharmaceutically acceptable buffer at a concentration of about 5 mM to about 100 mM; a sugar at a concentration of about 50 mM to about 500 mM; and a non-ionic surfactant in an amount of about 0.003% (w/v) to about 0.3% (w/v).

In some embodiments, the CLDN6 binding domain comprises a first variable heavy chain region (VH) and a first variable light chain region (VL), wherein the first VH comprises a HCDR1 of SEQ ID NO: 1, a HCDR2 of SEQ ID NO: 2, and a HCDR3 of SEQ ID NO: 3, and the first VL comprises a LCDR1 of SEQ ID NO: 4, a LCDR2 of SEQ ID NO: 5, and a LCDR3 of SEQ ID NO: 6.

In some embodiments, the CLDN6 binding domain comprises a first VH comprising an amino acid sequence having at least 90% identity to the amino acid sequence of SEQ ID NO: 7, and comprises a first VL comprising an amino acid sequence having at least 90% identity to the amino acid sequence of SEQ ID NO: 8.

In some embodiments, the CLDN6 binding domain comprises a heavy chain (HC) and a light chain (LC), wherein the HC comprises an amino acid sequence having at least 90% identity to the amino acid sequence of SEQ ID NO: 17 and the LC comprises an amino acid sequence having at least 90% identity to the amino acid sequence of SEQ ID NO: 18.

In some embodiments, the bispecific antibody comprising a CLDN6 binding domain and a CD3 binding domain comprises: a first polypeptide comprising a first light chain comprising a first variable light chain region, wherein the first variable light chain region comprises: (1) a CDR1 sequence comprising the amino acid sequence of SEQ ID NO: 4; (2) a CDR2 sequence comprising the amino acid sequence of SEQ ID NO: 5; and (3) a CDR3 sequence comprising the amino acid sequence of SEQ ID NO: 6; a second polypeptide comprising a first heavy chain comprising a first variable heavy chain region and a constant domain, wherein the first variable heavy chain region comprises: (1) a CDR1 comprising the amino acid sequence of SEQ ID NO: 1; (2) a CDR2 comprising the amino acid sequence of SEQ ID NO: 2, and (3) a CDR3 comprising the amino acid sequence of SEQ ID NO: 3; and a third polypeptide comprising a second light chain and a second heavy chain and a constant domain, wherein: the second heavy chain comprises a second variable heavy chain region comprising: (1) a CDR1 comprising the amino acid sequence of SEQ ID NO: 9; (2) a CDR2 comprising the amino acid sequence of SEQ ID NO: 10; and (3) a CDR3 comprising the amino acid sequence of SEQ ID NO: 11; and the second light chain comprises a second variable light chain region comprising: (1) a CDR1 comprising the amino sequence of SEQ ID NO: 12; (2) a CDR2 comprising the amino acid sequence of SEQ ID NO: 13; and (3) a CDR3 comprising the amino acid sequence of SEQ ID NO: 14, or a variant thereof, wherein: the first polypeptide comprises an amino acid sequence that is at least 95% identical to the amino acid sequence of SEQ ID NO: 18; the second polypeptide comprises an amino acid sequence that is at least 95% identical to the amino acid sequence of SEQ ID NO: 17; and the third polypeptide comprises an amino acid sequence that is at least 95% identical to the amino acid sequence of SEQ ID NO: 19.

In some embodiments, a pharmaceutical composition is provided comprising: a bispecific antibody comprising a CLDN6 binding domain and a CD3 binding domain in an amount of about 1 mg/mL to about 50 mg/mL; a histidine buffer at a concentration of about 5 mM to about 100 mM; sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0.

In some embodiments, a pharmaceutical composition is provided comprising a bispecific antibody comprising a CLDN6 binding domain and a CD3 binding domain in an amount of about 5 mg/mL to about 30 mg/mL; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5.

In some embodiments, a pharmaceutical composition is provided comprising a bispecific antibody comprising a CLDN6 binding domain and a CD3 binding domain in an amount of about 10 mg/mL to about 20 mg/mL; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5.

In some embodiments, a pharmaceutical composition is provided comprising a bispecific antibody comprising a CLDN6 binding domain and a CD3 binding domain in an amount of about 15 mg/mL; a histidine buffer at a concentration of about 20 mM; sucrose at a concentration of about 240 mM; a polysorbate-20 in an amount of about 0.03% (w/v); and wherein the pharmaceutical composition is at a pH of about 6.0.

In some embodiments, a pharmaceutical composition is provided comprising a bispecific antibody comprising a CLDN6 binding domain and a CD3 binding domain in an amount of about 1 mg/mL to about 50 mg/mL, wherein the bispecific antibody is a scFv-Fab-Fc; a histidine buffer at a concentration of about 5 mM to about 100 mM; sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0.

In some embodiments, a pharmaceutical composition is provided comprising a bispecific antibody comprising a CLDN6 binding domain and a CD3 binding domain in an amount of about 5 mg/mL to about 30 mg/mL, wherein the bispecific antibody is a scFv-Fab-Fc; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5.

In some embodiments, a pharmaceutical composition is provided comprising a bispecific antibody comprising a CLDN6 binding domain and a CD3 binding domain in an amount of about 10 mg/mL to about 20 mg/mL, wherein the bispecific antibody is a scFv-Fab-Fc; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5.

In some embodiments, a pharmaceutical composition is provided comprising a bispecific antibody comprising a CLDN6 binding domain and a CD3 binding domain in an amount of about 15 mg/mL, wherein the bispecific antibody is a scFv-Fab-Fc; a histidine buffer at a concentration of about 20 mM; sucrose at a concentration of about 240 mM; a polysorbate-20 in an amount of about 0.03% (w/v); and wherein the pharmaceutical composition is at a pH of about 5.9-6.0.

In some embodiments, a pharmaceutical composition is provided comprising a bispecific antibody comprising a CLDN6 binding domain and a CD3 binding domain in an amount of about 1 mg/mL to about 50 mg/mL; a histidine buffer at a concentration of about 5 mM to about 100 mM; sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a first VH and a first VL, wherein the first VH comprises a HCDR1 of SEQ ID NO: 1, a HCDR2 of SEQ ID NO: 2, and a HCDR3 of SEQ ID NO: 3, and the first VL comprises a LCDR1 of SEQ ID NO: 4, a LCDR2 of SEQ ID NO: 5, and a LCDR3 of SEQ ID NO: 6; and wherein the CD3 binding domain comprises a second VH and a second VL, wherein the second VH comprises a HCDR1 of SEQ ID NO: 9, a HCDR2 of SEQ ID NO: 10, and a HCDR3 of SEQ ID NO: 11, and the second VL comprises a LCDR1 of SEQ ID NO: 12, a LCDR2 of SEQ ID NO: 13, and a LCDR3 of SEQ ID NO: 14.

In some embodiments, a pharmaceutical composition is provided comprising a bispecific antibody comprising a CLDN6 binding domain and a CD3 binding domain in an amount of about 5 mg/mL to about 30 mg/mL; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a first VH and a first VL, wherein the first VH comprises a HCDR1 of SEQ ID NO: 1, a HCDR2 of SEQ ID NO: 2, and a HCDR3 of SEQ ID NO: 3, and the first VL comprises a LCDR1 of SEQ ID NO: 4, a LCDR2 of SEQ ID NO: 5, and a LCDR3 of SEQ ID NO: 6; and wherein the CD3 binding domain comprises a second VH and a second VL, wherein the second VH comprises a HCDR1 of SEQ ID NO: 9, a HCDR2 of SEQ ID NO: 10, and a HCDR3 of SEQ ID NO: 11, and the second VL comprises a LCDR1 of SEQ ID NO: 12, a LCDR2 of SEQ ID NO: 13, and a LCDR3 of SEQ ID NO: 14.

In some embodiments, a pharmaceutical composition is provided comprising a bispecific antibody comprising a CLDN6 binding domain and a CD3 binding domain in an amount of about 10 mg/mL to about 20 mg/mL; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a first VH and a first VL, wherein the first VH comprises a HCDR1 of SEQ ID NO: 1, a HCDR2 of SEQ ID NO: 2, and a HCDR3 of SEQ ID NO: 3, and the first VL comprises a LCDR1 of SEQ ID NO: 4, a LCDR2 of SEQ ID NO: 5, and a LCDR3 of SEQ ID NO: 6; and wherein the CD3 binding domain comprises a second VH and a second VL, wherein the second VH comprises a HCDR1 of SEQ ID NO: 9, a HCDR2 of SEQ ID NO: 10, and a HCDR3 of SEQ ID NO: 11, and the second VL comprises a LCDR1 of SEQ ID NO: 12, a LCDR2 of SEQ ID NO: 13, and a LCDR3 of SEQ ID NO: 14.

In some embodiments, a pharmaceutical composition is provided comprising a bispecific antibody comprising a CLDN6 binding domain and a CD3 binding domain in an amount of about 15 mg/mL; a histidine buffer at a concentration of about 20 mM; sucrose at a concentration of about 240 mM; a polysorbate-20 in an amount of about 0.03% (w/v); and wherein the pharmaceutical composition is at a pH of about 5.9-6.1; wherein the CLDN6 binding domain comprises a first VH and a first VL, wherein the first VH comprises a HCDR1 of SEQ ID NO: 1, a HCDR2 of SEQ ID NO: 2, and a HCDR3 of SEQ ID NO: 3, and the first VL comprises a LCDR1 of SEQ ID NO: 4, a LCDR2 of SEQ ID NO: 5, and a LCDR3 of SEQ ID NO: 6; and wherein the CD3 binding domain comprises a second VH and a second VL, wherein the second VH comprises a HCDR1 of SEQ ID NO: 9, a HCDR2 of SEQ ID NO: 10, and a HCDR3 of SEQ ID NO: 11, and the second VL comprises a LCDR1 of SEQ ID NO: 12, a LCDR2 of SEQ ID NO: 13, and a LCDR3 of SEQ ID NO: 14.

In some embodiments, a pharmaceutical composition is provided comprising a bispecific antibody comprising a CLDN6 binding domain and a CD3 binding domain in an amount of about 10 mg/mL to about 20 mg/mL; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the bispecific antibody comprising a CLDN6 binding domain and a CD3 binding domain comprises: a first polypeptide comprising a first light chain comprising a first variable light chain region, wherein the first variable light chain region comprises: (1) a CDR1 sequence comprising the amino acid sequence of SEQ ID NO: 4; (2) a CDR2 sequence comprising the amino acid sequence of SEQ ID NO: 5; and (3) a CDR3 sequence comprising the amino acid sequence of SEQ ID NO: 6; a second polypeptide comprising a first heavy chain comprising a first variable heavy chain region and a constant domain, wherein the first variable heavy chain region comprises: (1) a CDR1 comprising the amino acid sequence of SEQ ID NO: 1; (2) a CDR2 comprising the amino acid sequence of SEQ ID NO: 2, and (3) a CDR3 comprising the amino acid sequence of SEQ ID NO: 3; and a third polypeptide comprising a second light chain and a second heavy chain and a constant domain, wherein: the second heavy chain comprises a second variable heavy chain region comprising: (1) a CDR1 comprising the amino acid sequence of SEQ ID NO: 9; (2) a CDR2 comprising the amino acid sequence of SEQ ID NO: 10; and (3) a CDR3 comprising the amino acid sequence of SEQ ID NO: 11; and the second light chain comprises a second variable light chain region comprising: (1) a CDR1 comprising the amino sequence of SEQ ID NO: 12; (2) a CDR2 comprising the amino acid sequence of SEQ ID NO: 13; and (3) a CDR3 comprising the amino acid sequence of SEQ ID NO: 14, or a variant thereof, wherein: the first polypeptide comprises an amino acid sequence that is at least 95% identical to the amino acid sequence of SEQ ID NO: 18; the second polypeptide comprises an amino acid sequence that is at least 95% identical to the amino acid sequence of SEQ ID NO: 17; and the third polypeptide comprises an amino acid sequence that is at least 95% identical to the amino acid sequence of SEQ ID NO: 19.

In some embodiments, a pharmaceutical composition is provided comprising a bispecific antibody comprising a CLDN6 binding domain and a CD3 binding domain in an amount of about 10 mg/mL to about 20 mg/mL; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the bispecific antibody comprising a CLDN6 binding domain and a CD3 binding domain comprises: a first polypeptide comprising the amino acid sequence of SEQ ID NO: 18; a second polypeptide comprising the amino acid sequence of SEQ ID NO: 17; and a third polypeptide comprising the amino acid sequence of SEQ ID NO: 19.

In some embodiments, a pharmaceutical composition is provided comprising a bispecific antibody comprising a CLDN6 binding domain and a CD3 binding domain in an amount of about 15 mg/mL; a histidine buffer at a concentration of about 20 mM; sucrose at a concentration of about 240 mM; a polysorbate-20 in an amount of about 0.03% (w/v); and wherein the pharmaceutical composition is at a pH of about 5.9-6.1; wherein the bispecific antibody comprising a CLDN6 binding domain and a CD3 binding domain comprises: a first polypeptide comprising a first light chain comprising a first variable light chain region, wherein the first variable light chain region comprises: (1) a CDR1 sequence comprising the amino acid sequence of SEQ ID NO: 4; (2) a CDR2 sequence comprising the amino acid sequence of SEQ ID NO: 5; and (3) a CDR3 sequence comprising the amino acid sequence of SEQ ID NO: 6; a second polypeptide comprising a first heavy chain comprising a first variable heavy chain region and a constant domain, wherein the first variable heavy chain region comprises: (1) a CDR1 comprising the amino acid sequence of SEQ ID NO: 1; (2) a CDR2 comprising the amino acid sequence of SEQ ID NO: 2, and (3) a CDR3 comprising the amino acid sequence of SEQ ID NO: 3; and a third polypeptide comprising a second light chain and a second heavy chain and a constant domain, wherein: the second heavy chain comprises a second variable heavy chain region comprising: (1) a CDR1 comprising the amino acid sequence of SEQ ID NO: 9; (2) a CDR2 comprising the amino acid sequence of SEQ ID NO: 10; and (3) a CDR3 comprising the amino acid sequence of SEQ ID NO: 11; and the second light chain comprises a second variable light chain region comprising: (1) a CDR1 comprising the amino sequence of SEQ ID NO: 12; (2) a CDR2 comprising the amino acid sequence of SEQ ID NO: 13; and (3) a CDR3 comprising the amino acid sequence of SEQ ID NO: 14, or a variant thereof, wherein: the first polypeptide comprises an amino acid sequence that is at least 95% identical to the amino acid sequence of SEQ ID NO: 18; the second polypeptide comprises an amino acid sequence that is at least 95% identical to the amino acid sequence of SEQ ID NO: 17; and the third polypeptide comprises an amino acid sequence that is at least 95% identical to the amino acid sequence of SEQ ID NO: 19.

In some embodiments, a pharmaceutical composition is provided comprising a bispecific antibody comprising a CLDN6 binding domain and a CD3 binding domain in an amount of about 15 mg/mL; a histidine buffer at a concentration of about 20 mM; sucrose at a concentration of about 240 mM; a polysorbate-20 in an amount of about 0.03% (w/v); and wherein the pharmaceutical composition is at a pH of about 5.9-6.1; wherein the bispecific antibody comprising a CLDN6 binding domain and a CD3 binding domain comprises: a first polypeptide comprising the amino acid sequence of SEQ ID NO: 18; a second polypeptide comprising the amino acid sequence of SEQ ID NO: 17; and a third polypeptide comprising the amino acid sequence of SEQ ID NO: 19.

In some embodiments, a method of treating a disease or disorder in a subject is provided, the method comprising administering to the subject a pharmaceutical composition as described herein to the subject thereby treating the disease or disorder.

In some embodiments, the disease or disorder is a cancer.

In some embodiments, the cancer is non-small cell lung cancer (NSCLC), ovarian cancer, gastric cancer, breast cancer, endometrial cancer, or testicular cancer.

In some embodiments, a pharmaceutical composition as described herein is provided for use in the treatment of a disease or disorder.

In some embodiments, use of a pharmaceutical composition as described herein is provided for the manufacture of a medicament for treatment of a disease or disorder.

### BRIEF DESCRIPTION OF THE DRAWINGS

FIG. 1A-E illustrate exemplary bispecific antibody configurations as provided for herein. FIG. 1A illustrates a tandem scFv format. FIG. 1B illustrates a scFv-Fab-Fc format. FIG. 1C illustrates an IgG-(scFv)₂ format. FIG. 1D and FIG. 1E illustrate two different IgG-scFv formats.

FIG. 2 illustrates the results of stability analysis for various formulation embodiments provided for herein.

### DETAILED DESCRIPTION

It is to be understood that the embodiments described herein are not limited to particular formulations, compositions and experimental conditions disclosed, as such formulations, compositions, and experimental conditions may vary. It is also to be understood that the terminology used herein is only for the purpose of describing particular embodiments, and it is not intended to be limiting.

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art.

Unless otherwise required by context, singular terms shall include pluralities, and plural terms shall include the singular.

That the disclosure may be more readily understood, select terms are defined below.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments. Additionally, where a phrase recites “about x to y,” the term “about” modifies both x and y and can be used interchangeably with the phrase “about x to about y” unless context dictates differently. Additionally, it should be understood that the values being modified by the term “about” are also provided without being modified by the term “about” but to avoid repetition in the present disclosure, the same value is not repeated without the term “about.” For example, the phrase “a pH of about 5.9 to about 6.1” also includes the range “a pH of 5.9 to 6.1.”

As used herein, the term “individual” or “subject,” or “patient” used interchangeably, means any animal, including mammals, such as mice, rats, other rodents, rabbits, dogs, cats, swine, cattle, sheep, horses, or primates, such as humans.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists.”

The term “antibody” as used herein refers to a protein which interacts with an antigen and comprises at least two heavy chains (HC) and two light chains (LC) inter-connected by disulfide bonds. Each heavy chain is comprised of a heavy chain variable region (VH) and a heavy chain constant region. The heavy chain constant region of an IgG is comprised of three domains, CH1, CH2 and CH3. Each light chain is comprised of a light chain variable region (VL) and a light chain constant region. The light chain constant region is comprised of one domain, CL. The VH and VL regions can be further subdivided into hypervariable regions, termed complementarity determining regions (CDR), interspersed with regions that are more conserved, termed framework regions (FR). Each VH and VL is composed of three CDRs and four FRs arranged from N-terminus to C-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes for example, monoclonal antibodies, human antibodies, humanized antibodies, camelized antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

As used herein, the term “antibody fragment” refers to one or more portions of an antibody that retain the ability to specifically interact (e.g., by binding, steric hindrance, stabilizing spatial distribution) with an antigen. Examples of binding fragments include, but are not limited to, a Fab fragment, a monovalent fragment consisting of the VL, VH, CL and CH1 domains; a F(ab′)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment, which consists of a VH domain; and an isolated CDR. Furthermore, although the two domains of the Fv fragment, VL and VH, are coded for by separate genes, they can be joined using recombinant methods by a synthetic linker that allows them to be made as a single protein chain in which the VL and VH regions pair to form monovalent molecules (known as single chain Fv (scFv). Such single chain antibodies are also intended to be encompassed within the term “antibody fragment.” These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR, and bis-scFv. Antibody fragments can be incorporated into single chain molecules comprising a pair of tandem Fv segments (VH-CH1-VH-CH1) which, together with complementary light chain polypeptides, form a pair of antigen-binding sites.

As used herein, the terms “complementarity-determining regions,” “CDRs,” and “hypervariable regions” refer to the parts of the variable domains in antibodies that determine the antibodies' binding specificities to their specific antigen. A single variable region of an antibody polypeptide will typically comprise three CDRs, usually designated CDR1, CDR2, and CDR3. More particularly, a heavy chain variable region may contain CDRs designated HCDR1, HCDR2, and HCDR3; likewise, light chain variable region may contain CDRs LCDR1, LCDR2, and LCDR3. Multiple methods may be used to define a CDR sequence. The current art utilizes various numbering schemes with different definitions of CDR lengths and positions. For example, IMGT numbering scheme is a standardized numbering system based on alignments of sequences from a complete reference gene database including the whole immunoglobulin superfamily. The Kabat numbering scheme is based on sequence alignment and uses “variability parameter” of a given amino acid position (the number of different amino acids at a given position divided by the frequency of the most occurring amino acid at that position) to predict CDRs. The Chothia numbering scheme, on the other hand, is a structure-based numbering scheme where antibody crystal structures are aligned as define the loop structures as CDRs. The Martin numbering scheme focuses on the structure alignment of different framework regions of unconventional lengths. Honneger's numbering scheme (Aho's) is based on structural alignments of the 3D structure of the variable regions and uses structurally conserved Ca positions to deduce framework and CDR lengths. One of skill in the art will note that the definition of a CDR will vary based on the method used. Accordingly, CDR sequences of a given heavy or light chain variable region may vary depending on the numbering system used. Any method of defining a CDR is contemplated with the sequences disclosed herein.

The terms “pharmaceutical formulation,” “pharmaceutical composition,” or “formulation” can be used interchangeably and refer to a preparation which is in such form as to permit the biological activity of an active pharmaceutical ingredient (API) contained therein to be effective and suitably stable for the purposes of storage and distribution, and which contains no additional components which are unacceptably toxic to a subject to which the formulation is administered. Such formulations are sterile. A “sterile” formulation is aseptic or free from all living microorganisms and their spores.

As used herein, the terms “treatment,” “treating,” and the like, in some cases, refer to administering an agent, or carrying out a procedure, for the purposes of obtaining an effect. The effect may be prophylactic in terms of completely or partially preventing a disease or symptom thereof and/or is therapeutic in terms of effecting a partial or complete cure for a disease and/or symptoms of the disease. “Treatment,” as used herein, includes treatment of a disease or disorder in a mammal, particularly in a human, and includes: (a) preventing the disease or a symptom of a disease from occurring in a subject which is predisposed to the disease but has not yet been diagnosed as having it (e.g., including diseases that is associated with or caused by a primary disease; (b) inhibiting the disease, i.e., arresting its development; and (c) relieving the disease, i.e., causing regression of the disease. The term treating includes to any indicia of success in the treatment or amelioration or prevention of a disease or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters, including the results of an examination by a physician. Accordingly, the term “treating” includes the administration of the agents of the present disclosure to prevent or delay, to alleviate, or to arrest or inhibit development of the symptoms or conditions associated with diseases. The term “therapeutic effect” refers to the reduction, elimination, or prevention of the disease, symptoms of the disease, or side effects of the disease in the subject. A subject is “treated” for a disease or disorder if, after receiving a therapeutic amount of an antibody of the present disclosure, the patient shows observable and/or measurable change in a parameter or symptom of the disease or disorder.

### Pharmaceutical Compositions

The term “composition” as used herein means a product which results from the mixing or combining of more than one element or ingredient.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer.

The phrase “pharmaceutically acceptable” is employed herein to refer to those agents of interest/compounds, salts, compositions, pharmaceutical dosage forms, etc., which are, within the scope of sound medical judgment, suitable for use in contact with the tissues of human beings and/or other mammals without excessive toxicity, irritation, allergic response, or other problem or complication, commensurate with a reasonable benefit/risk ratio. In some embodiments, pharmaceutically acceptable means approved by a regulatory agency of the federal or a state government, or listed in the U.S. Pharmacopeia or other generally recognized pharmacopeia for use in animals (e.g., mammals), and more particularly, in humans.

The term “excipient” refers to a pharmacologically inactive substance formulated with an antibody, antigen-binding fragment thereof, viral vector, or any other pharmacologically active molecules as provided for herein.

In some embodiments, a pharmaceutical composition is provided, the pharmaceutical composition comprising a bispecific antibody that binds to claudin 6 (CLDN6) and CD3, a pharmaceutically acceptable buffer, a sugar, and a non-ionic surfactant. Other embodiments of such compositions are provided for herein or are apparent from the present disclosure.

### Exemplary Bispecific Antibodies

In some embodiments, the bispecific antibody as provided for herein comprises a claudin 6 (CLDN6) binding domain and a CD3 binding domain. In some embodiments, the format of the bispecific antibody is as provided for herein. In some embodiments, the format of the bispecific antibody is a tandem scFv antibody, a scFv-Fab-Fc antibody, an IgG-(scFv)₂ antibody, or an IgG-(scFv)₁ antibody. In some embodiments, the bispecific antibody comprises a CLDN6 binding domain and a CD3 binding domain and is a tandem scFv antibody. In some embodiments, the bispecific antibody comprises a CLDN6 binding domain and a CD3 binding domain and is a tandem scFv-Fab-Fc antibody. In some embodiments, the bispecific antibody comprises a CLDN6 binding domain and a CD3 binding domain and is an IgG-(scFv)₂ antibody. In some embodiments, the bispecific antibody comprises a CLDN6 binding domain and a CD3 binding domain and is an IgG-(scFv)₁ antibody.

In some embodiments, the bispecific antibody comprises a CLDN6 binding domain and a CD3 binding domain, wherein the CLDN6 binding domain comprises a first variable heavy chain region (V_{H}) and a first variable light chain region (V_{L}) and wherein the CD3 binding domain comprises a second variable heavy chain region (V_{H}) and a second variable light chain region (V_{L}). In some embodiments, the bispecific antibody comprises a CLDN6 binding domain and a CD3 binding domain, wherein the CLDN6 binding domain comprises a first V_{H} comprising a HCDR1, HCDR2, and HCDR3 as provided for in Tables 1, 2, or 3, and a first V_{L} comprising a LCDR1, LCDR2, and LCDR3 as provided for in Tables 1, 2, or 3; and wherein the CD3 binding domain comprises a second V_{H} comprising a HCDR1, HCDR2, and HCDR3 as provided for in Tables 4, 5, or 6 and a second V_{L} comprising a LCDR1, LCDR2, and LCDR3 as provided for in Tables 4, 5, or 6.

TABLE 1
CDRs of CLDN6 binding domain - Kabat numbering
Kabat CDRs

| Domain | HCDR1 | HCDR2 | HCDR3 | LCDR1 | LCDR2 | LCDR3 |
|---|---|---|---|---|---|---|
| CLDN6 | SYAMN (SEQ | GISSSGRYT | SVGSGVSWS | SAGSGLYG | GTNKRPS | GSADSSTNA |
| binding | ID NO: 1) | GYADSVKG | GYVATSLDA | (SEQ ID | (SEQ ID | GI (SEQ |
| domain | | (SEQ ID | (SEQ ID | NO: 4) | NO: 5) | ID NO: 6) |
| | | NO: 2) | NO: 3) | | | |

TABLE 2
CDRs of CLDN6 binding domain - Chothia numbering
Chothia CDRs

| Domain | HCDR1 | HCDR2 | HCDR3 | LCDR1 | LCDR2 | LCDR3 |
|---|---|---|---|---|---|---|
| CLDN6 | GFTFSSY | SSSGRY | SVGSGVSWS | SAGSGLYG | GTNKRPS | GSADSSTNA |
| binding | (SEQ ID | (SEQ ID | GYVATSLDA | (SEQ ID | (SEQ ID | GI (SEQ |
| domain | NO: 21) | NO: 22) | (SEQ ID | NO: 4) | NO: 5) | ID NO: 6) |
| | | | NO: 3) | | | |

TABLE 3
CDRs of CLDN6 binding domain - IMGT numbering
IMGT CDRs

| Domain | HCDR1 | HCDR2 | HCDR3 | LCDR1 | LCDR2 | LCDR3 |
|---|---|---|---|---|---|---|
| CLDN6 | GFTFSSYA | ISSSGRYT | AKSVGSGVS | SGL | GT | GSADSSTNA |
| binding | (SEQ ID | (SEQ ID | WSGYVATSL | (tripep- | (dipep- | GI (SEQ |
| domain | NO: 23) | NO: 24) | DA (SEQ | tide) | tide) | ID NO: 6) |
| | | | ID NO: 25) | | | |

TABLE 4
CDRs of CD3 binding domain - Kabat numbering
Kabat CDRs

| Domain | HCDR1 | HCDR2 | HCDR3 | LCDR1 | LCDR2 | LCDR3 |
|---|---|---|---|---|---|---|
| CD3 | TYAMN | RIRSKYNNYA | HGNFGDSY | GSSTGAVTT | GTNKRAP | ALWYSNHW |
| binding | (SEQ ID | TYYADSVKG | VSWFAY | SNYAN | (SEQ ID | V (SEQ |
| domain | NO: 9) | (SEQ ID | (SEQ ID | (SEQ ID | NO: 13) | ID NO: |
| | | NO: 10) | NO: 11) | NO: 12) | | 14) |

TABLE 5
CDRs of CD3 binding domain - Chothia numbering
Chothia CDRs

| Domain | HCDR1 | HCDR2 | HCDR3 | LCDR1 | LCDR2 | LCDR3 |
|---|---|---|---|---|---|---|
| CD3 | GFTFSTY | RSKYNNYA | HGNFGDSY | GSSTGAVTT | GTNKRAP | ALWYSNHW |
| binding | (SEQ ID | (SEQ ID | VSWFAY | SNYAN (SEQ | (SEQ ID | V (SEQ |
| domain | NO: 26) | NO: 27) | (SEQ ID | ID NO: 12) | NO: 13) | ID NO: |
| | | | NO: 11) | | | 14) |

TABLE 6
CDRs of CD3 binding domain - IMGT numbering
IMGT CDRs

| Domain | HCDR1 | HCDR2 | HCDR3 | LCDR1 | LCDR2 | LCDR3 |
|---|---|---|---|---|---|---|
| CD3 | GFTFSTYA | IRSKYNNYAT | VRHGNFGD | TGAVTTSNY | GT | ALWYSNHW |
| binding | (SEQ ID | (SEQ ID | SYVSWFAY | (SEQ ID | (dipep- | V (SEQ |
| domain | NO: 28) | NO: 29) | (SEQ ID | NO: 31) | tide) | ID NO: |
| | | | NO: 30) | | | 14) |

In some embodiments, the bispecific antibody comprises a CLDN6 binding domain and a CD3 binding domain, wherein the CLDN6 binding domain comprises (i) a first V_{H} comprising heavy chain CDR1, CDR2, and CDR3 sequences, wherein the heavy chain CDR1 sequence has the amino acid sequence of SEQ ID NO: 1; the heavy chain CDR2 sequence has the amino acid sequence of SEQ ID NO: 2; and the heavy chain CDR3 sequence has the amino acid sequence of SEQ ID NO: 3; and (ii) a first V_{L} comprising light chain CDR1, CDR2, and CDR3 sequences, wherein the light chain CDR1 sequence has the amino acid sequence of SEQ ID NO: 4; the light chain CDR2 sequence has the amino acid sequence of SEQ ID NO: 5; and the light chain CDR3 sequence has the amino acid sequence of SEQ ID NO: 6; and wherein the CD3 binding domain comprises (i) a second V_{H} comprising heavy chain CDR1, CDR2, and CDR3 sequences, wherein the heavy chain CDR1 sequence has the amino acid sequence of SEQ ID NO: 9; the heavy chain CDR2 sequence has the amino acid sequence of SEQ ID NO: 10; and the heavy chain CDR3 sequence has the amino acid sequence of SEQ ID NO: 11; and (ii) a second V_{L} comprising light chain CDR1, CDR2, and CDR3 sequences, wherein the light chain CDR1 sequence has the amino acid sequence of SEQ ID NO: 12; the light chain CDR2 sequence has the amino acid sequence of SEQ ID NO: 13; and the light chain CDR3 sequence has the amino acid sequence of SEQ ID NO: 14.

In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence substantially similar to as provided for in Table 7, (ii) the first V_{L} comprises an amino acid sequence substantially similar to as provided for in Table 7, (iii) the second V_{H} comprises an amino acid sequence substantially similar to as provided for in Table 8, and (iv) the second V_{L} comprises an amino acid sequence substantially similar to as provided for in Table 8.

TABLE 7
Sequences of the VH region and VL region of the CLDN6 binding domain

| Domain | SEQ ID NO | Sequence |
|---|---|---|
| V_{H} - CLDN6 | SEQ ID NO: 7 | EVQLLESGGGLVQPGGSLRLSCAASGFTFSSYAMNWVRQAPGKGLEWVAGIS |
| binding | | SSGRYTGYADSVKGRFTISRDNSKNTLYLQMNSLRAEDTAVYYCAKSVGSGV |
| domain | | SWSGYVATSLDAWGQGTLVTVSS |
| V_{L} - CLDN6 | SEQ ID NO: 8 | SYELTQPPSVSVSPGQTARITCSAGSGLYGWYQQKPGQAPVLVIYGTNKRPS |
| binding | | GIPERFSGSSSGTTVTLTISGVQAEDEADYYCGSADSSTNAGIFGGGTKLTV |
| domain | | L |

TABLE 8
Sequences of the VH region and VL region of the CD3 binding domain

| Domain | SEQ ID NO | Sequence |
|---|---|---|
| V_{H} - CD3 | SEQ ID NO: | EVQLVESGGGLVQPGGSLRLSCAASGFTFSTYAMNWVRQAPGKGLEWVGRI |
| binding | 15 | RSKYNNYATYYADSVKGRFTISRDDSKNTLYLQMNSLRAEDTAVYYCVRHG |
| domain | | NFGDSYVSWFAYWGQGTLVTVSS |
| V_{L} - CD3 | SEQ ID NO: | QAVVTQEPSLTVSPGGTVTLTCGSSTGAVTTSNYANWVQQKPGKSPRGLIG |
| binding | 16 | GTNKRAPGVPARFSGSLLGGKAALTISGAQPEDEADYYCALWYSNHWVFGG |
| domain | | GTKLTVL |

In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, (iii) the second V_{H} comprises an amino acid sequence having at least 90%, 91%, 92%, 93%, 94%, 95%, 96%, 97%, 98%, or 99% identity to the amino acid sequence of SEQ ID NO: 15 as provided for herein, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16 as provided for herein.

In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence having at least 95% identity to the amino acid sequence of SEQ ID NO: 7, (ii) the first V_{L} comprises an amino acid sequence having at least 95% identity to the amino acid sequence of SEQ ID NO: 8, (iii) the second V_{H} comprises an amino acid sequence having at least 95% identity to the amino acid sequence of SEQ ID NO: 15, and (iv) the second V_{L} comprises an amino acid sequence having at least 95% identity to the amino acid sequence of SEQ ID NO: 16. In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence having at least 98% identity to the amino acid sequence of SEQ ID NO: 7, (ii) the first V_{L} comprises an amino acid sequence having at least 98% identity to the amino acid sequence of SEQ ID NO: 8, (iii) the second V_{H} comprises an amino acid sequence having at least 98% identity to the amino acid sequence of SEQ ID NO: 15, and (iv) the second V_{L} comprises an amino acid sequence having at least 98% identity to the amino acid sequence of SEQ ID NO: 16. In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises the amino acid sequence of SEQ ID NO: 7, (ii) the first V_{L} comprises the amino acid sequence of SEQ ID NO: 8, (iii) the second V_{H} comprises the amino acid sequence of SEQ ID NO: 15, and (iv) the second V_{L} comprises the amino acid sequence of SEQ ID NO: 16.

In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7, provided that the first V_{H} comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, (ii) the first V_{L} comprises an amino acid sequence having at least 90%, 91%, 92%, 93%, 94%, 95%, 96%, 97%, 98%, or 99% identity to the amino acid sequence of SEQ ID NO: 8, provided that the first V_{L} comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, (iii) the second V_{H} comprises an amino acid sequence having at least 90%, 91%, 92%, 93%, 94%, 95%, 96%, 97%, 98%, or 99% identity to the amino acid sequence of SEQ ID NO: 15, provided that the second V_{H} comprises a HCDR1 comprising the amino acid sequence of SEQ ID NO: 9, a HCDR2 comprising the amino acid sequence of SEQ ID NO: 10, and a HCDR3 comprising the amino acid sequence of SEQ ID NO: 11, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16, provided that the second V_{L} comprises a LCDR1 comprising the amino acid sequence of SEQ ID NO: 12, a LCDR2 comprising the amino acid sequence of SEQ ID NO: 13, and a LCDR3 comprising the amino acid sequence of SEQ ID NO: 14.

In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence having at least 95% identity to the amino acid sequence of SEQ ID NO: 7, provided that the first V_{H} comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, (ii) the first V_{L} comprises an amino acid sequence having at least 95% identity to the amino acid sequence of SEQ ID NO: 8, provided that the first V_{L} comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, (iii) the second V_{H} comprises an amino acid sequence having at least 95% identity to the amino acid sequence of SEQ ID NO: 15, provided that the second V_{H} comprises a HCDR1 comprising the amino acid sequence of SEQ ID NO: 9, a HCDR2 comprising the amino acid sequence of SEQ ID NO: 10, and a HCDR3 comprising the amino acid sequence of SEQ ID NO: 11, and (iv) the second V_{L} comprises an amino acid sequence having at least 95% identity to the amino acid sequence of SEQ ID NO: 16, provided that the second V_{L} comprises a LCDR1 comprising the amino acid sequence of SEQ ID NO: 12, a LCDR2 comprising the amino acid sequence of SEQ ID NO: 13, and a LCDR3 comprising the amino acid sequence of SEQ ID NO: 14.

In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence having at least 98% identity to the amino acid sequence of SEQ ID NO: 7, provided that the first V_{H} comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, (ii) the first V_{L} comprises an amino acid sequence having at least 98% identity to the amino acid sequence of SEQ ID NO: 8, provided that the first V_{L} comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, (iii) the second V_{H} comprises an amino acid sequence having at least 98% identity to the amino acid sequence of SEQ ID NO: 15, provided that the second V_{H} comprises a HCDR1 comprising the amino acid sequence of SEQ ID NO: 9, a HCDR2 comprising the amino acid sequence of SEQ ID NO: 10, and a HCDR3 comprising the amino acid sequence of SEQ ID NO: 11, and (iv) the second V_{L} comprises an amino acid sequence having at least 98% identity to the amino acid sequence of SEQ ID NO: 16, provided that the second V_{L} comprises a LCDR1 comprising the amino acid sequence of SEQ ID NO: 12, a LCDR2 comprising the amino acid sequence of SEQ ID NO: 13, and a LCDR3 comprising the amino acid sequence of SEQ ID NO: 14.

In some embodiments, the bispecific antibody comprises a CLDN6 binding domain and a CD3 binding domain, wherein the CLDN6 binding domain comprises a heavy chain (HC) and a light chain (LC), the heavy chain comprising a first V_{H} as provided for in Table 7 and the LC comprising a first V_{L} as provided for in Table 7, and wherein the CD3 binding domain comprises a scFv comprising a second V_{H} as provided for in Table 8 and a second V_{L} as provided for in Table 8.

In some embodiments, the bispecific antibody comprises a CLDN6 binding domain and a CD3 binding domain, wherein the CLDN6 binding domain comprises a HC comprising an amino acid sequence substantially similar as provided for in Table 9 and a LC comprising an amino acid sequence substantially similar to as provided for in Table 9, and wherein the CD3 binding domain comprises a scFv comprising an amino acid sequence substantially similar to as provided for in Table 10.

TABLE 9
Sequences of the HC and LC of the CLDN6

| Domain | SEQ ID NO | Sequence |
|---|---|---|
| HC - | SEQ ID NO: | EVQLLESGGGLVQPGGSLRLSCAASGFTFSSYAMNWVRQAPGKGLEWVAGIS |
| CLDN6 | 17 | SSGRYTGYADSVKGRFTISRDNSKNTLYLQMNSLRAEDTAVYYCAKSVGSGV |
| | | SWSGYVATSLDAWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQTY |
| | | ICNVNHKPSNTKVDKKVEPKSCDKTHTCPPCPAPEAAGGPSVFLFPPKPKDT |
| | | LMISRTPEVTCVVVDVSHEDPEVKFNWYVDGVEVHNAKTKPREEQYNSTYRV |
| | | VSVLTVLHQDWLNGKEYKCKVSNKALPAPIEKTISKAKGQPREPQVYTLPPS |
| | | REEMTKNQVSLWCLVKGFYPSDIAVEWESNGQPENNYKTTPPVLDSDGSFFL |
| | | YSKLTVDKSRWQQGNVFSCSVMHEALHNHYTQKSLSLSPGK |
| LC - | SEQ ID NO: | SYELTQPPSVSVSPGQTARITCSAGSGLYGWYQQKPGQAPVLVIYGTNKRPS |
| CLDN6 | 18 | GIPERFSGSSSGTTVTLTISGVQAEDEADYYCGSADSSTNAGIFGGGTKLTV |
| | | LGQPKAAPSVTLFPPSSEELQANKATLVCLISDFYPGAVTVAWKADSSPVKA |
| | | GVETTTPSKQSNNKYAASSYLSLTPEQWKSHRSYSCQVTHEGSTVEKTVAPT |
| | | ECS |

TABLE 10
Sequence of CD3 binding domain in scFv format without a constant domain.

| Domain | SEQ ID NO | Sequence |
|---|---|---|
| CD3 binding | SEQ ID NO: | EVQLVESGGGLVQPGGSLRLSCAASGFTFSTYAMNWVRQAPGKGLEWVGRIR |
| domain scFv | 32 | SKYNNYATYYADSVKGRFTISRDDSKNTLYLQMNSLRAEDTAVYYCVRHGNF |
| | | GDSYVSWFAYWGQGTLVTVSSGKPGSGKPGSGKPGSGKPGSQAVVTQEPSLT |
| | | VSPGGTVTLTCGSSTGAVTTSNYANWVQQKPGKSPRGLIGGTNKRAPGVPAR |
| | | FSGSLLGGKAALTISGAQPEDEADYYCALWYSNHWVFGGGTKLTVL |

In some embodiments, the bispecific antibody comprises a CLDN6 binding domain and a CD3 binding domain, wherein the CLDN6 binding domain comprises a HC comprising an amino acid sequence having at least 90%, 91%, 92%, 93%, 94%, 95%, 96%, 97%, 98%, or 99% identity to the amino acid sequence of SEQ ID NO: 17 and a LC comprising an amino acid sequence having at least 90%, 91%, 92%, 93%, 94%, 95%, 96%, 97%, 98%, or 99% identity to the amino acid sequence of SEQ ID NO: 18, and wherein the CD3 binding domain comprises a scFv comprising an amino acid sequence having at least 90%, 91%, 92%, 93%, 94%, 95%, 96%, 97%, 98%, or 99% identity to the amino acid sequence of SEQ ID NO: 32.

In some embodiments, the bispecific antibody comprises a CLDN6 binding domain comprising a HC and a LC and a CD3 binding domain comprising an scFv, wherein (i) the HC comprises an amino acid sequence having at least 95% identity to the amino acid sequence of SEQ ID NO: 17, (ii) the LC comprises an amino acid sequence having at least 95% identity to the amino acid sequence of SEQ ID NO: 18, and (iii) the scFv comprises an amino acid sequence having at least 95% identity to the amino acid sequence of SEQ ID NO: 32. In some embodiments, the bispecific antibody comprises a CLDN6 binding domain comprising a HC and a LC and a CD3 binding domain comprising an scFv, wherein (i) the HC comprises an amino acid sequence having at least 98% identity to the amino acid sequence of SEQ ID NO: 17, (ii) the LC comprises an amino acid sequence having at least 98% identity to the amino acid sequence of SEQ ID NO: 18, and (iii) the scFv comprises an amino acid sequence having at least 98% identity to the amino acid sequence of SEQ ID NO: 32. In some embodiments, the bispecific antibody comprises a CLDN6 binding domain comprising a HC and a LC and a CD3 binding domain comprising an scFv, wherein (i) the HC comprises the amino acid sequence of SEQ ID NO: 17, (ii) the LC comprises the amino acid sequence of SEQ ID NO: 18, and (iii) the scFv comprises the amino acid sequence of SEQ ID NO: 32.

In some embodiments, the V_{H} and the V_{L} of the scFv are linked directly to one another. In some embodiments, the V_{H} and the V_{L} are linked indirectly to one another, such as through a polypeptide linker. Polypeptide linkers are known in the art and any such polypeptide linker is within the scope of the present disclosure. In some embodiments, the polypeptide linker comprises the amino acid sequence of GKPGSGKPGSGKPGSGKPGS (SEQ ID NO: 20).

In some embodiments, the bispecific antibody comprises a CLDN6 binding domain and a CD3 binding domain, wherein the CLDN6 binding domain comprises a heavy chain (HC) and a light chain (LC), the heavy chain comprising a first V_{H} with an amino acid sequence substantially similar as to an amino acid sequence as provided for in Table 7 and the LC comprising a first V_{L} with an amino acid sequence substantially similar as to an amino acid sequence as provided for Table 7, and wherein the CD3 binding domain comprises a scFv comprising a second V_{H} with an amino acid sequence substantially similar as to an amino acid sequence as provided for in Table 8 and a second V_{L} with an amino acid sequence substantially similar as to an amino acid sequence as provided for in Table 8, and wherein the CD3 binding domain further comprises a constant domain with an amino acid sequence substantially similar as to an amino acid sequence as provided for in Table 11.

TABLE 11
Sequences of the scFv and constant region of an exemplary
CD3 binding domain

| Domain | SEQ ID NO | Sequence |
|---|---|---|
| CD3 binding | SEQ ID NO: | EVQLVESGGGLVQPGGSLRLSCAASGFTFSTYAMNWVRQAPGKGLEWVGRIR |
| domain scFv + | 19 | SKYNNYATYYADSVKGRFTISRDDSKNTLYLQMNSLRAEDTAVYYCVRHGNF |
| constant | | GDSYVSWFAYWGQGTLVTVSSGKPGSGKPGSGKPGSGKPGSQAVVTQEPSLT |
| | | VSPGGTVTLTCGSSTGAVTTSNYANWVQQKPGKSPRGLIGGTNKRAPGVPAR |
| | | FSGSLLGGKAALTISGAQPEDEADYYCALWYSNHWVFGGGTKLTVLASPKSS |
| | | DKTHTCPPCPAPEAAGGPSVFLFPPKPKDTLMISRTPEVTCVVVDVSHEDPE |
| | | VKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLSCAVKGFYPSD |
| | | IAVEWESNGQPENNYKTTPPVLDSDGSFFLVSKLTVDKSRWQQGNVFSCSVM |
| | | HEALHNHYTQKSLSLSPGK |

In some embodiments, the bispecific antibody comprises a CLDN6 binding domain and a CD3 binding domain, wherein the CLDN6 binding domain comprises a HC comprising an amino acid sequence substantially similar to the amino acid sequence as provided for in Table 9 and a LC comprising an amino acid sequence substantially similar to the amino acid sequence as provided for in Table 9, and wherein the CD3 binding domain comprises a scFv and a constant domain and comprises an amino acid sequence substantially similar to the amino acid sequence as provided for in Table 11.

In some embodiments, the bispecific antibody comprises a CLDN6 binding domain and a CD3 binding domain, wherein the CLDN6 binding domain comprises a HC comprising an amino acid sequence having at least 90%, 91%, 92%, 93%, 94%, 95%, 96%, 97%, 98%, or 99% identity to the amino acid sequence of SEQ ID NO: 17 and a LC comprising an amino acid sequence having at least 90%, 91%, 92%, 93%, 94%, 95%, 96%, 97%, 98%, or 99% identity to the amino acid sequence of SEQ ID NO: 18, and wherein the CD3 binding domain comprises a scFv and a constant domain and comprises an amino acid sequence having at least 90%, 91%, 92%, 93%, 94%, 95%, 96%, 97%, 98%, or 99% identity to the amino acid sequence of SEQ ID NO: 19.

In some embodiments, the bispecific antibody comprises a CLDN6 binding domain comprising a HC and a LC and a CD3 binding domain comprising an scFv and constant domain, wherein (i) the HC comprises an amino acid sequence having at least 95% identity to the amino acid sequence of SEQ ID NO: 17, (ii) the LC comprises an amino acid sequence having at least 95% identity to the amino acid sequence of SEQ ID NO: 18, and (iii) the scFv and constant domain comprises an amino acid sequence having at least 95% identity to the amino acid sequence of SEQ ID NO: 19. In some embodiments, the bispecific antibody comprises a CLDN6 binding domain comprising a HC and a LC and a CD3 binding domain comprising an scFv and constant domain, wherein (i) the HC comprises an amino acid sequence having at least 98% identity to the amino acid sequence of SEQ ID NO: 17, (ii) the LC comprises an amino acid sequence having at least 98% identity to the amino acid sequence of SEQ ID NO: 18, and (iii) the scFv and constant domain comprises an amino acid sequence having at least 98% identity to the amino acid sequence of SEQ ID NO: 19. In some embodiments, the bispecific antibody comprises a CLDN6 binding domain comprising a HC and a LC and a CD3 binding domain comprising an scFv and constant domain, wherein (i) the HC comprises the amino acid sequence of SEQ ID NO: 17, (ii) the LC comprises the amino acid sequence of SEQ ID NO: 18, and (iii) the scFv and constant domain comprises the amino acid sequence of SEQ ID NO: 19.

In some non-limiting embodiments, the bispecific antibody comprising a CLDN6 binding domain and a CD3 binding domain comprises one or more sequences as provided in Table 12 below:

TABLE 12
Exemplary bispecific antibody sequences

| Binding | | | |
|---|---|---|---|
| Domain | Region | SEQ ID NO | Sequence |
| CLDN6 | HCDR1 | SEQ ID NO: 1 | SYAMN |
| binding | | | |
| domain | | | |
| | HCDR2 | SEQ ID NO: 2 | GISSSGRYTGYADSVKG |
| | HCDR3 | SEQ ID NO: 3 | SVGSGVSWSGYVATSLDA |
| | LCDR1 | SEQ ID NO: 4 | SAGSGLYG |
| | LCDR2 | SEQ ID NO: 5 | GTNKRPS |
| | LCDR3 | SEQ ID NO: 6 | GSADSSTNAGI |
| | VH | SEQ ID NO: 7 | EVQLLESGGGLVQPGGSLRLSCAASGFTFSSYAMNWVR |
| | | | QAPGKGLEWVAGISSSGRYTGYADSVKGRFTISRDNSK |
| | | | NTLYLQMNSLRAEDTAVYYCAKSVGSGVSWSGYVATSL |
| | | | DAWGQGTLVTVSS |
| | VL | SEQ ID NO: 8 | SYELTQPPSVSVSPGQTARITCSAGSGLYGWYQQKPGQ |
| | | | APVLVIYGTNKRPSGIPERFSGSSSGTTVTLTISGVQA |
| | | | EDEADYYCGSADSSTNAGIFGGGTKLTVL |
| | HC | SEQ ID NO: 17 | EVQLLESGGGLVQPGGSLRLSCAASGFTFSSYAMNWVR |
| | | | QAPGKGLEWVAGISSSGRYTGYADSVKGRFTISRDNSK |
| | | | NTLYLQMNSLRAEDTAVYYCAKSVGSGVSWSGYVATSL |
| | | | DAWGQGTLVTVSSASTKGPSVFPLAPSSKSTSGGTAAL |
| | | | GCLVKDYFPEPVTVSWNSGALTSGVHTFPAVLQSSGLY |
| | | | SLSSVVTVPSSSLGTQTYICNVNHKPSNTKVDKKVEPK |
| | | | SCDKTHTCPPCPAPEAAGGPSVFLFPPKPKDTLMISRT |
| | | | PEVTCVVVDVSHEDPEVKFNWYVDGVEVHNAKTKPREE |
| | | | QYNSTYRVVSVLTVLHQDWLNGKEYKCKVSNKALPAPI |
| | | | EKTISKAKGQPREPQVYTLPPSREEMTKNQVSLWCLVK |
| | | | GFYPSDIAVEWESNGQPENNYKTTPPVLDSDGSFFLYS |
| | | | KLTVDKSRWQQGNVFSCSVMHEALHNHYTQKSLSLSPG |
| | | | K |
| | LC | SEQ ID NO: 18 | SYELTQPPSVSVSPGQTARITCSAGSGLYGWYQQKPGQ |
| | | | APVLVIYGTNKRPSGIPERFSGSSSGTTVTLTISGVQA |
| | | | EDEADYYCGSADSSTNAGIFGGGTKLTVLGQPKAAPSV |
| | | | TLFPPSSEELQANKATLVCLISDFYPGAVTVAWKADSS |
| | | | PVKAGVETTTPSKQSNNKYAASSYLSLTPEQWKSHRSY |
| | | | SCQVTHEGSTVEKTVAPTECS |
| CD3 | HCDR1 | SEQ ID NO: 9 | TYAMN |
| binding | | | |
| domain | | | |
| | HCDR2 | SEQ ID NO: 10 | RIRSKYNNYATYYADSVKG |
| | HCDR3 | SEQ ID NO: 11 | HGNFGDSYVSWFAY |
| | LCDR1 | SEQ ID NO: 12 | GSSTGAVTTSNYAN |
| | LCDR2 | SEQ ID NO: 13 | GTNKRAP |
| | LCDR3 | SEQ ID NO: 14 | ALWYSNHWV |
| | VH | SEQ ID NO: 15 | EVQLVESGGGLVQPGGSLRLSCAASGFTFSTYAMNWVR |
| | | | QAPGKGLEWVGRIRSKYNNYATYYADSVKGRFTISRDD |
| | | | SKNTLYLQMNSLRAEDTAVYYCVRHGNFGDSYVSWFAY |
| | | | WGQGTLVTVSS |
| | VL | SEQ ID NO: 16 | QAVVTQEPSLTVSPGGTVTLTCGSSTGAVTTSNYANWV |
| | | | QQKPGKSPRGLIGGTNKRAPGVPARFSGSLLGGKAALT |
| | | | ISGAQPEDEADYYCALWYSNHWVFGGGTKLTVL |
| | scFv | SEQ ID NO: 32 | EVQLVESGGGLVQPGGSLRLSCAASGFTFSTYAMNWVR |
| | | | QAPGKGLEWVGRIRSKYNNYATYYADSVKGRFTISRDD |
| | | | SKNTLYLQMNSLRAEDTAVYYCVRHGNFGDSYVSWFAY |
| | | | WGQGTLVTVSSGKPGSGKPGSGKPGSGKPGSQAVVTQE |
| | | | PSLTVSPGGTVTLTCGSSTGAVTTSNYANWVQQKPGKS |
| | | | PRGLIGGTNKRAPGVPARFSGSLLGGKAALTISGAQPE |
| | | | DEADYYCALWYSNHWVFGGGTKLTVL |
| | Constant | SEQ ID NO: 33 | ASPKSSDKTHTCPPCPAPEAAGGPSVFLFPPKPKDTLM |
| | domain | | ISRTPEVTCVVVDVSHEDPEVKFNWYVDGVEVHNAKTK |
| | | | PREEQYNSTYRVVSVLTVLHQDWLNGKEYKCKVSNKAL |
| | | | PAPIEKTISKAKGQPREPQVYTLPPSREEMTKNQVSLS |
| | | | CAVKGFYPSDIAVEWESNGQPENNYKTTPPVLDSDGSF |
| | | | FLVSKLTVDKSRWQQGNVFSCSVMHEALHNHYTQKSLS |
| | | | LSPGK |
| | scFv + | SEQ ID NO: 19 | EVQLVESGGGLVQPGGSLRLSCAASGFTFSTYAMNWVR |
| | constant | | QAPGKGLEWVGRIRSKYNNYATYYADSVKGRFTISRDD |
| | domain | | SKNTLYLQMNSLRAEDTAVYYCVRHGNFGDSYVSWFAY |
| | | | WGQGTLVTVSSGKPGSGKPGSGKPGSGKPGSQAVVTQE |
| | | | PSLTVSPGGTVTLTCGSSTGAVTTSNYANWVQQKPGKS |
| | | | PRGLIGGTNKRAPGVPARFSGSLLGGKAALTISGAQPE |
| | | | DEADYYCALWYSNHWVFGGGTKLTVLASPKSSDKTHTC |
| | | | PPCPAPEAAGGPSVFLFPPKPKDTLMISRTPEVTCVVV |
| | | | DVSHEDPEVKFNWYVDGVEVHNAKTKPREEQYNSTYRV |
| | | | VSVLTVLHQDWLNGKEYKCKVSNKALPAPIEKTISKAK |
| | | | GQPREPQVYTLPPSREEMTKNQVSLSCAVKGFYPSDIA |
| | | | VEWESNGQPENNYKTTPPVLDSDGSFFLVSKLTVDKSR |
| | | | WQQGNVFSCSVMHEALHNHYTQKSLSLSPGK |

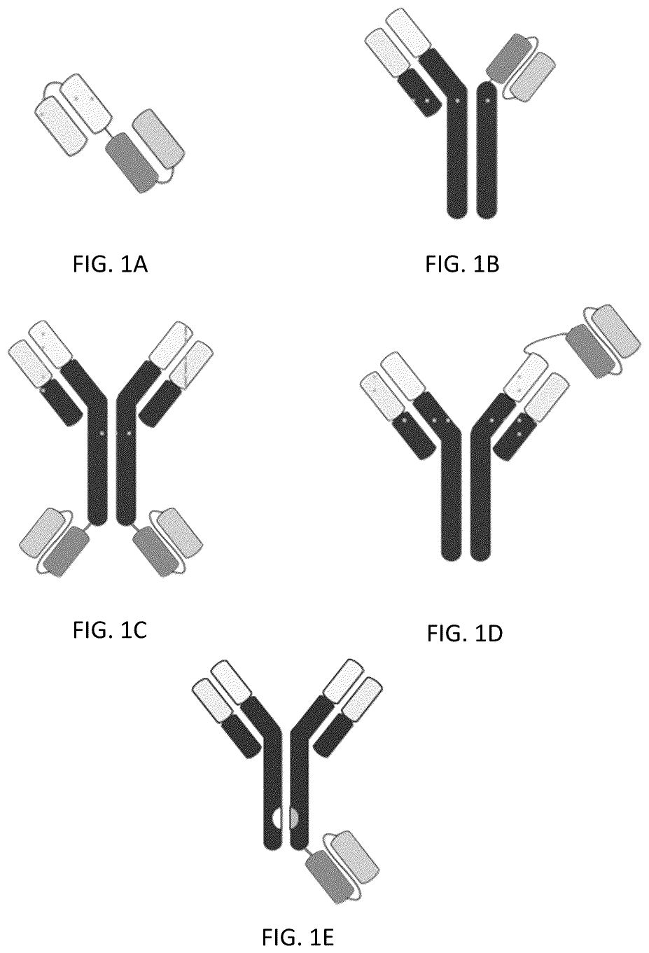

As provided for herein, the bispecific antibody comprising a CLDN6 binding domain and a CD3 binding domain is provided in various formats. Exemplary bispecific antibody formats are provided in FIG. 1.

In some embodiments, the bispecific antibody comprising a CLDN6 binding domain and a CD3 binding domain is a tandem scFv bispecific antibody, wherein the CLDN6 binding domain comprises a first V_{H} and a first V_{L} and the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, (iii) the second V_{H} comprises an amino acid sequence having at least 90%, 91%, 92%, 93%, 94%, 95%, 96%, 97%, 98%, or 99% identity to the amino acid sequence of SEQ ID NO: 15 as provided for herein, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16 as provided for herein.

In some embodiments, the bispecific antibody comprising a CLDN6 binding domain and a CD3 binding domain is a tandem scFv bispecific antibody, wherein the CLDN6 binding domain comprises a first V_{H} and a first V_{L} and the CD3 binding domain comprises a scFv, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, and (iii) the scFv comprises an amino acid sequence having at least 90%, 91%, 92%, 93%, 94%, 95%, 96%, 97%, 98%, or 99% identity to the amino acid sequence of SEQ ID NO: 32 as provided for herein.

In some embodiments, the bispecific antibody comprising a CLDN6 binding domain and a CD3 binding domain is an IgG-(scFv)₂ bispecific antibody, wherein the CLDN6 binding domain comprises a first V_{H} and a first V_{L} and the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, (iii) the second V_{H} comprises an amino acid sequence having at least 90%, 91%, 92%, 93%, 94%, 95%, 96%, 97%, 98%, or 99% identity to the amino acid sequence of SEQ ID NO: 15 as provided for herein, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16 as provided for herein.

In some embodiments, the bispecific antibody comprising a CLDN6 binding domain and a CD3 binding domain is an IgG-(scFv)₂ bispecific antibody, wherein the CLDN6 binding domain comprises a HC and a LC and the CD3 binding domain comprises a scFv, wherein (i) the HC comprises an amino acid sequence having at least 90%, 91%, 92%, 93%, 94%, 95%, 96%, 97%, 98%, or 99% identity to the amino acid sequence of SEQ ID NO: 17 as provided for herein, (ii) the LC comprises an amino acid sequence having at least 90%, 91%, 92%, 93%, 94%, 95%, 96%, 97%, 98%, or 99% identity to the amino acid sequence of SEQ ID NO: 18 as provided for herein, and (iii) the scFv comprises an amino acid sequence having at least 90%, 91%, 92%, 93%, 94%, 95%, 96%, 97%, 98%, or 99% identity to the amino acid sequence of SEQ ID NO: 32 as provided for herein.

In some embodiments, the bispecific antibody comprising a CLDN6 binding domain and a CD3 binding domain is an IgG-scFv bispecific antibody, wherein the CLDN6 binding domain comprises a first V_{H} and a first V_{L} and the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, (iii) the second V_{H} comprises an amino acid sequence having at least 90%, 91%, 92%, 93%, 94%, 95%, 96%, 97%, 98%, or 99% identity to the amino acid sequence of SEQ ID NO: 15 as provided for herein, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16 as provided for herein.

In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein (i) the HC comprises an amino acid sequence having at least 90%, 91%, 92%, 93%, 94%, 95%, 96%, 97%, 98%, or 99% identity to the amino acid sequence of SEQ ID NO: 17 as provided for herein, (ii) the LC comprises an amino acid sequence having at least 90%, 91%, 92%, 93%, 94%, 95%, 96%, 97%, 98%, or 99% identity to the amino acid sequence of SEQ ID NO: 18 as provided for herein, and (iii) the scFv comprises an amino acid sequence having at least 90%, 91%, 92%, 93%, 94%, 95%, 96%, 97%, 98%, or 99% identity to the amino acid sequence of SEQ ID NO: 32 as provided for herein. In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein the scFv is linked (e.g. through a polypeptide linker) to the constant domain of the HC of the CLDN6 binding domain. In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein the scFv is linked (e.g. through a polypeptide linker) to V_{H} domain of the HC of the CLDN6 binding domain.

In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein the scFv is linked (e.g. through a polypeptide linker) to the constant domain of the first HC of the CLDN6 binding domain. In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein the scFv is linked (e.g. through a polypeptide linker) to the constant domain of the second HC of the CLDN6 binding domain. In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein the scFv is linked (e.g. through a polypeptide linker) to V_{H} domain of the first HC of the CLDN6 binding domain. In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein the scFv is linked (e.g. through a polypeptide linker) to V_{H} domain of the second HC of the CLDN6 binding domain.

In some embodiments, the bispecific antibody comprising a CLDN6 binding domain and a CD3 binding domain is a scFv-Fab-Fc bispecific antibody, wherein the CLDN6 binding domain comprises a first V_{H} and a first V_{L} and the CD3 binding domain comprises a second V_{H} and a second V_{L}, wherein (i) the first V_{H} comprises a HCDR1 comprising an amino acid sequence of SEQ ID NO: 1, a HCDR2 comprising an amino acid sequence of SEQ ID NO: 2, and a HCDR3 comprising an amino acid sequence of SEQ ID NO: 3 as provided for herein, (ii) the first V_{L} comprises a LCDR1 comprising an amino acid sequence of SEQ ID NO: 4, a LCDR2 comprising an amino acid sequence of SEQ ID NO: 5, and a LCDR3 comprising an amino acid sequence of SEQ ID NO: 6 as provided for herein, (iii) the second V_{H} comprises a HCDR1 comprising an amino acid sequence of SEQ ID NO: 9, a HCDR2 comprising an amino acid sequence of SEQ ID NO: 10, and a HCDR3 comprising an amino acid sequence of SEQ ID NO: 11 as provided for herein, and (iv) the second V_{L} comprises a LCDR1 comprising an amino acid sequence of SEQ ID NO: 12, a LCDR2 comprising an amino acid sequence of SEQ ID NO: 13, and a LCDR3 comprising an amino acid sequence of SEQ ID NO: 14 as provided for herein.

In some embodiments, the bispecific antibody comprising a CLDN6 binding domain and a CD3 binding domain is a scFv-Fab-Fc bispecific antibody, wherein the CLDN6 binding domain comprises a first V_{H} and a first V_{L} and the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, (iii) the second V_{H} comprises an amino acid sequence having at least 90%, 91%, 92%, 93%, 94%, 95%, 96%, 97%, 98%, or 99% identity to the amino acid sequence of SEQ ID NO: 15 as provided for herein, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16 as provided for herein.

In some embodiments, the bispecific antibody comprising a CLDN6 binding domain and a CD3 binding domain is a scFv-Fab-Fc bispecific antibody, wherein the CLDN6 binding domain comprises a first V_{H} and a first V_{L} and the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7, provided that the first V_{H} comprises a HCDR1 comprising an amino acid sequence of SEQ ID NO: 1, a HCDR2 comprising an amino acid sequence of SEQ ID NO: 2, and a HCDR3 comprising an amino acid sequence of SEQ ID NO: 3 as provided for herein, (ii) the first VL comprises an amino acid sequence having at least 90%, 91%, 92%, 93%, 94%, 95%, 96%, 97%, 98%, or 99% identity to the amino acid sequence of SEQ ID NO: 8, provided that the first V_{L} comprises a LCDR1 comprising an amino acid sequence of SEQ ID NO: 4, a LCDR2 comprising an amino acid sequence of SEQ ID NO: 5, and a LCDR3 comprising an amino acid sequence of SEQ ID NO: 6 as provided for herein, (iii) the second V_{H} comprises an amino acid sequence having at least 90%, 91%, 92%, 93%, 94%, 95%, 96%, 97%, 98%, or 99% identity to the amino acid sequence of SEQ ID NO: 15, provided that the first V_{H} comprises a HCDR1 comprising an amino acid sequence of SEQ ID NO: 9, a HCDR2 comprising an amino acid sequence of SEQ ID NO: 10, and a HCDR3 comprising an amino acid sequence of SEQ ID NO: 11 as provided for herein, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16, provided that the first V_{L} comprises a LCDR1 comprising an amino acid sequence of SEQ ID NO: 12, a LCDR2 comprising an amino acid sequence of SEQ ID NO: 13, and a LCDR3 comprising an amino acid sequence of SEQ ID NO: 14 as provided for herein.

In some embodiments, the bispecific antibody comprising a CLDN6 binding domain and a CD3 binding domain is a scFv-Fab-Fc bispecific antibody, wherein the CLDN6 binding domain comprises a first V_{H} and a first V_{L} and the CD3 binding domain comprises an scFv, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, and (iii) the scFv comprises an amino acid sequence having at least 90%, 91%, 92%, 93%, 94%, 95%, 96%, 97%, 98%, or 99% identity to the amino acid sequence of SEQ ID NO: 32 as provided for herein.

In some embodiments, the bispecific antibody comprising a CLDN6 binding domain and a CD3 binding domain is a scFv-Fab-Fc bispecific antibody, wherein the CLDN6 binding domain comprises a first V_{H} and a first V_{L} and the CD3 binding domain comprises an scFv and constant domain, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, and (iii) the scFv and constant domain comprises an amino acid sequence having at least 90%, 91%, 92%, 93%, 94%, 95%, 96%, 97%, 98%, or 99% identity to the amino acid sequence of SEQ ID NO: 19 as provided for herein.

In some embodiments, the bispecific antibody comprising a CLDN6 binding domain and a CD3 binding domain is a scFv-Fab-Fc bispecific antibody, wherein the CLDN6 binding domain comprises a HC and a first LC and the CD3 binding domain comprises an scFv, wherein (i) the HC comprises an amino acid sequence having at least 90%, 91%, 92%, 93%, 94%, 95%, 96%, 97%, 98%, or 99% identity to the amino acid sequence of SEQ ID NO: 17 as provided for herein, (ii) the LC comprises an amino acid sequence having at least 90%, 91%, 92%, 93%, 94%, 95%, 96%, 97%, 98%, or 99% identity to the amino acid sequence of SEQ ID NO: 18 as provided for herein, and (iii) the scFv comprises an amino acid sequence having at least 90%, 91%, 92%, 93%, 94%, 95%, 96%, 97%, 98%, or 99% identity to the amino acid sequence of SEQ ID NO: 32 as provided for herein.

In some embodiments, the bispecific antibody comprising a CLDN6 binding domain and a CD3 binding domain is a scFv-Fab-Fc bispecific antibody, wherein the CLDN6 binding domain comprises a HC and a LC and the CD3 binding domain comprises an scFv and constant domain, wherein (i) the HC comprises an amino acid sequence having at least 90%, 91%, 92%, 93%, 94%, 95%, 96%, 97%, 98%, or 99% identity to the amino acid sequence of SEQ ID NO: 17 as provided for herein, (ii) the LC comprises an amino acid sequence having at least 90%, 91%, 92%, 93%, 94%, 95%, 96%, 97%, 98%, or 99% identity to the amino acid sequence of SEQ ID NO: 18 as provided for herein, and (iii) the scFv and constant domain comprises an amino acid sequence having at least 90%, 91%, 92%, 93%, 94%, 95%, 96%, 97%, 98%, or 99% identity to the amino acid sequence of SEQ ID NO: 19 as provided for herein.

The foregoing embodiments describing the bispecific antibody comprising a CLDN6 binding domain and a CD3 binding domain are exemplary only and are not to be construed to be limiting in any way. Additional exemplary bispecific antibodies and bispecific antibody formats that are within the scope of the present disclosure may be found at least in U.S. Publication Nos. US 2022/0127355, US 2023/0220066A1, US 2023/0057904A1, and US 2024/0132584A1, U.S. Pat. Nos. 10,738,132B2, 10,968,276B2, 11,053,316B2, 11,248,046, and 11,739,144B2, and International Publication Serial Nos. WO 2022/192403A1, WO 2021/006328A1, WO 2021/200939A1, WO 2010/094499A1, WO 2020/168059A1, WO 2022/096700A1, WO 2023/05328A1, and WO 2023/054421A1, each of which are incorporated by reference herein in their entirety.

### Pharmaceutical Compositions

In some embodiments, a pharmaceutical composition of the present disclosure comprises a bispecific antibody. In some embodiments, the bispecific antibody is as provided for herein.

In some embodiments, the pharmaceutical composition comprises a bispecific antibody in a concentration of about 1 mg/mL to about 50 mg/mL. In some embodiments, the pharmaceutical composition comprises a bispecific antibody in a concentration of about 1 mg/mL to about 40 mg/mL. In some embodiments, the pharmaceutical composition comprises a bispecific antibody in a concentration of about 1 mg/mL to about 30 mg/mL. In some embodiments, the pharmaceutical composition comprises a bispecific antibody in a concentration of about 1 mg/mL to about 25 mg/mL. In some embodiments, the pharmaceutical composition comprises a bispecific antibody in a concentration of about 1 mg/mL to about 20 mg/mL. In some embodiments, the pharmaceutical composition comprises a bispecific antibody in a concentration of about 1 mg/mL to about 15 mg/mL. In some embodiments, the pharmaceutical composition comprises a bispecific antibody in a concentration of about 1 mg/mL to about 10 mg/mL. In some embodiments, the pharmaceutical composition comprises a bispecific antibody in a concentration of about 1 mg/mL to about 5 mg/mL.

In some embodiments, the pharmaceutical composition comprises a bispecific antibody in a concentration of about 1 mg/mL to about 50 mg/mL. In some embodiments, the pharmaceutical composition comprises a bispecific antibody in a concentration of about 5 mg/mL to about 50 mg/mL. In some embodiments, the pharmaceutical composition comprises a bispecific antibody in a concentration of about 10 mg/mL to about 50 mg/mL. In some embodiments, the pharmaceutical composition comprises a bispecific antibody in a concentration of about 15 mg/mL to about 50 mg/mL. In some embodiments, the pharmaceutical composition comprises a bispecific antibody in a concentration of about 20 mg/mL to about 50 mg/mL. In some embodiments, the pharmaceutical composition comprises a bispecific antibody in a concentration of about 25 mg/mL to about 50 mg/mL. In some embodiments, the pharmaceutical composition comprises a bispecific antibody in a concentration of about 30 mg/mL to about 50 mg/mL. In some embodiments, the pharmaceutical composition comprises a bispecific antibody in a concentration of about 40 mg/mL to about 50 mg/mL.

In some embodiments, the pharmaceutical composition comprises a bispecific antibody in a concentration of about 1 mg/mL. In some embodiments, the pharmaceutical composition comprises a bispecific antibody in a concentration of about 2 mg/mL. In some embodiments, the pharmaceutical composition comprises a bispecific antibody in a concentration of about 3 mg/mL. In some embodiments, the pharmaceutical composition comprises a bispecific antibody in a concentration of about 4 mg/mL. In some embodiments, the pharmaceutical composition comprises a bispecific antibody in a concentration of about 5 mg/mL. In some embodiments, the pharmaceutical composition comprises a bispecific antibody in a concentration of about 6 mg/mL. In some embodiments, the pharmaceutical composition comprises a bispecific antibody in a concentration of about 7 mg/mL. In some embodiments, the pharmaceutical composition comprises a bispecific antibody in a concentration of about 8 mg/mL. In some embodiments, the pharmaceutical composition comprises a bispecific antibody in a concentration of about 9 mg/mL. In some embodiments, the pharmaceutical composition comprises a bispecific antibody in a concentration of about 10 mg/mL. In some embodiments, the pharmaceutical composition comprises a bispecific antibody in a concentration of about 11 mg/mL. In some embodiments, the pharmaceutical composition comprises a bispecific antibody in a concentration of about 12 mg/mL. In some embodiments, the pharmaceutical composition comprises a bispecific antibody in a concentration of about 13 mg/mL. In some embodiments, the pharmaceutical composition comprises a bispecific antibody in a concentration of about 14 mg/mL. In some embodiments, the pharmaceutical composition comprises a bispecific antibody in a concentration of about 15 mg/mL. In some embodiments, the pharmaceutical composition comprises a bispecific antibody in a concentration of about 16 mg/mL. In some embodiments, the pharmaceutical composition comprises a bispecific antibody in a concentration of about 17 mg/mL. In some embodiments, the pharmaceutical composition comprises a bispecific antibody in a concentration of about 18 mg/mL. In some embodiments, the pharmaceutical composition comprises a bispecific antibody in a concentration of about 19 mg/mL. In some embodiments, the pharmaceutical composition comprises a bispecific antibody in a concentration of about 20 mg/mL. In some embodiments, the pharmaceutical composition comprises a bispecific antibody in a concentration of about 25 mg/mL. In some embodiments, the pharmaceutical composition comprises a bispecific antibody in a concentration of about 30 mg/mL. In some embodiments, the pharmaceutical composition comprises a bispecific antibody in a concentration of about 35 mg/mL. In some embodiments, the pharmaceutical composition comprises a bispecific antibody in a concentration of about 40 mg/mL. In some embodiments, the pharmaceutical composition comprises a bispecific antibody in a concentration of about 45 mg/mL. In some embodiments, the pharmaceutical composition comprises a bispecific antibody in a concentration of about 50 mg/mL.

### Buffer

In some embodiments, a pharmaceutical composition of the present disclosure comprises a pharmaceutically acceptable buffer.

In some embodiments, the pharmaceutically acceptable buffer has a concentration of about 5 mM to about 100 mM, about 5 mM to about 90 mM, about 5 mM to about 80 mM, about 5 mM to about 70 mM, about 5 mM to about 60 mM, about 5 mM to about 50 mM, about 5 mM to about 40 mM, about 5 mM to about 30 mM, about 5 mM to about 25 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 25 mM, about 15 mM to about 100 mM, about 15 mM to about 90 mM, about 15 mM to about 80 mM, about 15 mM to about 70 mM, about 15 mM to about 60 mM, about 15 mM to about 50 mM, about 15 mM to about 40 mM, about 15 mM to about 30 mM, or about 15 mM to about 25 mM.

In some embodiments, the pharmaceutically acceptable buffer has a concentration of about 5 mM to about 100 mM, about 5 mM to about 70 mM, about 5 mM to about 60 mM, about 10 mM to about 60 mM, about 10 mM to about 50 mM, about 10 mM to about 40 mM, about 15 mM to about 30 mM, or about 15 mM to about 25 mM.

In some embodiments, the pharmaceutically acceptable buffer has a concentration of about 5 mM. In some embodiments, the pharmaceutically acceptable buffer has a concentration of about 10 mM. In some embodiments, the pharmaceutically acceptable buffer has a concentration of about 11 mM. In some embodiments, the pharmaceutically acceptable buffer has a concentration of about 12 mM. In some embodiments, the pharmaceutically acceptable buffer has a concentration of about 13 mM. In some embodiments, the pharmaceutically acceptable buffer has a concentration of about 14 mM. In some embodiments, the pharmaceutically acceptable buffer has a concentration of about 15 mM. In some embodiments, the histidine buffer has a concentration of about 16 mM. In some embodiments, the pharmaceutically acceptable buffer has a concentration of about 17 mM. In some embodiments, the pharmaceutically acceptable buffer has a concentration of about 18 mM. In some embodiments, the pharmaceutically acceptable buffer has a concentration of about 19 mM. In some embodiments, the pharmaceutically acceptable buffer has a concentration of about 20 mM. In some embodiments, the pharmaceutically acceptable buffer has a concentration of about 25 mM. In some embodiments, the pharmaceutically acceptable buffer has a concentration of about 30 mM. In some embodiments, the pharmaceutically acceptable buffer has a concentration of about 35 mM. In some embodiments, the pharmaceutically acceptable buffer has a concentration of about 40 mM. In some embodiments, the pharmaceutically acceptable buffer has a concentration of about 45 mM. In some embodiments, the pharmaceutically acceptable buffer has a concentration of about 50 mM. In some embodiments, the pharmaceutically acceptable buffer has a concentration of about 55 mM. In some embodiments, the pharmaceutically acceptable buffer has a concentration of about 60 mM. In some embodiments, the pharmaceutically acceptable buffer has a concentration of about 65 mM. In some embodiments, the pharmaceutically acceptable buffer has a concentration of about 70 mM. In some embodiments, the pharmaceutically acceptable buffer has a concentration of about 75 mM. In some embodiments, the pharmaceutically acceptable buffer has a concentration of about 80 mM. In some embodiments, the pharmaceutically acceptable buffer has a concentration of about 85 mM. In some embodiments, the pharmaceutically acceptable buffer has a concentration of about 90 mM. In some embodiments, the pharmaceutically acceptable buffer has a concentration of about 95 mM. In some embodiments, the pharmaceutically acceptable buffer has a concentration of about 100 mM. In some embodiments, the pharmaceutically acceptable buffer has a concentration of about 20 mM. In some embodiments, the pharmaceutically acceptable buffer has a concentration of 20 mM.

Examples of buffers acceptable for use with the pharmaceutical compositions disclosed herein, include, but are not limited to, histidine buffer, HEPES, phosphoric acid buffer, citric acid buffer, acetic acid buffer, succinic acid buffer, phosphate buffer, acetate buffer, citrate buffer, succinate buffer, ascorbic acid buffer, lactic acid buffer, maleic acid buffer, trometamol buffer, gluconic acid buffer, or Tris buffer, or any combination thereof.

In some embodiments, the pharmaceutically acceptable buffer is histidine buffer. In some embodiments, the pharmaceutically acceptable buffer is HEPES. In some embodiments, the pharmaceutically acceptable buffer is phosphoric acid buffer. In some embodiments, the pharmaceutically acceptable buffer is citric acid buffer. In some embodiments, the pharmaceutically acceptable buffer is acetic acid buffer. In some embodiments, the pharmaceutically acceptable buffer is succinic acid buffer. In some embodiments, the pharmaceutically acceptable buffer is phosphate buffer. In some embodiments, the pharmaceutically acceptable buffer is acetate buffer. In some embodiments, the pharmaceutically acceptable buffer is citrate buffer. In some embodiments, the pharmaceutically acceptable buffer is succinate buffer. In some embodiments, the pharmaceutically acceptable buffer is ascorbic acid buffer. In some embodiments, the pharmaceutically acceptable buffer is lactic acid buffer. In some embodiments, the pharmaceutically acceptable buffer is maleic acid buffer. In some embodiments, the pharmaceutically acceptable buffer is trometamol buffer. In some embodiments, the pharmaceutically acceptable buffer is gluconic acid buffer. In some embodiments, the pharmaceutically acceptable buffer is Tris buffer.

In some embodiments, the pharmaceutically acceptable buffer of the present disclosure is a histidine buffer.

In some embodiments, the histidine buffer has a concentration of about 5 mM to about 100 mM, about 5 mM to about 90 mM, about 5 mM to about 80 mM, about 5 mM to about 70 mM, about 5 mM to about 60 mM, about 5 mM to about 50 mM, about 5 mM to about 40 mM, about 5 mM to about 30 mM, about 5 mM to about 25 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 25 mM, about 15 mM to about 100 mM, about 15 mM to about 90 mM, about 15 mM to about 80 mM, about 15 mM to about 70 mM, about 15 mM to about 60 mM, about 15 mM to about 50 mM, about 15 mM to about 40 mM, about 15 mM to about 30 mM, or about 15 mM to about 25 mM.

In some embodiments, the histidine buffer has a concentration of about 5 mM to about 100 mM, about 5 mM to about 70 mM, about 5 mM to about 60 mM, about 10 mM to about 60 mM, about 10 mM to about 50 mM, about 10 mM to about 40 mM, about 15 mM to about 30 mM, or about 15 mM to about 25 mM.

In some embodiments, the histidine buffer has a concentration of about 5 mM. In some embodiments, the histidine buffer has a concentration of about 10 mM. In some embodiments, the histidine buffer has a concentration of about 11 mM. In some embodiments, the histidine buffer has a concentration of about 12 mM. In some embodiments, the histidine buffer has a concentration of about 13 mM. In some embodiments, the histidine buffer has a concentration of about 14 mM. In some embodiments, the histidine buffer has a concentration of about 15 mM. In some embodiments, the histidine buffer has a concentration of about 16 mM. In some embodiments, the histidine buffer has a concentration of about 17 mM. In some embodiments, the histidine buffer has a concentration of about 18 mM. In some embodiments, the histidine buffer has a concentration of about 19 mM. In some embodiments, the histidine buffer has a concentration of about 20 mM. In some embodiments, the histidine buffer has a concentration of about 25 mM. In some embodiments, the histidine buffer has a concentration of about 30 mM. In some embodiments, the histidine buffer has a concentration of about 35 mM. In some embodiments, the histidine buffer has a concentration of about 40 mM. In some embodiments, the histidine buffer has a concentration of about 45 mM. In some embodiments, the histidine buffer has a concentration of about 50 mM. In some embodiments, histidine buffer has a concentration of about 55 mM. In some embodiments, the histidine buffer has a concentration of about 60 mM. In some embodiments, the histidine buffer has a concentration of about 65 mM. In some embodiments, the histidine buffer has a concentration of about 70 mM. In some embodiments, the histidine buffer has a concentration of about 75 mM. In some embodiments, the histidine buffer has a concentration of about 80 mM. In some embodiments, the histidine buffer has a concentration of about 85 mM. In some embodiments, the histidine buffer has a concentration of about 90 mM. In some embodiments, the histidine buffer has a concentration of about 95 mM. In some embodiments, the histidine buffer has a concentration of about 100 mM. In some embodiments, the histidine buffer has a concentration of about 20 mM. In some embodiments, the histidine buffer has a concentration of 20 mM.

In some embodiments, the pharmaceutically acceptable buffer of the present disclosure is a citric acid buffer. In some embodiments, the citric acid buffer has a concentration of about 5 mM to about 100 mM.

In some embodiments, the pharmaceutically acceptable buffer of the present disclosure is a phosphate buffer. In some embodiments, the phosphate buffer has a concentration of about 5 mM to about 100 mM.

In some embodiments, the pharmaceutically acceptable buffer of the present disclosure is a Tris buffer. In some embodiments, the Tris buffer has a concentration of about 5 mM to about 100 mM.

In some embodiments, the pharmaceutical composition has a pH of about 4 to about 8, about 5 to about 7, about 5.5 to about 7, about 6 to about 7, about 6.5 to about 7, about 5 to about 6.5, about 5 to about 6, about 5 to about 6.5, about 5.5 to about 6.5, or any value or range therein. In some embodiments, the pH is about 5.9 to about 6.1.

In some embodiments, the pharmaceutical composition has a pH of about 4.0. In some embodiments, the pharmaceutical composition has a pH of about 5.0. In some embodiments, the pharmaceutical composition has a pH of about 5.1. In some embodiments, the pharmaceutical composition has a pH of about 5.2. In some embodiments, the pharmaceutical composition has a pH of about 5.3. In some embodiments, the pharmaceutical composition has a pH of about 5.4. In some embodiments, the pharmaceutical composition has a pH of about 5.5. In some embodiments, the pharmaceutical composition has a pH of about 5.6. In some embodiments, the pharmaceutical composition has a pH of about 5.7. In some embodiments, the pharmaceutical composition has a pH of about 5.8. In some embodiments, the pharmaceutical composition has a pH of about 5.9. In some embodiments, the pharmaceutical composition has a pH of about 6.0. In some embodiments, the pharmaceutical composition has a pH of about 6.1. In some embodiments, the pharmaceutical composition has a pH of about 6.2. In some embodiments, the pharmaceutical composition has a pH of about 6.3. In some embodiments, the pharmaceutical composition has a pH of about 6.4. In some embodiments, the pharmaceutical composition has a pH of about 6.5. In some embodiments, the pharmaceutical composition has a pH of about 6.6. In some embodiments, the pharmaceutical composition has a pH of about 6.7. In some embodiments, the pharmaceutical composition has a pH of about 6.8. In some embodiments, the pharmaceutical composition has a pH of about 6.9. In some embodiments, the pharmaceutical composition has a pH of about 7.0. In some embodiments, the pharmaceutical composition has a pH of about 8.0.

In some embodiments, the pharmaceutically acceptable buffer is a histidine buffer and the pharmaceutical composition has a pH of about 5 to about 7. In some embodiments, the pharmaceutically acceptable buffer is a histidine buffer and the pharmaceutical composition has a pH of about 5.5 to about 6.5. In some embodiments, the pharmaceutically acceptable buffer is a histidine buffer and the pharmaceutical composition has a pH of about 5.9 to about 6.1.

In some embodiments, the pharmaceutically acceptable buffer is a histidine buffer and the pharmaceutical composition has a pH of about 5.0. In some embodiments, the pharmaceutically acceptable buffer is a histidine buffer and the pharmaceutical composition has a pH of about 5.1. In some embodiments, the pharmaceutically acceptable buffer is a histidine buffer and the pharmaceutical composition has a pH of about 5.2. In some embodiments, the pharmaceutically acceptable buffer is a histidine buffer and the pharmaceutical composition has a pH of about 5.3. In some embodiments, the pharmaceutically acceptable buffer is a histidine buffer and the pharmaceutical composition has a pH of about 5.4. In some embodiments, the pharmaceutically acceptable buffer is a histidine buffer and the pharmaceutical composition has a pH of about 5.5. In some embodiments, the pharmaceutically acceptable buffer is a histidine buffer and the pharmaceutical composition has a pH of about 5.6. In some embodiments, the pharmaceutically acceptable buffer is a histidine buffer and the pharmaceutical composition has a pH of about 5.7. In some embodiments, the pharmaceutically acceptable buffer is a histidine buffer and the pharmaceutical composition has a pH of about 5.8. In some embodiments, the pharmaceutically acceptable buffer is a histidine buffer and the pharmaceutical composition has a pH of about 5.9. In some embodiments, the pharmaceutically acceptable buffer is a histidine buffer and the pharmaceutical composition has a pH of about 6.0. In some embodiments, the pharmaceutically acceptable buffer is a histidine buffer and the pharmaceutical composition has a pH of about 6.0. In some embodiments, the pharmaceutically acceptable buffer is a histidine buffer and the pharmaceutical composition has a pH of about 6.1. In some embodiments, the pharmaceutically acceptable buffer is a histidine buffer and the pharmaceutical composition has a pH of about 6.2. In some embodiments, the pharmaceutically acceptable buffer is a histidine buffer and the pharmaceutical composition has a pH of about 6.3. In some embodiments, the pharmaceutically acceptable buffer is a histidine buffer and the pharmaceutical composition has a pH of about 6.4. In some embodiments, the pharmaceutically acceptable buffer is a histidine buffer and the pharmaceutical composition has a pH of about 6.5. In some embodiments, the pharmaceutically acceptable buffer is a histidine buffer and the pharmaceutical composition has a pH of about 6.6. In some embodiments, the pharmaceutically acceptable buffer is a histidine buffer and the pharmaceutical composition has a pH of about 6.7. In some embodiments, the pharmaceutically acceptable buffer is a histidine buffer and the pharmaceutical composition has a pH of about 6.8. In some embodiments, the pharmaceutically acceptable buffer is a histidine buffer and the pharmaceutical composition has a pH of about 6.9. In some embodiments, the pharmaceutically acceptable buffer is a histidine buffer and the pharmaceutical composition has a pH of about 7.0.

In some embodiments, the pharmaceutically acceptable buffer is a histidine buffer and the pharmaceutical composition has a pH of about 6.0 or about 5.9 to about 6.1.

### Salts

In some embodiments, the pharmaceutical composition does not comprise a salt, such as a pharmaceutically acceptable salt.

In some embodiments, the pharmaceutical composition comprises a pharmaceutically acceptable salt.

In some embodiments, the pharmaceutically acceptable salt is sodium chloride.

In some embodiments, the pharmaceutically acceptable salt is present at about 50 mM to 300 mM. In some embodiments, the pharmaceutically acceptable salt is present at about 50 mM to 275 mM. In some embodiments, the pharmaceutically acceptable salt is present at about 50 mM to 250 mM. In some embodiments, the pharmaceutically acceptable salt is present at about 50 mM to 225 mM. In some embodiments, the pharmaceutically acceptable salt is present at about 50 mM to 200 mM. In some embodiments, the pharmaceutically acceptable salt is present at about 50 mM to 175 mM. In some embodiments, the pharmaceutically acceptable salt is present at about 50 mM to 150 mM. In some embodiments, the pharmaceutically acceptable salt is present at about 50 mM to 125 mM. In some embodiments, the pharmaceutically acceptable salt is present at about 50 mM to 100 mM. In some embodiments, the pharmaceutically acceptable salt is present at about 50 mM to 75 mM.

In some embodiments, the pharmaceutically acceptable salt is present at about 50 mM to 300 mM. In some embodiments, the pharmaceutically acceptable salt is present at about 75 mM to 300 mM. In some embodiments, the pharmaceutically acceptable salt is present at about 100 mM to 300 mM. In some embodiments, the pharmaceutically acceptable salt is present at about 125 mM to 300 mM. In some embodiments, the pharmaceutically acceptable salt is present at about 150 mM to 300 mM. In some embodiments, the pharmaceutically acceptable salt is present at about 175 mM to 300 mM. In some embodiments, the pharmaceutically acceptable salt is present at about 200 mM to 300 mM. In some embodiments, the pharmaceutically acceptable salt is present at about 225 mM to 300 mM. In some embodiments, the pharmaceutically acceptable salt is present at about 250 mM to 300 mM. In some embodiments, the pharmaceutically acceptable salt is present at about 275 mM to 300 mM.

In some embodiments, the pharmaceutically acceptable salt is present at about 50 mM. In some embodiments, the pharmaceutically acceptable salt is present at about 75 mM. In some embodiments, the pharmaceutically acceptable salt is present at about 100 mM. In some embodiments, the pharmaceutically acceptable salt is present at about 125 mM. In some embodiments, the pharmaceutically acceptable salt is present at about 150 mM. In some embodiments, the pharmaceutically acceptable salt is present at about 175 mM. In some embodiments, the pharmaceutically acceptable salt is present at about 200 mM. In some embodiments, the pharmaceutically acceptable salt is present at about 225 mM. In some embodiments, the pharmaceutically acceptable salt is present at about 250 mM. In some embodiments, the pharmaceutically acceptable salt is present at about 275 mM. In some embodiments, the pharmaceutically acceptable salt is present at about 300 mM.

In some embodiments, the pharmaceutically acceptable salt is sodium chloride.

In some embodiments, the sodium chloride is present at about 50 mM to 300 mM. In some embodiments, the sodium chloride is present at about 50 mM to 275 mM. In some embodiments, the sodium chloride is present at about 50 mM to 250 mM. In some embodiments, the sodium chloride is present at about 50 mM to 225 mM. In some embodiments, the sodium chloride is present at about 50 mM to 200 mM. In some embodiments, the sodium chloride is present at about 50 mM to 175 mM. In some embodiments, the sodium chloride is present at about 50 mM to 150 mM. In some embodiments, the sodium chloride is present at about 50 mM to 125 mM. In some embodiments, the sodium chloride is present at about 50 mM to 100 mM. In some embodiments, the sodium chloride is present at about 50 mM to 75 mM.

In some embodiments, the sodium chloride is present at about 50 mM to 300 mM. In some embodiments, the sodium chloride is present at about 75 mM to 300 mM. In some embodiments, the sodium chloride is present at about 100 mM to 300 mM. In some embodiments, the sodium chloride is present at about 125 mM to 300 mM. In some embodiments, the sodium chloride is present at about 150 mM to 300 mM. In some embodiments, the sodium chloride is present at about 175 mM to 300 mM. In some embodiments, the sodium chloride is present at about 200 mM to 300 mM. In some embodiments, the sodium chloride is present at about 225 mM to 300 mM. In some embodiments, the sodium chloride is present at about 250 mM to 300 mM. In some embodiments, the sodium chloride is present at about 275 mM to 300 mM.

In some embodiments, the sodium chloride is present at about 50 mM. In some embodiments, the sodium chloride is present at about 75 mM. In some embodiments, the sodium chloride is present at about 100 mM. In some embodiments, the sodium chloride is present at about 125 mM. In some embodiments, the sodium chloride is present at about 150 mM. In some embodiments, the sodium chloride is present at about 175 mM. In some embodiments, the sodium chloride is present at about 200 mM. In some embodiments, the sodium chloride is present at about 225 mM. In some embodiments, the sodium chloride is present at about 250 mM. In some embodiments, the sodium chloride is present at about 275 mM. In some embodiments, the sodium chloride is present at about 300 mM.

In some embodiments, the composition does not contain or comprise sodium chloride.

### Sugars

In some embodiments, the pharmaceutical compositions disclosed herein comprise a sugar. Without wishing to be bound by any theory, sugars added as excipients to pharmaceutical compositions containing proteins, such as antibodies, may act as stabilizers or lyoprotectants. Furthermore, sugars can help prevent interactions between antibodies and solvents.

In some embodiments, the sugar is present at about 50 mM to about 500 mM. In some embodiments, the sugar is present at about 50 mM to about 450 mM. In some embodiments, the sugar is present at about 50 mM to about 400 mM. In some embodiments, the sugar is present at about 50 mM to about 350 mM. In some embodiments, the sugar is present at about 50 mM to about 300 mM. In some embodiments, the sugar is present at about 50 mM to about 250 mM. In some embodiments, the sugar is present at about 50 mM to about 200 mM. In some embodiments, the sugar is present at about 50 mM to about 150 mM. In some embodiments, the sugar is present at about 50 mM to about 100 mM.

In some embodiments, the sugar is present at about 50 mM to about 500 mM. In some embodiments, the sugar is present at about 100 mM to about 500 mM. In some embodiments, the sugar is present at about 150 mM to about 500 mM. In some embodiments, the sugar is present at about 200 mM to about 500 mM. In some embodiments, the sugar is present at about 250 mM to about 500 mM. In some embodiments, the sugar is present at about 300 mM to about 500 mM. In some embodiments, the sugar is present at about 350 mM to about 500 mM. In some embodiments, the sugar is present at about 400 mM to about 500 mM. In some embodiments, the sugar is present at about 450 mM to about 500 mM.

In some embodiments, the sugar is present at about 50 mM. In some embodiments, the sugar is present at about 100 mM. In some embodiments, the sugar is present at about 150 mM. In some embodiments, the sugar is present at about 160 mM. In some embodiments, the sugar is present at about 170 mM. In some embodiments, the sugar is present at about 180 mM. In some embodiments, the sugar is present at about 190 mM. In some embodiments, the sugar is present at about 200 mM. In some embodiments, the sugar is present at about 210 mM. In some embodiments, the sugar is present at about 220 mM. In some embodiments, the sugar is present at about 230 mM. In some embodiments, the sugar is present at about 240 mM. In some embodiments, the sugar is present at about 250 mM. In some embodiments, the sugar is present at about 260 mM. In some embodiments, the sugar is present at about 270 mM. In some embodiments, the sugar is present at about 280 mM. In some embodiments, the sugar is present at about 290 mM. In some embodiments, the sugar is present at about 300 mM. In some embodiments, the sugar is present at about 350 mM. In some embodiments, the sugar is present at about 400 mM. In some embodiments, the sugar is present at about 450 mM. In some embodiments, the sugar is present at about 500 mM.

In some embodiments, the sugar is present at about 240 mM.

In some embodiments, the sugar is selected from sucrose, trehalose, mannose, glucose, sorbitol, or mannitol, or any combination thereof. In some embodiments, the pharmaceutical composition comprises the sugar at a concentration of about 220 mM to about 280 mM (e.g., 200 to 300 mM). In some embodiments, the pharmaceutical composition comprises the sugar at a concentration of about 240 mM (e.g., between 220 and 260 mM). In some embodiments, the pharmaceutical composition comprises the sugar at a concentration of 240 mM. In some embodiments, the pharmaceutical composition disclosed herein comprises sucrose. In some embodiments, the pharmaceutical composition comprises about 240 mM sucrose. In some embodiments, the pharmaceutical composition comprises 240 mM sucrose.

In some embodiments, the sugar is sucrose. In some embodiments, the sugar is trehalose. In some embodiments, the sugar is mannose. In some embodiments, the sugar is glucose. In some embodiments, the sugar is sorbitol. In some embodiments, the sugar is mannitol.

In some embodiments, the sugar is sucrose.

In some embodiments, the composition does not comprise trehalose. In some embodiments, the composition does not comprise a significant amount of trehalose. In some embodiments, the composition contains less than 1%, 0.5%, 0.1%, 0.01% (w/v for a liquid composition or w/w for a lyophilized or solid composition) of trehalose. In some embodiments, the only sugar present in the composition is sucrose.

In some embodiments, the sucrose is present at about 50 mM to about 500 mM. In some embodiments, the sucrose is present at about 50 mM to about 450 mM. In some embodiments, the sucrose is present at about 50 mM to about 400 mM. In some embodiments, the sucrose is present at about 50 mM to about 350 mM. In some embodiments, the sucrose is present at about 50 mM to about 300 mM. In some embodiments, the sucrose is present at about 50 mM to about 250 mM. In some embodiments, the sucrose is present at about 50 mM to about 200 mM. In some embodiments, the sucrose is present at about 50 mM to about 150 mM. In some embodiments, the sucrose is present at about 50 mM to about 100 mM.

In some embodiments, the sucrose is present at about 50 mM to about 500 mM. In some embodiments, the sucrose is present at about 100 mM to about 500 mM. In some embodiments, the sucrose is present at about 150 mM to about 500 mM. In some embodiments, the sucrose is present at about 200 mM to about 500 mM. In some embodiments, the sucrose is present at about 250 mM to about 500 mM. In some embodiments, the sucrose is present at about 300 mM to about 500 mM. In some embodiments, the sucrose is present at about 350 mM to about 500 mM. In some embodiments, the sucrose is present at about 400 mM to about 500 mM. In some embodiments, the sucrose is present at about 450 mM to about 500 mM.

In some embodiments, the sucrose is present at about 50 mM. In some embodiments, the sucrose is present at about 100 mM. In some embodiments, the sucrose is present at about 150 mM. In some embodiments, the sucrose is present at about 160 mM. In some embodiments, the sucrose is present at about 170 mM. In some embodiments, the sucrose is present at about 180 mM. In some embodiments, the sucrose is present at about 190 mM. In some embodiments, the sucrose is present at about 200 mM. In some embodiments, the sucrose is present at about 210 mM. In some embodiments, the sucrose is present at about 220 mM. In some embodiments, the sucrose is present at about 230 mM. In some embodiments, the sucrose is present at about 240 mM. In some embodiments, the sucrose is present at about 250 mM. In some embodiments, the sucrose is present at about 260 mM. In some embodiments, the sucrose is present at about 270 mM. In some embodiments, the sucrose is present at about 280 mM. In some embodiments, the sucrose is present at about 290 mM. In some embodiments, the sucrose is present at about 300 mM. In some embodiments, the sucrose is present at about 350 mM. In some embodiments, the sucrose is present at about 400 mM. In some embodiments, the sucrose is present at about 450 mM. In some embodiments, the sucrose is present at about 500 mM.

In some embodiments, the sucrose is present at about 240 mM.

In some embodiments, the sugar is glucose. In some embodiments, the glucose is present at about 50 mM to about 500 mM.

In some embodiments, the sugar is sorbitol. In some embodiments, the sorbitol is present at about 50 mM to about 500 mM.

In some embodiments, the sugar is mannitol. In some embodiments, the mannitol is present at about 50 mM to about 500 mM.

### Non-Ionic Surfactants

In some embodiments, the pharmaceutical composition according to the present disclosure comprises a non-ionic surfactant. Surfactants are generally chemical agents that reduce the surface tension or interfacial tension between two liquids, liquid and a gas, or liquid and a solid. Due to the amphiphilic nature of proteins, including antibodies, they may be subjected to substantial interfacial stress, e.g., stress resulting from air/water interfaces during mixing of liquids, ice/water interfaces during freezing/thawing, oil/water interfaces of transmembrane proteins, and the like. Without wishing to be bound by any theory, non-ionic surfactants added as excipients to pharmaceutical compositions containing proteins, such as antibodies, may act to increase protein stability by minimizing adsorption to surfaces and interface-induced aggregation.

In some embodiments, the non-ionic surfactant concentration in the pharmaceutical composition is about 0.003% to 0.3% (w/v). In some embodiments, the non-ionic surfactant concentration in the pharmaceutical composition is about 0.003% to 0.2% (w/v). In some embodiments, the non-ionic surfactant concentration in the pharmaceutical composition is about 0.003% to 0.1% (w/v). In some embodiments, the non-ionic surfactant concentration in the pharmaceutical composition is about 0.003% to 0.09% (w/v). In some embodiments, the non-ionic surfactant concentration in the pharmaceutical composition is about 0.003% to 0.08% (w/v). In some embodiments, the non-ionic surfactant concentration in the pharmaceutical composition is about 0.003% to 0.075% (w/v). In some embodiments, the non-ionic surfactant concentration in the pharmaceutical composition is about 0.003% to 0.07% (w/v). In some embodiments, the non-ionic surfactant concentration in the pharmaceutical composition is about 0.003% to 0.065% (w/v). In some embodiments, the non-ionic surfactant concentration in the pharmaceutical composition is about 0.003% to 0.06% (w/v). In some embodiments, the non-ionic surfactant concentration in the pharmaceutical composition is about 0.003% to 0.055% (w/v). In some embodiments, the non-ionic surfactant concentration in the pharmaceutical composition is about 0.003% to 0.05% (w/v). In some embodiments, the non-ionic surfactant concentration in the pharmaceutical composition is about 0.003% to 0.045% (w/v). In some embodiments, the non-ionic surfactant concentration in the pharmaceutical composition is about 0.003% to 0.04% (w/v). In some embodiments, the non-ionic surfactant concentration in the pharmaceutical composition is about 0.003% to 0.035% (w/v). In some embodiments, the non-ionic surfactant concentration in the pharmaceutical composition is about 0.003% to 0.03% (w/v). In some embodiments, the non-ionic surfactant concentration in the pharmaceutical composition is about 0.003% to 0.025% (w/v). In some embodiments, the non-ionic surfactant concentration in the pharmaceutical composition is about 0.003% to 0.02% (w/v). In some embodiments, the non-ionic surfactant concentration in the pharmaceutical composition is about 0.003% to 0.015% (w/v). In some embodiments, the non-ionic surfactant concentration in the pharmaceutical composition is about 0.003% to 0.01% (w/v). In some embodiments, the non-ionic surfactant concentration in the pharmaceutical composition is about 0.003% to 0.009% (w/v). In some embodiments, the non-ionic surfactant concentration in the pharmaceutical composition is about 0.003% to 0.008% (w/v). In some embodiments, the non-ionic surfactant concentration in the pharmaceutical composition is about 0.003% to 0.007% (w/v). In some embodiments, the non-ionic surfactant concentration in the pharmaceutical composition is about 0.003% to 0.006% (w/v). In some embodiments, the non-ionic surfactant concentration in the pharmaceutical composition is about 0.003% to 0.005% (w/v). In some embodiments, the non-ionic surfactant concentration in the pharmaceutical composition is about 0.003% to 0.004% (w/v).

In some embodiments, the non-ionic surfactant concentration in the pharmaceutical composition is about 0.003% to 0.3% (w/v). In some embodiments, the non-ionic surfactant concentration in the pharmaceutical composition is about 0.004% to 0.3% (w/v). In some embodiments, the non-ionic surfactant concentration in the pharmaceutical composition is about 0.005% to 0.3% (w/v). In some embodiments, the non-ionic surfactant concentration in the pharmaceutical composition is about 0.006% to 0.3% (w/v). In some embodiments, the non-ionic surfactant concentration in the pharmaceutical composition is about 0.007% to 0.3% (w/v). In some embodiments, the non-ionic surfactant concentration in the pharmaceutical composition is about 0.008% to 0.3% (w/v). In some embodiments, the non-ionic surfactant concentration in the pharmaceutical composition is about 0.009% to 0.3% (w/v). In some embodiments, the non-ionic surfactant concentration in the pharmaceutical composition is about 0.01% to 0.3% (w/v). In some embodiments, the non-ionic surfactant concentration in the pharmaceutical composition is about 0.015% to 0.3% (w/v). In some embodiments, the non-ionic surfactant concentration in the pharmaceutical composition is about 0.02% to 0.3% (w/v). In some embodiments, the non-ionic surfactant concentration in the pharmaceutical composition is about 0.025% to 0.3% (w/v). In some embodiments, the non-ionic surfactant concentration in the pharmaceutical composition is about 0.03% to 0.3% (w/v). In some embodiments, the non-ionic surfactant concentration in the pharmaceutical composition is about 0.035% to 0.3% (w/v). In some embodiments, the non-ionic surfactant concentration in the pharmaceutical composition is about 0.04% to 0.3% (w/v). In some embodiments, the non-ionic surfactant concentration in the pharmaceutical composition is about 0.045% to 0.3% (w/v). In some embodiments, the non-ionic surfactant concentration in the pharmaceutical composition is about 0.05% to 0.3% (w/v). In some embodiments, the non-ionic surfactant concentration in the pharmaceutical composition is about 0.055% to 0.3% (w/v). In some embodiments, the non-ionic surfactant concentration in the pharmaceutical composition is about 0.06% to 0.3% (w/v). In some embodiments, the non-ionic surfactant concentration in the pharmaceutical composition is about 0.065% to 0.3% (w/v). In some embodiments, the non-ionic surfactant concentration in the pharmaceutical composition is about 0.07% to 0.3% (w/v). In some embodiments, the non-ionic surfactant concentration in the pharmaceutical composition is about 0.075% to 0.3% (w/v). In some embodiments, the non-ionic surfactant concentration in the pharmaceutical composition is about 0.08% to 0.3% (w/v). In some embodiments, the non-ionic surfactant concentration in the pharmaceutical composition is about 0.09% to 0.3% (w/v). In some embodiments, the non-ionic surfactant concentration in the pharmaceutical composition is about 0.1% to 0.3% (w/v). In some embodiments, the non-ionic surfactant concentration in the pharmaceutical composition is about 0.2% to 0.3% (w/v).

In some embodiments, the non-ionic surfactant concentration in the pharmaceutical composition is about 0.003% (w/v). In some embodiments, the non-ionic surfactant concentration in the pharmaceutical composition is about 0.004% (w/v). In some embodiments, the non-ionic surfactant concentration in the pharmaceutical composition is about 0.005% (w/v). In some embodiments, the non-ionic surfactant concentration in the pharmaceutical composition is about 0.006% (w/v). In some embodiments, the non-ionic surfactant concentration in the pharmaceutical composition is about 0.007% (w/v). In some embodiments, the non-ionic surfactant concentration in the pharmaceutical composition is about 0.008% (w/v). In some embodiments, the non-ionic surfactant concentration in the pharmaceutical composition is about 0.009% (w/v). In some embodiments, the non-ionic surfactant concentration in the pharmaceutical composition is about 0.01% (w/v). In some embodiments, the non-ionic surfactant concentration in the pharmaceutical composition is about 0.015% (w/v). In some embodiments, the non-ionic surfactant concentration in the pharmaceutical composition is about 0.02% (w/v). In some embodiments, the non-ionic surfactant concentration in the pharmaceutical composition is about 0.025% (w/v). In some embodiments, the non-ionic surfactant concentration in the pharmaceutical composition is about 0.03% (w/v). the non-ionic surfactant concentration in the pharmaceutical composition is about 0.035% (w/v). In some embodiments, the non-ionic surfactant concentration in the pharmaceutical composition is about 0.04% (w/v). In some embodiments, the non-ionic surfactant concentration in the pharmaceutical composition is about 0.045% (w/v). In some embodiments, the non-ionic surfactant concentration in the pharmaceutical composition is about 0.05% (w/v). In some embodiments, the non-ionic surfactant concentration in the pharmaceutical composition is about 0.055% (w/v). In some embodiments, the non-ionic surfactant concentration in the pharmaceutical composition is about 0.06% (w/v). In some embodiments, the non-ionic surfactant concentration in the pharmaceutical composition is about 0.065% (w/v). In some embodiments, the non-ionic surfactant concentration in the pharmaceutical composition is about 0.07% (w/v). In some embodiments, the non-ionic surfactant concentration in the pharmaceutical composition is about 0.075% (w/v). In some embodiments, the non-ionic surfactant concentration in the pharmaceutical composition is about 0.08% (w/v). In some embodiments, the non-ionic surfactant concentration in the pharmaceutical composition is about 0.09% (w/v). In some embodiments, the non-ionic surfactant concentration in the pharmaceutical composition is about 0.1% (w/v). In some embodiments, the non-ionic surfactant concentration in the pharmaceutical composition is about 0.2% (w/v). In some embodiments, the non-ionic surfactant concentration in the pharmaceutical composition is about 0.3% (w/v).

In some embodiments, the non-ionic surfactant concentration in the pharmaceutical composition is about 0.03% (w/v).

Examples of non-ionic surfactants suitable for use with the pharmaceutical compositions disclosed herein, include, but are not limited to, polyoxyethylensorbitan fatty acid esters (e.g., polysorbate 20 and polysorbate 80), polyethylene-polypropylene copolymers, polyethylene-polypropylene glycols, polyox ethylene-stearates, polyoxyethylene alkyl ethers, e.g., polyoxyethylene monolauryl ether, alkylphenylpolyoxyethylene ethers (Triton-X), polyoxyethylene-polyoxypropylene copolymer (Poloxamer, Pluronic), sodium dodecyl sulphate (SDS).

In some embodiments, the non-ionic surfactant is polysorbate 20, polysorbate 80, poloxamer, poloxamer 188, or poloxamer 407. In some embodiments, the non-ionic surfactant is polysorbate 20. In certain embodiments, the non-ionic surfactant is polysorbate 80. In some embodiments, the non-ionic surfactant is poloxamer. In some embodiments, the non-ionic surfactant is poloxamer 188. In some embodiments, the non-ionic surfactant is poloxamer 407.

In some embodiments, the non-ionic surfactant is polysorbate 20.

In some embodiments, the polysorbate 20 concentration in the pharmaceutical composition is about 0.003% to 0.3% (w/v). In some embodiments, the polysorbate 20 concentration in the pharmaceutical composition is about 0.003% to 0.2% (w/v). In some embodiments, the polysorbate 20 concentration in the pharmaceutical composition is about 0.003% to 0.1% (w/v). In some embodiments, the polysorbate 20 concentration in the pharmaceutical composition is about 0.003% to 0.09% (w/v). In some embodiments, the polysorbate 20 concentration in the pharmaceutical composition is about 0.003% to 0.08% (w/v). In some embodiments, the polysorbate 20 concentration in the pharmaceutical composition is about 0.003% to 0.075% (w/v). In some embodiments, the polysorbate 20 concentration in the pharmaceutical composition is about 0.003% to 0.07% (w/v). In some embodiments, the polysorbate 20 concentration in the pharmaceutical composition is about 0.003% to 0.065% (w/v). In some embodiments, the polysorbate 20 concentration in the pharmaceutical composition is about 0.003% to 0.06% (w/v). In some embodiments, the polysorbate 20 concentration in the pharmaceutical composition is about 0.003% to 0.055% (w/v). In some embodiments, the polysorbate 20 concentration in the pharmaceutical composition is about 0.003% to 0.05% (w/v). In some embodiments, the polysorbate 20 concentration in the pharmaceutical composition is about 0.003% to 0.045% (w/v). In some embodiments, the polysorbate 20 concentration in the pharmaceutical composition is about 0.003% to 0.04% (w/v). In some embodiments, the polysorbate 20 concentration in the pharmaceutical composition is about 0.003% to 0.035% (w/v). In some embodiments, the polysorbate 20 concentration in the pharmaceutical composition is about 0.003% to 0.03% (w/v). In some embodiments, the polysorbate 20 concentration in the pharmaceutical composition is about 0.003% to 0.025% (w/v). In some embodiments, the polysorbate 20 concentration in the pharmaceutical composition is about 0.003% to 0.02% (w/v). In some embodiments, the polysorbate 20 concentration in the pharmaceutical composition is about 0.003% to 0.015% (w/v). In some embodiments, the polysorbate 20 concentration in the pharmaceutical composition is about 0.003% to 0.01% (w/v). In some embodiments, the polysorbate 20 concentration in the pharmaceutical composition is about 0.003% to 0.009% (w/v). In some embodiments, the polysorbate 20 concentration in the pharmaceutical composition is about 0.003% to 0.008% (w/v). In some embodiments, the polysorbate 20 concentration in the pharmaceutical composition is about 0.003% to 0.007% (w/v). In some embodiments, the polysorbate 20 concentration in the pharmaceutical composition is about 0.003% to 0.006% (w/v). In some embodiments, the polysorbate 20 concentration in the pharmaceutical composition is about 0.003% to 0.005% (w/v). In some embodiments, the polysorbate 20 concentration in the pharmaceutical composition is about 0.003% to 0.004% (w/v).

In some embodiments, the polysorbate 20 concentration in the pharmaceutical composition is about 0.003% to 0.3% (w/v). In some embodiments, the polysorbate 20 concentration in the pharmaceutical composition is about 0.004% to 0.3% (w/v). In some embodiments, the polysorbate 20 concentration in the pharmaceutical composition is about 0.005% to 0.3% (w/v). In some embodiments, the polysorbate 20 concentration in the pharmaceutical composition is about 0.006% to 0.3% (w/v). In some embodiments, the polysorbate 20 concentration in the pharmaceutical composition is about 0.007% to 0.3% (w/v). In some embodiments, the polysorbate 20 concentration in the pharmaceutical composition is about 0.008% to 0.3% (w/v). In some embodiments, the polysorbate 20 concentration in the pharmaceutical composition is about 0.009% to 0.3% (w/v). In some embodiments, the polysorbate 20 concentration in the pharmaceutical composition is about 0.01% to 0.3% (w/v). In some embodiments, the polysorbate 20 concentration in the pharmaceutical composition is about 0.015% to 0.3% (w/v). In some embodiments, the polysorbate 20 concentration in the pharmaceutical composition is about 0.02% to 0.3% (w/v). In some embodiments, the polysorbate 20 concentration in the pharmaceutical composition is about 0.025% to 0.3% (w/v). In some embodiments, the polysorbate 20 concentration in the pharmaceutical composition is about 0.03% to 0.3% (w/v). In some embodiments, the polysorbate 20 concentration in the pharmaceutical composition is about 0.035% to 0.3% (w/v). In some embodiments, the polysorbate 20 concentration in the pharmaceutical composition is about 0.04% to 0.3% (w/v). In some embodiments, the polysorbate 20 concentration in the pharmaceutical composition is about 0.045% to 0.3% (w/v). In some embodiments, the polysorbate 20 concentration in the pharmaceutical composition is about 0.05% to 0.3% (w/v). In some embodiments, the polysorbate 20 concentration in the pharmaceutical composition is about 0.055% to 0.3% (w/v). In some embodiments, the polysorbate 20 concentration in the pharmaceutical composition is about 0.06% to 0.3% (w/v). In some embodiments, the polysorbate 20 concentration in the pharmaceutical composition is about 0.065% to 0.3% (w/v). In some embodiments, the polysorbate 20 concentration in the pharmaceutical composition is about 0.07% to 0.3% (w/v). In some embodiments, the polysorbate 20 concentration in the pharmaceutical composition is about 0.075% to 0.3% (w/v). In some embodiments, the polysorbate 20 concentration in the pharmaceutical composition is about 0.08% to 0.3% (w/v). In some embodiments, the polysorbate 20 concentration in the pharmaceutical composition is about 0.09% to 0.3% (w/v). In some embodiments, the polysorbate 20 concentration in the pharmaceutical composition is about 0.1% to 0.3% (w/v). In some embodiments, the polysorbate 20 concentration in the pharmaceutical composition is about 0.2% to 0.3% (w/v).

In some embodiments, the polysorbate 20 concentration in the pharmaceutical composition is about 0.003% (w/v). In some embodiments, the polysorbate 20 concentration in the pharmaceutical composition is about 0.004% (w/v). In some embodiments, the polysorbate 20 concentration in the pharmaceutical composition is about 0.005% (w/v). In some embodiments, the polysorbate 20 concentration in the pharmaceutical composition is about 0.006% (w/v). In some embodiments, the polysorbate 20 concentration in the pharmaceutical composition is about 0.007% (w/v). In some embodiments, the polysorbate 20 concentration in the pharmaceutical composition is about 0.008% (w/v). In some embodiments, the polysorbate 20 concentration in the pharmaceutical composition is about 0.009% (w/v). In some embodiments, the polysorbate 20 concentration in the pharmaceutical composition is about 0.01% (w/v). In some embodiments, the polysorbate 20 concentration in the pharmaceutical composition is about 0.015% (w/v). In some embodiments, the polysorbate 20 concentration in the pharmaceutical composition is about 0.02% (w/v). In some embodiments, the polysorbate 20 concentration in the pharmaceutical composition is about 0.025% (w/v). In some embodiments, the polysorbate 20 concentration in the pharmaceutical composition is about 0.03% (w/v). In some embodiments, the polysorbate 20 concentration in the pharmaceutical composition is about 0.035% (w/v). In some embodiments, the polysorbate 20 concentration in the pharmaceutical composition is about 0.04% (w/v). In some embodiments, the polysorbate 20 concentration in the pharmaceutical composition is about 0.045% (w/v). In some embodiments, the polysorbate 20 concentration in the pharmaceutical composition is about 0.05% (w/v). In some embodiments, the polysorbate 20 concentration in the pharmaceutical composition is about 0.055% (w/v). In some embodiments, the polysorbate 20 concentration in the pharmaceutical composition is about 0.06% (w/v). In some embodiments, the polysorbate 20 concentration in the pharmaceutical composition is about 0.065% (w/v). In some embodiments, the polysorbate 20 concentration in the pharmaceutical composition is about 0.07% (w/v). In some embodiments, the polysorbate 20 concentration in the pharmaceutical composition is about 0.075% (w/v). In some embodiments, the polysorbate 20 concentration in the pharmaceutical composition is about 0.08% (w/v). In some embodiments, the polysorbate 20 concentration in the pharmaceutical composition is about 0.09% (w/v). In some embodiments, the polysorbate 20 concentration in the pharmaceutical composition is about 0.1% (w/v). In some embodiments, the polysorbate 20 concentration in the pharmaceutical composition is about 0.2% (w/v). In some embodiments, the polysorbate 20 concentration in the pharmaceutical composition is about 0.3% (w/v).

In some embodiments, the polysorbate 20 concentration in the pharmaceutical composition is about 0.03% (w/v).

In some embodiments, the non-ionic surfactant is polysorbate 80. In some embodiments, the polysorbate 80 concentration in the pharmaceutical composition is about 0.003% to 0.3% (w/v).

In some embodiments, the non-ionic surfactant is poloxamer. In some embodiments, the poloxamer concentration in the pharmaceutical composition is about 0.003% to 0.3% (w/v).

In some embodiments, the non-ionic surfactant is poloxamer 188. In some embodiments, the poloxamer 188 concentration in the pharmaceutical composition is about 0.003% to 0.3% (w/v).

In some embodiments, the non-ionic surfactant is poloxamer 407. In some embodiments, the poloxamer 407 concentration in the pharmaceutical composition is about 0.003% to 0.3% (w/v).

### Representative Embodiments/Exemplary Pharmaceutical Compositions

In some embodiments, a pharmaceutical composition is provided, the pharmaceutical composition comprising a bispecific antibody comprising a CLDN6 binding domain and a CD3 binding domain in an amount of about 1 mg/mL to about 50 mg/mL; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain, in any format as described herein, in an amount of about 5 mg/mL to about 30 mg/mL; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5.

In some embodiments, a pharmaceutical composition is provided, the pharmaceutical composition comprising a bispecific antibody comprising a CLDN6 binding domain and a CD3 binding domain in an amount of about 5 mg/mL to about 30 mg/mL; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain, in any format as described herein, in an amount of about 10 mg/mL to about 20 mg/mL; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5.

In some embodiments, a pharmaceutical composition is provided, the pharmaceutical composition comprising a bispecific antibody comprising a CLDN6 binding domain and a CD3 binding domain in an amount of about 10 mg/mL to about 20 mg/mL; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain, in any format as described herein, in an amount of about 15 mg/mL; a histidine buffer at a concentration of about 20 mM; sucrose at a concentration of about 240 mM; a polysorbate-20 in an amount of about 0.03% (w/v); and wherein the pharmaceutical composition is at a pH of about 5.9 to 6.1.

In some embodiments, a pharmaceutical composition is provided, the pharmaceutical composition comprising a bispecific antibody comprising a CLDN6 binding domain and a CD3 binding domain in an amount of about 15 mg/mL; a histidine buffer at a concentration of about 20 mM; sucrose at a concentration of about 240 mM; a polysorbate-20 in an amount of about 0.03% (w/v); and wherein the pharmaceutical composition is at a pH of about 5.9 to 6.1; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 1 mg/mL to about 50 mg/mL; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 5 mg/mL to about 30 mg/mL; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 10 mg/mL to about 20 mg/mL; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 15 mg/mL; a histidine buffer at a concentration of about 20 mM; sucrose at a concentration of about 240 mM; a polysorbate-20 in an amount of about 0.03% (w/v); and wherein the pharmaceutical composition is at a pH of about 6.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 1 mg/mL to about 50 mg/mL; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 5 mg/mL to about 30 mg/mL; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 10 mg/mL to about 20 mg/mL; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 15 mg/mL; a histidine buffer at a concentration of about 20 mM; sucrose at a concentration of about 240 mM; a polysorbate-20 in an amount of about 0.03% (w/v); and wherein the pharmaceutical composition is at a pH of about 6.0; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 1 mg/mL to about 50 mg/mL, wherein the bispecific antibody is a scFv-Fab-Fc; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 5 mg/mL to about 30 mg/mL, wherein the bispecific antibody is a scFv-Fab-Fc; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 10 mg/mL to about 20 mg/mL, wherein the bispecific antibody is a scFv-Fab-Fc; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 15 mg/mL, wherein the bispecific antibody is a scFv-Fab-Fc; a histidine buffer at a concentration of about 20 mM; sucrose at a concentration of about 240 mM; a polysorbate-20 in an amount of about 0.03% (w/v); and wherein the pharmaceutical composition is at a pH of about 6.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 1 mg/mL to about 50 mg/mL, wherein the bispecific antibody is a scFv-Fab-Fc; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 5 mg/mL to about 30 mg/mL, wherein the bispecific antibody is a scFv-Fab-Fc; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 10 mg/mL to about 20 mg/mL, wherein the bispecific antibody is a scFv-Fab-Fc; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 15 mg/mL, wherein the bispecific antibody is a scFv-Fab-Fc; a histidine buffer at a concentration of about 20 mM; sucrose at a concentration of about 240 mM; a polysorbate-20 in an amount of about 0.03% (w/v); and wherein the pharmaceutical composition is at a pH of about 6.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 1 mg/mL to about 50 mg/mL, wherein the bispecific antibody is a scFv-Fab-Fc; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 5 mg/mL to about 30 mg/mL, wherein the bispecific antibody is a scFv-Fab-Fc; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 10 mg/mL to about 20 mg/mL, wherein the bispecific antibody is a scFv-Fab-Fc; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 15 mg/mL, wherein the bispecific antibody is a scFv-Fab-Fc; a histidine buffer at a concentration of about 20 mM; sucrose at a concentration of about 240 mM; a polysorbate-20 in an amount of about 0.03% (w/v); and wherein the pharmaceutical composition is at a pH of about 6.0; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 1 mg/mL to about 50 mg/mL, wherein the bispecific antibody is an IgG-scFv format; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 5 mg/mL to about 30 mg/mL, wherein the bispecific antibody is an IgG-scFv format; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 10 mg/mL to about 20 mg/mL, wherein the bispecific antibody is an IgG-scFv format; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 15 mg/mL, wherein the bispecific antibody is an IgG-scFv format; a histidine buffer at a concentration of about 20 mM; sucrose at a concentration of about 240 mM; a polysorbate-20 in an amount of about 0.03% (w/v); and wherein the pharmaceutical composition is at a pH of about 6.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 1 mg/mL to about 50 mg/mL, wherein the bispecific antibody is an IgG-scFv format; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 5 mg/mL to about 30 mg/mL, wherein the bispecific antibody is an IgG-scFv format; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 10 mg/mL to about 20 mg/mL, wherein the bispecific antibody is an IgG-scFv format; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 15 mg/mL, wherein the bispecific antibody is an IgG-scFv format; a histidine buffer at a concentration of about 20 mM; sucrose at a concentration of about 240 mM; a polysorbate-20 in an amount of about 0.03% (w/v); and wherein the pharmaceutical composition is at a pH of about 6.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 1 mg/mL to about 50 mg/mL, wherein the bispecific antibody is an IgG-scFv format; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 5 mg/mL to about 30 mg/mL, wherein the bispecific antibody is an IgG-scFv format; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 10 mg/mL to about 20 mg/mL, wherein the bispecific antibody is an IgG-scFv format; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 15 mg/mL, wherein the bispecific antibody is an IgG-scFv format; a histidine buffer at a concentration of about 20 mM; sucrose at a concentration of about 240 mM; a polysorbate-20 in an amount of about 0.03% (w/v); and wherein the pharmaceutical composition is at a pH of about 6.0; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 1 mg/mL to about 50 mg/mL, wherein the bispecific antibody is an IgG-(scFv)₂ format; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 5 mg/mL to about 30 mg/mL, wherein the bispecific antibody is an IgG-(scFv)₂ format; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 10 mg/mL to about 20 mg/mL, wherein the bispecific antibody is an IgG-(scFv)₂ format; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 15 mg/mL, wherein the bispecific antibody is an IgG-(scFv)₂ format; a histidine buffer at a concentration of about 20 mM; sucrose at a concentration of about 240 mM; a polysorbate-20 in an amount of about 0.03% (w/v); and wherein the pharmaceutical composition is at a pH of about 6.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a pharmaceutical composition is provided, the pharmaceutical composition comprising a bispecific antibody comprising a CLDN6 binding domain and a CD3 binding domain in an amount of about 1 mg/mL to about 50 mg/mL, wherein the bispecific antibody is an IgG-(scFv)₂ format; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 5 mg/mL to about 30 mg/mL, wherein the bispecific antibody is an IgG-(scFv)₂ format; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 10 mg/mL to about 20 mg/mL, wherein the bispecific antibody is an IgG-(scFv)₂ format; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 15 mg/mL, wherein the bispecific antibody is an IgG-(scFv)₂ format; a histidine buffer at a concentration of about 20 mM; sucrose at a concentration of about 240 mM; a polysorbate-20 in an amount of about 0.03% (w/v); and wherein the pharmaceutical composition is at a pH of about 6.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, a pharmaceutical composition is provided, the pharmaceutical composition comprising a bispecific antibody comprising a CLDN6 binding domain and a CD3 binding domain in an amount of about 1 mg/mL to about 50 mg/mL, wherein the bispecific antibody is an IgG-(scFv)₂ format; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 5 mg/mL to about 30 mg/mL, wherein the bispecific antibody is an IgG-(scFv)₂ format; a histidine buffer at a concentration of about 5 mM to about 50 mM; sucrose at a concentration of about 100 mM to about 350 mM; a polysorbate-20 in an amount of about 0.003% (w/v) to about 0.1% (w/v); and wherein the pharmaceutical composition is at a pH of about 5.5 to about 6.5; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 10 mg/mL to about 20 mg/mL, wherein the bispecific antibody is an IgG-(scFv)₂ format; a histidine buffer at a concentration of about 10 mM to about 30 mM; sucrose at a concentration of about 200 mM to about 300 mM; a polysorbate-20 in an amount of about 0.01% (w/v) to about 0.05% (w/v); and wherein the pharmaceutical composition is at a pH of about 5.5 to about 6.5; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

In some embodiments, a pharmaceutical composition is provided, the pharmaceutical composition comprising a bispecific antibody comprising a CLDN6 binding domain and a CD3 binding domain in an amount of about 15 mg/mL, wherein the bispecific antibody is an IgG-(scFv)₂ format; a histidine buffer at a concentration of about 20 mM; sucrose at a concentration of about 240 mM; a polysorbate-20 in an amount of about 0.03% (w/v); and wherein the pharmaceutical composition is at a pH of about 6.0; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

### Lyophilized Compositions

In some embodiments, a lyophilized pharmaceutical composition is provided. In some embodiments, the lyophilized pharmaceutical composition is obtained via lyophilization of a pharmaceutical composition as provided for herein.

In some embodiments, the lyophilized pharmaceutical composition comprises a bispecific antibody comprising a CLDN6 binding domain and a CD3 binding domain in an amount of about 1 mg/mL to about 50 mg/mL; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, the lyophilized pharmaceutical composition comprises a bispecific antibody comprising a CLDN6 binding domain and a CD3 binding domain in an amount of about 1 mg/mL to about 50 mg/mL; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14. In some embodiments, the binding domain comprises a first VH comprising an amino acid sequence having at least 90% identity to the amino acid sequence of SEQ ID NO: 7, at least 95% identity to the amino acid sequence of SEQ ID NO: 7, or at least 98% identity to the amino acid sequence of SEQ ID NO: 7, and comprises a first VL comprising an amino acid sequence having at least 90% identity to the amino acid sequence of SEQ ID NO: 8, at least 95% identity to the amino acid sequence of SEQ ID NO: 8, or at least 98% identity to the amino acid sequence of SEQ ID NO: 8. In some embodiments, the CLDN6 binding domain comprises a first VH comprising the amino acid sequence of SEQ ID NO: 7, and comprises a first VL comprising the amino acid sequence of SEQ ID NO: 8. In some embodiments, the CD3 binding domain comprises a second VH comprising an amino acid sequence having at least 90% identity to the amino acid sequence of SEQ ID NO: 15, at least 95% identity to the amino acid sequence of SEQ ID NO: 15, or at least 98% identity to the amino acid sequence of SEQ ID NO: 15, and comprises a second VL comprising an amino acid sequence having at least 90% identity to the amino acid sequence of SEQ ID NO: 16, at least 95% identity to the amino acid sequence of SEQ ID NO: 16, or at least 98% identity to the amino acid sequence of SEQ ID NO: 16. In some embodiments, the CD3 binding domain comprises a second VH comprising the amino acid sequence of SEQ ID NO: 15, and comprises a second VL comprising the amino acid sequence of SEQ ID NO: 16.

In some embodiments, the lyophilized pharmaceutical composition comprises a bispecific antibody comprising a CLDN6 binding domain and a CD3 binding domain in an amount of about 1 mg/mL to about 50 mg/mL; a histidine buffer at a concentration of about 5 mM to about 100 mM; a sucrose at a concentration of about 50 mM to about 500 mM; a polysorbate-20 in an amount of about 0.003% (w/v) to about 0.3% (w/v); and wherein the pharmaceutical composition is at a pH of about 5.0 to about 7.0; wherein the CLDN6 binding domain comprises a heavy chain (HC) and a light chain (LC), wherein the HC comprises an amino acid sequence having at least 90% identity to the amino acid sequence of SEQ ID NO: 17, at least 95% identity to the amino acid sequence of SEQ ID NO: 17, or at least 98% identity to the amino acid sequence of SEQ ID NO: 17, and the LC comprises an amino acid sequence having at least 90% identity to the amino acid sequence of SEQ ID NO: 18, at least 95% identity to the amino acid sequence of SEQ ID NO: 18, or at least 98% identity to the amino acid sequence of SEQ ID NO: 18; and wherein the CD3 binding domain comprises an amino acid sequence having at least 90% identity to the amino acid sequence of SEQ ID NO: 19, at least 95% identity to the amino acid sequence of SEQ ID NO: 19, or at least 98% identity to the amino acid sequence of SEQ ID NO: 19. In some embodiments, the CLDN6 binding domain comprises a HC and a LC, wherein the HC comprises the amino acid sequence of SEQ ID NO: 17 and the LC comprises the amino acid sequence of SEQ ID NO: 18, and the CD3 binding domain comprises the amino acid sequence of SEQ ID NO: 19.

### Exemplary Pharmaceutical Compositions

In some non-limiting embodiments, the pharmaceutical composition comprises a composition as provided in Table 13 below:

TABLE 13
Exemplary pharmaceutical compositions

| | Protein | | | | Surfactant | | | |
|---|---|---|---|---|---|---|---|---|
| Formulation | concentration | Buffer | | Salt | (Concentration | Excipient 1 | Excipient 2 | |
| number | (mg/mL) | (Concentration) | pH | (Concentration) | (w/v)) | (Concentration) | (Concentration) | State |
| F1 | 15 | Alternative | 4.0 | — | — | — | — | Liquid |
| | | Amino acid (20 | | | | | | |
| | | mM) | | | | | | |
| F2 | 15 | Alternative | 5.0 | — | — | — | — | Liquid |
| | | Amino acid (20 | | | | | | |
| | | mM) | | | | | | |
| F3 | 15 | Citric acid (20 | 5.0 | — | — | — | — | Liquid |
| | | mM) | | | | | | |
| F4 | 15 | Citric acid (20 | 6.0 | — | — | — | — | Liquid |
| | | mM) | | | | | | |
| F5 | 15 | Histidine (20 | 6.0 | — | — | — | — | Liquid |
| | | mM) | | | | | | |
| F6 | 15 | Histidine (20 | 6.5 | — | — | — | — | Liquid |
| | | mM) | | | | | | |
| F7 | 15 | Phosphate (20 | 7.0 | — | — | — | — | Liquid |
| | | mM) | | | | | | |
| F8 | 15 | Phosphate (20 | 8.0 | — | — | — | — | Liquid |
| | | mM) | | | | | | |
| F9 | 15 | Tris (20 mM) | 8.0 | — | — | — | — | Liquid |
| F10 | 15 | Alternative | 4.0 | NaCl (150 mM) | — | — | — | Liquid |
| | | Amino acid (20 | | | | | | |
| | | mM) | | | | | | |
| F11 | 15 | Alternative | 5.0 | NaCl (150 mM) | — | — | — | Liquid |
| | | Amio acid (20 | | | | | | |
| | | mM) | | | | | | |
| F12 | 15 | Citric acid (20 | 5.0 | NaCl (150 mM) | — | — | — | Liquid |
| | | mM) | | | | | | |
| F13 | 15 | Citric acid (20 | 6.0 | NaCl (150 mM) | — | — | — | Liquid |
| | | mM) | | | | | | |
| F14 | 15 | Histidine (20 | 6.0 | NaCl (150 mM) | — | — | — | Liquid |
| | | mM) | | | | | | |
| F15 | 15 | Histidine (20 | 6.5 | NaCl (150 mM) | — | — | — | Liquid |
| | | mM) | | | | | | |
| F16 | 15 | Phosphate (20 | 7.0 | NaCl (150 mM) | — | — | — | Liquid |
| | | mM) | | | | | | |
| F17 | 15 | Phosphate (20 | 8.0 | NaCl (150 mM) | — | — | — | Liquid |
| | | mM) | | | | | | |
| F18 | 15 | Tris (20 mM) | 8.0 | NaCl (150 mM) | — | — | — | Liquid |
| F19 | 15 | Histidine (20 | 6.0 | — | Polysorbate 20 | — | — | Liquid |
| | | mM) | | | (0.01%) | | | |
| F20 | 15 | Histidine (20 | 6.0 | — | Polysorbate 20 | — | — | Liquid |
| | | mM) | | | (0.03%) | | | |
| F21 | 15 | Histidine (20 | 6.0 | — | Polysorbate 20 | — | — | Liquid |
| | | mM) | | | (0.05%) | | | |
| F22 | 15 | Histidine (20 | 6.0 | — | Polysorbate 80 | — | — | Liquid |
| | | mM) | | | (0.01%) | | | |
| F23 | 15 | Histidine (20 | 6.0 | — | Polysorbate 80 | — | — | Liquid |
| | | mM) | | | (0.03%) | | | |
| F24 | 15 | Histidine (20 | 6.0 | — | Polysorbate 80 | — | — | Liquid |
| | | mM) | | | (0.05%) | | | |
| F25 | 15 | Histidine (20 | 6.0 | — | Polysorbate 20 | Sucrose | — | Liquid |
| | | mM) | | | (0.03%) | (240 mM) | | |
| F26 | 15 | Histidine (20 | 6.0 | — | Polysorbate 20 | Sucrose | L-Methionine | Liquid |
| | | mM) | | | (0.03%) | (240 mM) | (10 mM) | |
| F27 | 15 | Histidine (20 | 6.0 | — | Polysorbate 20 | Trehalose | — | Liquid |
| | | mM) | | | (0.03%) | (240 mM) | | |
| F28 | 15 | Histidine (20 | 5.5 | — | Polysorbate 20 | Sucrose | — | Liquid |
| | | mM) | | | (0.03%) | (240 mM) | | |
| F29 | 15 | Histidine (20 | 6.5 | — | Polysorbate 20 | Sucrose | — | Liquid |
| | | mM) | | | (0.03%) | (240 mM) | | |
| F30 | 15 | Histidine (20 | 6.0 | — | Polysorbate 20 | Sucrose | — | Lyophilized |
| | | mM) | | | (0.03%) | (240 mM) | | |

In some embodiments, the pharmaceutical composition comprises a bispecific antibody, as provided for herein, in an amount of about 15 mg/mL; a citric acid buffer at a concentration of about 20 mM; and wherein the pharmaceutical composition is at a pH of about 5.0 and is a liquid.

In some embodiments, the pharmaceutical composition comprises a bispecific antibody, as provided for herein, in an amount of about 15 mg/mL; a citric acid buffer at a concentration of about 20 mM; and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; and wherein the pharmaceutical composition is at a pH of about 6.5 and is a liquid.

In some embodiments, the pharmaceutical composition comprises a bispecific antibody, as provided for herein, in an amount of about 15 mg/mL; a phosphate buffer at a concentration of about 20 mM; and wherein the pharmaceutical composition is at a pH of about 7.0 and is a liquid.

In some embodiments, the pharmaceutical composition comprises a bispecific antibody, as provided for herein, in an amount of about 15 mg/mL; a phosphate buffer at a concentration of about 20 mM; and wherein the pharmaceutical composition is at a pH of about 8.0 and is a liquid.

In some embodiments, the pharmaceutical composition comprises a bispecific antibody, as provided for herein, in an amount of about 15 mg/mL; a Tris buffer at a concentration of about 20 mM; and wherein the pharmaceutical composition is at a pH of about 8.0 and is a liquid.

In some embodiments, the pharmaceutical composition comprises a bispecific antibody, as provided for herein, in an amount of about 15 mg/mL; a citric acid buffer at a concentration of about 20 mM; a pharmaceutically acceptable form of sodium chloride present at 150 mM; and wherein the pharmaceutical composition is at a pH of about 5.0 and is a liquid.

In some embodiments, the pharmaceutical composition comprises a bispecific antibody, as provided for herein, in an amount of about 15 mg/mL; a citric acid buffer at a concentration of about 20 mM; a pharmaceutically acceptable form of sodium chloride present at 150 mM; and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; a pharmaceutically acceptable form of sodium chloride present at 150 mM; and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; a pharmaceutically acceptable form of sodium chloride present at 150 mM; and wherein the pharmaceutical composition is at a pH of about 6.5 and is a liquid.

In some embodiments, the pharmaceutical composition comprises a bispecific antibody, as provided for herein, in an amount of about 15 mg/mL; a phosphate buffer at a concentration of about 20 mM; a pharmaceutically acceptable form of sodium chloride present at 150 mM; and wherein the pharmaceutical composition is at a pH of about 7.0 and is a liquid.

In some embodiments, the pharmaceutical composition comprises a bispecific antibody, as provided for herein, in an amount of about 15 mg/mL; a phosphate buffer at a concentration of about 20 mM; a pharmaceutically acceptable form of sodium chloride present at 150 mM; and wherein the pharmaceutical composition is at a pH of about 8.0 and is a liquid.

In some embodiments, the pharmaceutical composition comprises a bispecific antibody, as provided for herein, in an amount of about 15 mg/mL; a Tris buffer at a concentration of about 20 mM; and a pharmaceutically acceptable form of sodium chloride present at 150 mM; wherein the pharmaceutical composition is at a pH of about 8.0 and is a liquid.

In some embodiments, the pharmaceutical composition comprises a bispecific antibody, as provided for herein, in an amount of about 15 mg/mL; a histidine buffer at a concentration of about 20 mM; a polysorbate-20 in an amount of about 0.01% (w/v); and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; a polysorbate-20 in an amount of about 0.03% (w/v); and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; a polysorbate-20 in an amount of about 0.05% (w/v); and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; a polysorbate-80 in an amount of about 0.01% (w/v); and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; a polysorbate-80 in an amount of about 0.03% (w/v); and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; a polysorbate-80 in an amount of about 0.05% (w/v); and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; a polysorbate-20 in an amount of about 0.03% (w/v); a sucrose at a concentration of about 240 mM; and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; a polysorbate-20 in an amount of about 0.03% (w/v); a sucrose at a concentration of about 240 mM; L-Methionine at a concentration of about 10 mM; and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; a polysorbate-20 in an amount of about 0.03% (w/v); a trehalose at a concentration of about 240 mM; and wherein the pharmaceutical composition is at a pH of about 6.0 and is a liquid.

In some embodiments, the pharmaceutical composition comprises a bispecific antibody, as provided for herein, in an amount of about 15 mg/mL; a histidine buffer at a concentration of about 20 mM; a polysorbate-20 in an amount of about 0.03% (w/v); sucrose at a concentration of about 240 mM; and wherein the pharmaceutical composition is at a pH of about 5.5 and is a liquid.

In some embodiments, the pharmaceutical composition comprises a bispecific antibody, as provided for herein, in an amount of about 15 mg/mL; a histidine buffer at a concentration of about 20 mM; a polysorbate-20 in an amount of about 0.03% (w/v); sucrose at a concentration of about 240 mM; and wherein the pharmaceutical composition is at a pH of about 6.5 and is a liquid.

In some embodiments, the pharmaceutical composition comprises a bispecific antibody, as provided for herein, in an amount of about 15 mg/mL; a histidine buffer at a concentration of about 20 mM; a polysorbate-20 in an amount of about 0.03% (w/v); sucrose at a concentration of about 240 mM; and wherein the pharmaceutical composition is at a pH of about 6.0 and is a lyophilized composition.

Although the compositions provided for herein and above may specifically reference a concentration of the antibody of about 15 mg/mL, the concentration can be adjusted upwards or downwards as determined by one of skilled in the art. For example, as provided for herein, the concentration can be about 10-20 mg/mL or about 14 to about 16 mg/mL.

In some embodiments, the composition does not comprise or contain L-methionine. In some embodiments, the composition does not comprise trehalose.

In some embodiments, the liquid compositions provided for herein can be lyophilized. In some embodiments, the lyophilized compositions are prepared by preparing the liquid composition and then lyophilizing the liquid compositions under suitable conditions to form the lyophilized composition. The lyophilized composition can be reconstituted in water.

The compositions provided for herein can be packaged in a vial, tube, or other container that can contain the composition. In some embodiments, the container is a glass vial.

In some embodiments, kits are provided comprising the compositions provided for herein. In some embodiments, the kit comprises a container comprising the composition. The kit can, in some embodiments, include one or more other elements including: instructions for use; other reagents, e.g., a label, an additional therapeutic agent, devices or other materials for preparing the therapeutic molecule for administration; pharmaceutically acceptable carriers; and devices or other materials for administration to a subject, such as a syringe.

In some embodiments, the kit comprises an auto-injector comprising the composition provided for herein.

### Methods of Treatment

In some embodiments, disclosed herein is a method for treating or preventing cancer comprising administering an effective amount of the composition of any of the preceding embodiments to a subject in need thereof. In some embodiments, the composition is administered intravenously, subcutaneously, or intramuscularly. In some embodiments, the composition is administered intravenously. In some embodiments, the composition is administered by intrathecal administration.

In some embodiments, disclosed herein is a use of the composition of any of the preceding embodiments for the preparation of a medicament for the treatment of prevention of cancer.

In some embodiments, disclosed herein is a method or use of any one of the preceding embodiments, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; glioma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; malignant rhabdoid tumor; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses; edema (e.g. that associated with brain tumors); and Meigs' syndrome.

As a non-limiting example, the prevention of an onset, the presence, and/or the evaluation of the progression of a cancer in a subject can be assessed according to the Tumor/Nodes/Metastases (TNM) system of classification (International Union Against Cancer, 6th edition, 2002), or the Whitmore-Jewett staging system (American Urological Association). Typically, cancers are staged using a combination of physical examination, blood tests, and medical imaging. If tumor tissue is obtained via biopsy or surgery, examination of the tissue under a microscope can also provide pathologic staging. In some embodiments, the stage or grade of a cancer assists a practitioner in determining the prognosis for the cancer and in selecting the appropriate modulating therapy.

In some embodiments, the prevention of an onset, or progression, of cancer is assessed using the overall stage grouping as a non-limiting example: Stage I cancers are localized to one part of the body, typically in a small area; Stage II cancers are locally advanced and have grown into nearby tissues or lymph nodes, as are Stage III cancers. Whether a cancer is designated as Stage II or Stage III can depend on the specific type of cancer. The specific criteria for Stages II and III can differ according to diagnosis. Stage IV cancers have often metastasized or spread to other organs or throughout the body. The onset or progression of cancer can be assessed using conventional methods available to one of skill in the art, such as a physical exam, blood tests, and imaging scans (e.g., X-rays, MRI, CT scans, ultrasound etc.).

As disclosed herein, administering, or administering a treatment/therapy, refers to a treatment/therapy from which a subject receives a beneficial effect, such as the reduction, decrease, attenuation, diminishment, stabilization, remission, suppression, inhibition or arrest of the development or progression of cancer, or a symptom thereof.

In some embodiments, the treatment/therapy that a subject receives, or the prevention in the onset of cancer results in at least one or more of the following effects: (1) the reduction or amelioration of the severity of cancer and/or a genetic disease or disorder, and/or a symptom associated therewith; (2) the reduction in the duration of a symptom associated with cancer and/or a genetic disease or disorder; (3) the prevention in the recurrence of a symptom associated with cancer and/or a genetic disease or disorder; (4) the regression of cancer and/or a genetic disease or disorder, and/or a symptom associated therewith; (5) the reduction in hospitalization of a subject; (6) the reduction in hospitalization length; (7) the increase in the survival of a subject; (8) the inhibition of the progression of cancer and/or a genetic disease or disorder and/or a symptom associated therewith; (9) the enhancement or improvement the therapeutic effect of another therapy; (10) a reduction or elimination in the cancer cell population, and/or a cell population associated with a genetic disease or disorder; (11) a reduction in the growth of a tumor or neoplasm; (12) a decrease in tumor size; (13) a reduction in the formation of a tumor; (14) eradication, removal, or control of primary, regional and/or metastatic cancer; (15) a decrease in the number or size of metastases; (16) a reduction in mortality; (17) an increase in cancer-free survival rate of a subject; (18) an increase in relapse-free survival; (19) an increase in the number of subjects in remission; (20) a decrease in hospitalization rate; (21) the size of the tumor is maintained and does not increase in size or increases the size of the tumor by less 5% or 10% after administration of a therapy as measured by conventional methods available to one of skill in the art, e.g., X-rays, MRI, CAT scan, ultrasound etc.; (22) the prevention of the development or onset of cancer and/or a genetic disease or disorder, and/or a symptom associated therewith; (23) an increase in the length of remission for a subject; (24) the reduction in the number of symptoms associated with cancer and/or a genetic disease or disorder; (25) an increase in symptom-free survival of a cancer subject and/or a subject associated with a genetic disease or disorder; and/or (26) limitation of or reduction in metastasis. In some embodiments, the treatment/therapy that a subject receives does not cure cancer, but prevents the progression or worsening of the disease. In some embodiments, the treatment/therapy that a subject receives does not prevent the onset/development of cancer, but may prevent the onset of cancer symptoms.

In some embodiments, disclosed herein is an isolated antibody comprising one or more of the sequences disclosed herein.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

### Enumerated Embodiments

The following examples are illustrative, but not limiting, of the compounds, compositions and methods described herein. Other suitable modifications and adaptations known to those skilled in the art are within the scope of the following embodiments.
1. A pharmaceutical composition, comprising:
   a. a bispecific antibody comprising a CLDN6 binding domain and a CD3 binding domain;
   b. a pharmaceutically acceptable buffer at a concentration of about 5 mM to about 100 mM;
   c. a sugar at a concentration of about 50 mM to about 500 mM; and
   d. a non-ionic surfactant in an amount of about 0.003% (w/v) to about 0.3% (w/v).
2. The pharmaceutical composition of embodiment 1, wherein the bispecific antibody is present at a concentration of about 1 mg/mL to about 50 mg/mL.
3. The pharmaceutical composition of embodiment 1 or embodiment 2, wherein the bispecific antibody is present at a concentration of about 5 mg/mL to about 40 mg/mL.
4. The pharmaceutical composition of any one of embodiments 1-3, wherein the bispecific antibody is present at a concentration of about 5 mg/mL to about 30 mg/mL.
5. The pharmaceutical composition of any one of embodiments 1-4, wherein the bispecific antibody is present at a concentration of about 10 mg/mL to about 25 mg/mL.
6. The pharmaceutical composition of any one of embodiments 1-5, wherein the bispecific antibody is present at a concentration of about 10 mg/mL to about 20 mg/mL or about 15 mg/mL.
7. The pharmaceutical composition of any one of embodiments 1-6, wherein the bispecific antibody is present at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 71 mg/mL, about 18 mg/mL, about 19 mg/mL, about 20 mg/mL, about 25 mg/mL, about 30 mg/mL, about 35 mg/mL, about 40 mg/mL, about 45 mg/mL, or about 50 mg/mL.
8. The pharmaceutical composition of any one of embodiments 1-7, wherein the pharmaceutically acceptable buffer is present at a concentration of about 5 mM to about 100 mM, about 5 mM to about 75 mM, about 5 mM to about 50 mM, about 10 mM to about 50 mM, about 10 mM to about 40 mM, about 15 mM to about 30 mM, or about 15 mM to about 25 mM.
9. The pharmaceutical composition of any one of embodiments 1-8, wherein the pharmaceutically acceptable buffer is histidine buffer, HEPES, phosphoric acid buffer, citric acid buffer, acetic acid buffer, succinic acid buffer, phosphate buffer, acetate buffer, citrate buffer, succinate buffer, ascorbic acid buffer, lactic acid buffer, maleic acid buffer, trometamol buffer, or gluconic acid buffer, or any combination thereof.
10. The pharmaceutical composition of any one of embodiments 1-9, wherein the pharmaceutically acceptable buffer is a histidine buffer.
11. The pharmaceutical composition of embodiment 10, wherein the histidine buffer is present at a concentration of about 10 mM to about 30 mM.
12. The pharmaceutical composition of embodiment 10 or embodiment 11, wherein the histidine buffer is present at a concentration of about 20 mM.
13. The pharmaceutical composition of any one of embodiments 1-12, wherein the sugar is present at a concentration of about 50 mM to about 500 mM, about 50 mM to about 450 mM, about 50 mM to about 400 mM, about 100 mM to about 350 mM, about 150 mM to about 350 mM, about 150 mM to about 325 mM, about 150 mM to about 300 mM, about 175 mM to about 300 mM, about 200 mM to about 300 mM, about 225 mM to about 300 mM, about 225 mM to about 275 mM, or about 230 mM to about 250 mM.
14. The pharmaceutical composition of any one of embodiments 1-13, wherein the sugar is sucrose trehalose, mannose, glucose, sorbitol, mannitol, or any combination thereof.
15. The pharmaceutical composition of any one of embodiments 1-14, wherein the sugar is sucrose.
16. The pharmaceutical composition of embodiment 15, wherein the sucrose is present at a concentration of 150 mM to 300 mM.
17. The pharmaceutical composition of embodiment 15 or 16, wherein the sucrose is present at a concentration of 200 mM to 250 mM.
18. The pharmaceutical composition of any one of embodiments 15-17, wherein the sucrose is present at a concentration of 240 mM.
19. The pharmaceutical composition of any one of embodiments 1-18, wherein the non-ionic surfactant is present in an amount of about 0.003% (w/v) to about 0.3% (w/v), about 0.003% (w/v) to about 0.2% (w/v), about 0.003% (w/v) to about 0.1% (w/v), 0.01% (w/v) to about 0.1% (w/v), 0.01% (w/v) to about 0.05% (w/v), or about 0.02% (w/v) to about 0.04% (w/v).
20. The pharmaceutical composition of any one of embodiments 1-19, wherein the non-ionic surfactant is polysorbate 20, polysorbate 80, poloxamer, poloxamer 188, or poloxamer 407.
21. The pharmaceutical composition of any one of embodiments 1-20, wherein the non-ionic surfactant is polysorbate 20.
22. The pharmaceutical composition of embodiment 21, wherein the polysorbate 20 is present in an amount of about 0.01% (w/v) to about 0.1% (w/v), 0.01% (w/v) to about 0.05% (w/v), or about 0.02% (w/v) to about 0.04% (w/v).
23. The pharmaceutical composition of embodiment 21 or embodiment 22, wherein the polysorbate 20 is present in an amount of about 0.03% (w/v).
24. The pharmaceutical composition of any one of embodiments 1-23, wherein the pharmaceutical composition has a pH of about 5.0 to about 7.5, about 5.5 to about 7, or about 5.5 to about 6.5.
25. The pharmaceutical composition of any one of embodiments 1-24, wherein the pharmaceutical composition has a pH of about 5.0, about 5.1, about 5.2, about 5.3, about
5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.
26. The pharmaceutical composition of any one of embodiments 1-25, wherein the pharmaceutical composition has a pH of about 5.9 to about 6.1.
27. A pharmaceutical composition, comprising:
   a. a bispecific antibody comprising a CLDN6 binding domain and a CD3 binding domain in an amount of about 1 mg/mL to about 50 mg/mL;
   b. a histidine buffer at a concentration of about 5 mM to about 100 mM;
   c. sucrose at a concentration of about 50 mM to about 500 mM;
   d. a polysorbate-20 in an amount of about 0.003% (w/v) to about 0.3% (w/v); and
   e. wherein the pharmaceutical composition is at a pH of about 5.0 to about 7.0.
28. A pharmaceutical composition, comprising:
   a. a bispecific antibody comprising a CLDN6 binding domain and a CD3 binding domain in an amount of about 5 mg/mL to about 30 mg/mL;
   b. a histidine buffer at a concentration of about 5 mM to about 50 mM;
   c. sucrose at a concentration of about 100 mM to about 350 mM;
   d. a polysorbate-20 in an amount of about 0.003% (w/v) to about 0.1% (w/v); and
   e. wherein the pharmaceutical composition is at a pH of about 5.5 to about 6.5.
29. A pharmaceutical composition, comprising:
   a. a bispecific antibody comprising a CLDN6 binding domain and a CD3 binding domain in an amount of about 10 mg/mL to about 20 mg/mL;
   b. a histidine buffer at a concentration of about 10 mM to about 30 mM;
   c. sucrose at a concentration of about 200 mM to about 300 mM;
   d. a polysorbate-20 in an amount of about 0.01% (w/v) to about 0.05% (w/v); and
   e. wherein the pharmaceutical composition is at a pH of about 5.5 to about 6.5.
30. A pharmaceutical composition, comprising:
   a. a bispecific antibody comprising a CLDN6 binding domain and a CD3 binding domain in an amount of about 15 mg/mL;
   b. a histidine buffer at a concentration of about 20 mM;
   c. sucrose at a concentration of about 240 mM;
   d. a polysorbate-20 in an amount of about 0.03% (w/v); and
   e. wherein the pharmaceutical composition is at a pH of about 6.0.
31. A pharmaceutical composition, comprising:
   a. A bispecific antibody comprising a CLDN6 binding domain and a CD3 binding domain in an amount of about 1 mg/mL to about 50 mg/mL, wherein the bispecific antibody is a scFv-Fab-Fc;
   b. a histidine buffer at a concentration of about 5 mM to about 100 mM;
   c. sucrose at a concentration of about 50 mM to about 500 mM;
   d. a polysorbate-20 in an amount of about 0.003% (w/v) to about 0.3% (w/v); and
   e. wherein the pharmaceutical composition is at a pH of about 5.0 to about 7.0.
32. A pharmaceutical composition, comprising:
   a. a bispecific antibody comprising a CLDN6 binding domain and a CD3 binding domain in an amount of about 5 mg/mL to about 30 mg/mL, wherein the bispecific antibody is a scFv-Fab-Fc;
   b. a histidine buffer at a concentration of about 5 mM to about 50 mM;
   c. sucrose at a concentration of about 100 mM to about 350 mM;
   d. a polysorbate-20 in an amount of about 0.003% (w/v) to about 0.1% (w/v); and
   e. wherein the pharmaceutical composition is at a pH of about 5.5 to about 6.5.
33. A pharmaceutical composition, comprising:
   a. a bispecific antibody comprising a CLDN6 binding domain and a CD3 binding domain in an amount of about 10 mg/mL to about 20 mg/mL, wherein the bispecific antibody is a scFv-Fab-Fc;
   b. a histidine buffer at a concentration of about 10 mM to about 30 mM;
   c. sucrose at a concentration of about 200 mM to about 300 mM;
   d. a polysorbate-20 in an amount of about 0.01% (w/v) to about 0.05% (w/v); and
   e. wherein the pharmaceutical composition is at a pH of about 5.5 to about 6.5.
34. A pharmaceutical composition, comprising:
   a. a bispecific antibody comprising a CLDN6 binding domain and a CD3 binding domain in an amount of about 15 mg/mL, wherein the bispecific antibody is a scFv-Fab-Fc;
   b. a histidine buffer at a concentration of about 20 mM;
   c. sucrose at a concentration of about 240 mM;
   d. a polysorbate-20 in an amount of about 0.03% (w/v); and
   e. wherein the pharmaceutical composition is at a pH of about 5.9-6.0.
35. The pharmaceutical composition of any one of embodiments 1-34, wherein the CLDN6 binding domain comprises a first variable heavy chain region (VH) and a first variable light chain region (VL), wherein the first VH comprises a HCDR1 of SEQ ID NO: 1, a HCDR2 of SEQ ID NO: 2, and a HCDR3 of SEQ ID NO: 3, and the first VL comprises a LCDR1 of SEQ ID NO: 4, a LCDR2 of SEQ ID NO: 5, and a LCDR3 of SEQ ID NO: 6.
36. The pharmaceutical composition of any one of embodiments 1-35, wherein the CLDN6 binding domain comprises a first VH comprising an amino acid sequence having at least 90% identity to the amino acid sequence of SEQ ID NO: 7, and comprises a first VL comprising an amino acid sequence having at least 90% identity to the amino acid sequence of SEQ ID NO: 8.
37. The pharmaceutical composition of any one of embodiments 1-36, wherein the CLDN6 binding domain comprises a first VH comprising an amino acid sequence having at least 95% identity to the amino acid sequence of SEQ ID NO: 7, and comprises a first VL comprising an amino acid sequence having at least 95% identity to the amino acid sequence of SEQ ID NO: 8.
38. The pharmaceutical composition of any one of embodiments 1-37, wherein the CLDN6 binding domain comprises a first VH comprising an amino acid sequence having at least 98% identity to the amino acid sequence of SEQ ID NO: 7, and comprises a first VL comprising an amino acid sequence having at least 98% identity to the amino acid sequence of SEQ ID NO: 8.
39. The pharmaceutical composition of any one of embodiments 1-38, wherein the CLDN6 binding domain comprises a first VH comprising the amino acid sequence of SEQ ID NO: 7, and comprises a first VL comprising the amino acid sequence of SEQ ID NO: 8.
40. The pharmaceutical composition of any one of embodiments 1-39, wherein the CLDN6 binding domain comprises a heavy chain (HC) and a light chain (LC), wherein the HC comprises an amino acid sequence having at least 90% identity to the amino acid sequence of SEQ ID NO: 17 and the LC comprises an amino acid sequence having at least 90% identity to the amino acid sequence of SEQ ID NO: 18.
41. The pharmaceutical composition of any one of embodiments 1-40, wherein the CLDN6 binding domain comprises a HC and a LC, wherein the HC comprises an amino acid sequence having at least 95% identity to the amino acid sequence of SEQ ID NO: 17 and the LC comprises an amino acid sequence having at least 95% identity to the amino acid sequence of SEQ ID NO: 18.
42. The pharmaceutical composition of any one of embodiments 1-41, wherein the CLDN6 binding domain comprises a HC and a LC, wherein the HC comprises an amino acid sequence having at least 98% identity to the amino acid sequence of SEQ ID NO: 17 and the LC comprises an amino acid sequence having at least 98% identity to the amino acid sequence of SEQ ID NO: 18.
43. The pharmaceutical composition of any one of embodiments 1-42, wherein the CLDN6 binding domain comprises a HC and a LC, wherein the HC comprises the amino acid sequence of SEQ ID NO: 17 and the LC comprises the amino acid sequence of SEQ ID NO: 18.
44. The pharmaceutical composition of any one of embodiments 1-43, wherein the CD3 binding domain comprises a second VH and a second VL, wherein the second VH comprises a HCDR1 of SEQ ID NO: 9, a HCDR2 of SEQ ID NO: 10, and a HCDR3 of SEQ ID NO: 11, and the second VL comprises a LCDR1 of SEQ ID NO: 12, a LCDR2 of SEQ ID NO: 13, and a LCDR3 of SEQ ID NO: 14.
45. The pharmaceutical composition of any one of embodiments 1-44, wherein the CD3 binding domain comprises a second VH comprising an amino acid sequence having at least 90% identity to the amino acid sequence of SEQ ID NO: 15, and comprises a second VL comprising an amino acid sequence having at least 90% identity to the amino acid sequence of SEQ ID NO: 16.
46. The pharmaceutical composition of any one of embodiments 1-45, wherein the CD3 binding domain comprises a second VH comprising an amino acid sequence having at least 95% identity to the amino acid sequence of SEQ ID NO: 15, and comprises a second VL comprising an amino acid sequence having at least 95% identity to the amino acid sequence of SEQ ID NO: 16.
47. The pharmaceutical composition of any one of embodiments 1-46, wherein the CD3 binding domain comprises a second VH comprising an amino acid sequence having at least 98% identity to the amino acid sequence of SEQ ID NO: 15, and comprises a second VL comprising an amino acid sequence having at least 98% identity to the amino acid sequence of SEQ ID NO: 16.
48. The pharmaceutical composition of any one of embodiments 1-47, wherein the CD3 binding domain comprises a second VH comprising the amino acid sequence of SEQ ID NO: 15, and comprises a second VL comprising the amino acid sequence of SEQ ID NO: 16.
49. The pharmaceutical composition of any one of embodiments 44-48, wherein the second VH and the second VL are linked by a polypeptide linker.
50. The pharmaceutical composition of embodiment 49, wherein the polypeptide linker comprises an amino acid sequence of SEQ ID NO: 20.
51. The pharmaceutical composition of any one of embodiments 1-50, wherein the CD3 binding domain comprises an amino acid sequence having at least 90% identity to the amino acid sequence of SEQ ID NO: 19.
52. The pharmaceutical composition of any one of embodiments 1-51, wherein the CD3 binding domain comprises an amino acid sequence having at least 95% identity to the amino acid sequence of SEQ ID NO: 19.
53. The pharmaceutical composition of any one of embodiments 1-52, wherein the CD3 binding domain comprises an amino acid sequence having at least 98% identity to the amino acid sequence of SEQ ID NO: 19.
54. The pharmaceutical composition of any one of embodiments 1-53, wherein the CD3 binding domain comprises the amino acid sequence of SEQ ID NO: 19.
55. The pharmaceutical composition of any one of embodiments 1-54, wherein the bispecific antibody comprising a CLDN6 binding domain and a CD3 binding domain is a scFv-Fab-Fc.
56. The pharmaceutical composition of any one of embodiments 1-54, wherein the bispecific antibody comprising a CLDN6 binding domain and a CD3 binding domain is an IgG-scFv.
57. The pharmaceutical composition of any one of embodiments 1-54, wherein the bispecific antibody comprising a CLDN6 binding domain and a CD3 binding domain is an IgG-(scFv)₂.
58. The pharmaceutical composition of any one of embodiments 1-34, wherein the bispecific antibody comprising a CLDN6 binding domain and a CD3 binding domain comprises:
   a first polypeptide comprising a first light chain comprising a first variable light chain region, wherein the first variable light chain region comprises:
      (1) a CDR1 sequence comprising the amino acid sequence of SEQ ID NO: 4;
      (2) a CDR2 sequence comprising the amino acid sequence of SEQ ID NO: 5; and
      (3) a CDR3 sequence comprising the amino acid sequence of SEQ ID NO: 6;
   a second polypeptide comprising a first heavy chain comprising a first variable heavy chain region and a constant domain, wherein the first variable heavy chain region comprises:
      (1) a CDR1 comprising the amino acid sequence of SEQ ID NO: 1;
      (2) a CDR2 comprising the amino acid sequence of SEQ ID NO: 2, and
      (3) a CDR3 comprising the amino acid sequence of SEQ ID NO: 3; and
   a third polypeptide comprising a second light chain and a second heavy chain and a constant domain, wherein:
   the second heavy chain comprises a second variable heavy chain region comprising:
      (1) a CDR1 comprising the amino acid sequence of SEQ ID NO: 9;
      (2) a CDR2 comprising the amino acid sequence of SEQ ID NO: 10; and
      (3) a CDR3 comprising the amino acid sequence of SEQ ID NO: 11; and
   the second light chain comprises a second variable light chain region comprising:
      (1) a CDR1 comprising the amino sequence of SEQ ID NO: 12;
      (2) a CDR2 comprising the amino acid sequence of SEQ ID NO: 13; and
      (3) a CDR3 comprising the amino acid sequence of SEQ ID NO: 14, or a variant thereof,
   wherein:
      the first polypeptide comprises an amino acid sequence that is at least 95% identical to the amino acid sequence of SEQ ID NO: 18;
      the second polypeptide comprises an amino acid sequence that is at least 95% identical to the amino acid sequence of SEQ ID NO: 17; and
      the third polypeptide comprises an amino acid sequence that is at least 95% identical to the amino acid sequence of SEQ ID NO: 19.
59. The pharmaceutical composition of embodiment 58, wherein:
the first polypeptide of the bispecific antibody comprises the amino acid sequence of SEQ ID NO: 18;
the second polypeptide of the bispecific antibody comprises the amino acid sequence of SEQ ID NO: 17; and
the third polypeptide of the bispecific antibody comprises the amino acid sequence of SEQ ID NO: 19.
60. A pharmaceutical composition, comprising:
   a. a bispecific antibody comprising a CLDN6 binding domain and a CD3 binding domain in an amount of about 1 mg/mL to about 50 mg/mL;
   b. a histidine buffer at a concentration of about 5 mM to about 100 mM;
   c. sucrose at a concentration of about 50 mM to about 500 mM;
   d. a polysorbate-20 in an amount of about 0.003% (w/v) to about 0.3% (w/v); and
   e. wherein the pharmaceutical composition is at a pH of about 5.0 to about 7.0;
wherein the CLDN6 binding domain comprises a first VH and a first VL, wherein the first VH comprises a HCDR1 of SEQ ID NO: 1, a HCDR2 of SEQ ID NO: 2, and a HCDR3 of SEQ ID NO: 3, and the first VL comprises a LCDR1 of SEQ ID NO: 4, a LCDR2 of SEQ ID NO: 5, and a LCDR3 of SEQ ID NO: 6; and
wherein the CD3 binding domain comprises a second VH and a second VL, wherein the second VH comprises a HCDR1 of SEQ ID NO: 9, a HCDR2 of SEQ ID NO: 10, and a HCDR3 of SEQ ID NO: 11, and the second VL comprises a LCDR1 of SEQ ID NO: 12, a LCDR2 of SEQ ID NO: 13, and a LCDR3 of SEQ ID NO: 14.
61. A pharmaceutical composition, comprising:
   a. a bispecific antibody comprising a CLDN6 binding domain and a CD3 binding domain in an amount of about 5 mg/mL to about 30 mg/mL;
   b. a histidine buffer at a concentration of about 5 mM to about 50 mM;
   c. sucrose at a concentration of about 100 mM to about 350 mM;
   d. a polysorbate-20 in an amount of about 0.003% (w/v) to about 0.1% (w/v); and
   e. wherein the pharmaceutical composition is at a pH of about 5.5 to about 6.5;
wherein the CLDN6 binding domain comprises a first VH and a first VL, wherein the first VH comprises a HCDR1 of SEQ ID NO: 1, a HCDR2 of SEQ ID NO: 2, and a HCDR3 of SEQ ID NO: 3, and the first VL comprises a LCDR1 of SEQ ID NO: 4, a LCDR2 of SEQ ID NO: 5, and a LCDR3 of SEQ ID NO: 6; and
wherein the CD3 binding domain comprises a second VH and a second VL, wherein the second VH comprises a HCDR1 of SEQ ID NO: 9, a HCDR2 of SEQ ID NO: 10, and a HCDR3 of SEQ ID NO: 11, and the second VL comprises a LCDR1 of SEQ ID NO: 12, a LCDR2 of SEQ ID NO: 13, and a LCDR3 of SEQ ID NO: 14.
62. A pharmaceutical composition, comprising:
   a. a bispecific antibody comprising a CLDN6 binding domain and a CD3 binding domain in an amount of about 10 mg/mL to about 20 mg/mL;
   b. a histidine buffer at a concentration of about 10 mM to about 30 mM;
   c. sucrose at a concentration of about 200 mM to about 300 mM;
   d. a polysorbate-20 in an amount of about 0.01% (w/v) to about 0.05% (w/v); and
   e. wherein the pharmaceutical composition is at a pH of about 5.5 to about 6.5;
wherein the CLDN6 binding domain comprises a first VH and a first VL, wherein the first VH comprises a HCDR1 of SEQ ID NO: 1, a HCDR2 of SEQ ID NO: 2, and a HCDR3 of SEQ ID NO: 3, and the first VL comprises a LCDR1 of SEQ ID NO: 4, a LCDR2 of SEQ ID NO: 5, and a LCDR3 of SEQ ID NO: 6; and
wherein the CD3 binding domain comprises a second VH and a second VL, wherein the second VH comprises a HCDR1 of SEQ ID NO: 9, a HCDR2 of SEQ ID NO: 10, and a HCDR3 of SEQ ID NO: 11, and the second VL comprises a LCDR1 of SEQ ID NO: 12, a LCDR2 of SEQ ID NO: 13, and a LCDR3 of SEQ ID NO: 14.
63. A pharmaceutical composition, comprising:
   a. a bispecific antibody comprising a CLDN6 binding domain and a CD3 binding domain in an amount of about 15 mg/mL;
   b. a histidine buffer at a concentration of about 20 mM;
   c. sucrose at a concentration of about 240 mM;
   d. a polysorbate-20 in an amount of about 0.03% (w/v); and
   e. wherein the pharmaceutical composition is at a pH of about 5.9-6.1;
wherein the CLDN6 binding domain comprises a first VH and a first VL, wherein the first VH comprises a HCDR1 of SEQ ID NO: 1, a HCDR2 of SEQ ID NO: 2, and a HCDR3 of SEQ ID NO: 3, and the first VL comprises a LCDR1 of SEQ ID NO: 4, a LCDR2 of SEQ ID NO: 5, and a LCDR3 of SEQ ID NO: 6; and
wherein the CD3 binding domain comprises a second VH and a second VL, wherein the second VH comprises a HCDR1 of SEQ ID NO: 9, a HCDR2 of SEQ ID NO: 10, and a HCDR3 of SEQ ID NO: 11, and the second VL comprises a LCDR1 of SEQ ID NO: 12, a LCDR2 of SEQ ID NO: 13, and a LCDR3 of SEQ ID NO: 14.
64. The pharmaceutical composition of any one of embodiments 60-63, wherein the CLDN6 binding domain comprises a first VH comprising an amino acid sequence having at least 90% identity to the amino acid sequence of SEQ ID NO: 7, and comprises a first VL comprising an amino acid sequence having at least 90% identity to the amino acid sequence of SEQ ID NO: 8.
65. The pharmaceutical composition of any one of embodiments 60-64, wherein the CLDN6 binding domain comprises a first VH comprising an amino acid sequence having at least 95% identity to the amino acid sequence of SEQ ID NO: 7, and comprises a first VL comprising an amino acid sequence having at least 95% identity to the amino acid sequence of SEQ ID NO: 8.
66. The pharmaceutical composition of any one of embodiments 60-65, wherein the CLDN6 binding domain comprises a first VH comprising an amino acid sequence having at least 98% identity to the amino acid sequence of SEQ ID NO: 7, and comprises a first VL comprising an amino acid sequence having at least 98% identity to the amino acid sequence of SEQ ID NO: 8.
67. The pharmaceutical composition of any one of embodiments 60-66, wherein the CLDN6 binding domain comprises a first VH comprising the amino acid sequence of SEQ ID NO: 7, and comprises a first VL comprising the amino acid sequence of SEQ ID NO: 8.
68. The pharmaceutical composition of any one of embodiments 60-67, wherein the CLDN6 binding domain comprises a heavy chain (HC) and a light chain (LC), wherein the HC comprises an amino acid sequence having at least 90% identity to the amino acid sequence of SEQ ID NO: 17 and the LC comprises an amino acid sequence having at least 90% identity to the amino acid sequence of SEQ ID NO: 18.
69. The pharmaceutical composition of any one of embodiments 60-68, wherein the CLDN6 binding domain comprises a HC and a LC, wherein the HC comprises an amino acid sequence having at least 95% identity to the amino acid sequence of SEQ ID NO: 17 and the LC comprises an amino acid sequence having at least 95% identity to the amino acid sequence of SEQ ID NO: 18.
70. The pharmaceutical composition of any one of embodiments 60-69, wherein the CLDN6 binding domain comprises a HC and a LC, wherein the HC comprises an amino acid sequence having at least 98% identity to the amino acid sequence of SEQ ID NO: 17 and the LC comprises an amino acid sequence having at least 98% identity to the amino acid sequence of SEQ ID NO: 18.
71. The pharmaceutical composition of any one of embodiments 60-70, wherein the CLDN6 binding domain comprises a HC and a LC, wherein the HC comprises the amino acid sequence of SEQ ID NO: 17 and the LC comprises the amino acid sequence of SEQ ID NO: 18.
72. The pharmaceutical composition of any one of embodiments 60-71, wherein the CD3 binding domain comprises a second VH comprising an amino acid sequence having at least 90% identity to the amino acid sequence of SEQ ID NO: 15, and comprises a second VL comprising an amino acid sequence having at least 90% identity to the amino acid sequence of SEQ ID NO: 16.
73. The pharmaceutical composition of any one of embodiments 60-72, wherein the CD3 binding domain comprises a second VH comprising an amino acid sequence having at least 95% identity to the amino acid sequence of SEQ ID NO: 15, and comprises a second VL comprising an amino acid sequence having at least 95% identity to the amino acid sequence of SEQ ID NO: 16.
74. The pharmaceutical composition of any one of embodiments 60-73, wherein the CD3 binding domain comprises a second VH comprising an amino acid sequence having at least 98% identity to the amino acid sequence of SEQ ID NO: 15, and comprises a second VL comprising an amino acid sequence having at least 98% identity to the amino acid sequence of SEQ ID NO: 16.
75. The pharmaceutical composition of any one of embodiments 60-74, wherein the CD3 binding domain comprises a second VH comprising the amino acid sequence of SEQ ID NO: 15, and comprises a second VL comprising the amino acid sequence of SEQ ID NO: 16.
76. The pharmaceutical composition of any one of embodiments 60-75, wherein the second VH and the second VL are linked by a polypeptide linker.
77. The pharmaceutical composition of embodiment 76, wherein the polypeptide linker comprises an amino acid sequence of SEQ ID NO: 20.
78. The pharmaceutical composition of any one of embodiments 60-77, wherein the CD3 binding domain comprises an amino acid sequence having at least 90% identity to the amino acid sequence of SEQ ID NO: 19.
79. The pharmaceutical composition of any one of embodiments 60-78, wherein the CD3 binding domain comprises an amino acid sequence having at least 95% identity to the amino acid sequence of SEQ ID NO: 19.
80. The pharmaceutical composition of any one of embodiments 60-79, wherein the CD3 binding domain comprises an amino acid sequence having at least 98% identity to the amino acid sequence of SEQ ID NO: 19.
81. The pharmaceutical composition of any one of embodiments 60-80, wherein the CD3 binding domain comprises the amino acid sequence of SEQ ID NO: 19.
82. The pharmaceutical composition of any one of embodiments 60-81, wherein the bispecific antibody comprising a CLDN6 binding domain and a CD3 binding domain is a scFv-Fab-Fc.
83. The pharmaceutical composition of any one of embodiments 60-82, wherein the bispecific antibody comprising a CLDN6 binding domain and a CD3 binding domain is an IgG-scFv.
84. The pharmaceutical composition of any one of embodiments 60-83, wherein the bispecific antibody comprising a CLDN6 binding domain and a CD3 binding domain is an IgG-(scFv)₂.
85. A pharmaceutical composition, comprising:
   a. a bispecific antibody comprising a CLDN6 binding domain and a CD3 binding domain in an amount of about 10 mg/mL to about 20 mg/mL;
   b. a histidine buffer at a concentration of about 10 mM to about 30 mM;
   c. sucrose at a concentration of about 200 mM to about 300 mM;
   d. a polysorbate-20 in an amount of about 0.01% (w/v) to about 0.05% (w/v); and
   e. wherein the pharmaceutical composition is at a pH of about 5.5 to about 6.5;
wherein the bispecific antibody comprising a CLDN6 binding domain and a CD3 binding domain comprises:
   a first polypeptide comprising a first light chain comprising a first variable light chain region, wherein the first variable light chain region comprises:
      (1) a CDR1 sequence comprising the amino acid sequence of SEQ ID NO: 4;
      (2) a CDR2 sequence comprising the amino acid sequence of SEQ ID NO: 5; and
      (3) a CDR3 sequence comprising the amino acid sequence of SEQ ID NO: 6;
   a second polypeptide comprising a first heavy chain comprising a first variable heavy chain region and a constant domain, wherein the first variable heavy chain region comprises:
      (1) a CDR1 comprising the amino acid sequence of SEQ ID NO: 1;
      (2) a CDR2 comprising the amino acid sequence of SEQ ID NO: 2, and
      (3) a CDR3 comprising the amino acid sequence of SEQ ID NO: 3; and
   a third polypeptide comprising a second light chain and a second heavy chain and a constant domain, wherein:
   the second heavy chain comprises a second variable heavy chain region comprising:
      (1) a CDR1 comprising the amino acid sequence of SEQ ID NO: 9;
      (2) a CDR2 comprising the amino acid sequence of SEQ ID NO: 10; and
      (3) a CDR3 comprising the amino acid sequence of SEQ ID NO: 11; and
   the second light chain comprises a second variable light chain region comprising:
      (1) a CDR1 comprising the amino sequence of SEQ ID NO: 12;
      (2) a CDR2 comprising the amino acid sequence of SEQ ID NO: 13; and
      (3) a CDR3 comprising the amino acid sequence of SEQ ID NO: 14, or a variant thereof,
   wherein:
      the first polypeptide comprises an amino acid sequence that is at least 95% identical to the amino acid sequence of SEQ ID NO: 18;
      the second polypeptide comprises an amino acid sequence that is at least 95% identical to the amino acid sequence of SEQ ID NO: 17; and
      the third polypeptide comprises an amino acid sequence that is at least 95% identical to the amino acid sequence of SEQ ID NO: 19.
86. A pharmaceutical composition, comprising:
   a. a bispecific antibody comprising a CLDN6 binding domain and a CD3 binding domain in an amount of about 10 mg/mL to about 20 mg/mL;
   b. a histidine buffer at a concentration of about 10 mM to about 30 mM;
   c. sucrose at a concentration of about 200 mM to about 300 mM;
   d. a polysorbate-20 in an amount of about 0.01% (w/v) to about 0.05% (w/v); and
   e. wherein the pharmaceutical composition is at a pH of about 5.5 to about 6.5;
wherein the bispecific antibody comprising a CLDN6 binding domain and a CD3 binding domain comprises:
   a first polypeptide comprising the amino acid sequence of SEQ ID NO: 18;
   a second polypeptide comprising the amino acid sequence of SEQ ID NO: 17; and
   a third polypeptide comprising the amino acid sequence of SEQ ID NO: 19.
87. A pharmaceutical composition, comprising:
   a. a bispecific antibody comprising a CLDN6 binding domain and a CD3 binding domain in an amount of about 15 mg/mL;
   b. a histidine buffer at a concentration of about 20 mM;
   c. sucrose at a concentration of about 240 mM;
   d. a polysorbate-20 in an amount of about 0.03% (w/v); and
   e. wherein the pharmaceutical composition is at a pH of about 5.9-6.1;
wherein the bispecific antibody comprising a CLDN6 binding domain and a CD3 binding domain comprises:
   a first polypeptide comprising a first light chain comprising a first variable light chain region, wherein the first variable light chain region comprises:
      (1) a CDR1 sequence comprising the amino acid sequence of SEQ ID NO: 4;
      (2) a CDR2 sequence comprising the amino acid sequence of SEQ ID NO: 5; and
      (3) a CDR3 sequence comprising the amino acid sequence of SEQ ID NO: 6;
   a second polypeptide comprising a first heavy chain comprising a first variable heavy chain region and a constant domain, wherein the first variable heavy chain region comprises:
      (1) a CDR1 comprising the amino acid sequence of SEQ ID NO: 1;
      (2) a CDR2 comprising the amino acid sequence of SEQ ID NO: 2, and
      (3) a CDR3 comprising the amino acid sequence of SEQ ID NO: 3; and
   a third polypeptide comprising a second light chain and a second heavy chain and a constant domain, wherein:
   the second heavy chain comprises a second variable heavy chain region comprising:
      (1) a CDR1 comprising the amino acid sequence of SEQ ID NO: 9;
      (2) a CDR2 comprising the amino acid sequence of SEQ ID NO: 10; and
      (3) a CDR3 comprising the amino acid sequence of SEQ ID NO: 11; and
   the second light chain comprises a second variable light chain region comprising:
      (1) a CDR1 comprising the amino sequence of SEQ ID NO: 12;
      (2) a CDR2 comprising the amino acid sequence of SEQ ID NO: 13; and
      (3) a CDR3 comprising the amino acid sequence of SEQ ID NO: 14, or a variant thereof,
wherein:
   the first polypeptide comprises an amino acid sequence that is at least 95% identical to the amino acid sequence of SEQ ID NO: 18;
   the second polypeptide comprises an amino acid sequence that is at least 95% identical to the amino acid sequence of SEQ ID NO: 17; and
   the third polypeptide comprises an amino acid sequence that is at least 95% identical to the amino acid sequence of SEQ ID NO: 19.
88. A pharmaceutical composition, comprising:
   a. a bispecific antibody comprising a CLDN6 binding domain and a CD3 binding domain in an amount of about 15 mg/mL;
   b. a histidine buffer at a concentration of about 20 mM;
   c. sucrose at a concentration of about 240 mM;
   d. a polysorbate-20 in an amount of about 0.03% (w/v); and
   e. wherein the pharmaceutical composition is at a pH of about 5.9-6.1;
wherein the bispecific antibody comprising a CLDN6 binding domain and a CD3 binding domain comprises:
   a first polypeptide comprising the amino acid sequence of SEQ ID NO: 18;
a second polypeptide comprising the amino acid sequence of SEQ ID NO: 17; and
   a third polypeptide comprising the amino acid sequence of SEQ ID NO: 19.
89. A method of treating a disease or disorder in a subject, the method comprising administering to the subject the pharmaceutical composition of any one of embodiments 1-88 to the subject thereby treating the disease or disorder.
90. The method of embodiment 89, wherein the disease or disorder is a cancer.
91. The method of embodiment 90, wherein the cancer is non-small cell lung cancer (NSCLC), ovarian cancer, gastric cancer, breast cancer, endometrial cancer, or testicular cancer.
92. The pharmaceutical composition of any one of embodiments 1-88 for use in the treatment of a disease or disorder.
93. The pharmaceutical composition of embodiment 92, wherein the disease or disorder is a cancer.
94. The pharmaceutical composition of embodiment 93, wherein the cancer is non-small cell lung cancer (NSCLC), ovarian cancer, gastric cancer, breast cancer, endometrial cancer, or testicular cancer.
95. Use of the pharmaceutical composition of any one of embodiments 1-88, for the manufacture of a medicament for treatment of a disease or disorder.
96. The use of embodiment 95, wherein the disease or disorder is a cancer.
97. The use of embodiment 96, wherein the cancer is non-small cell lung cancer (NSCLC), ovarian cancer, gastric cancer, breast cancer, endometrial cancer, or testicular cancer.

### EXAMPLES

The following examples are put forth to provide those of ordinary skill in the art with a description of how the compositions and methods described herein may be used, made, and evaluated, and are intended to be purely exemplary of the disclosure and are not intended to limit the scope of the embodiments.

### Example 1: Histidine Buffer Based Formulations are Superior to Other Buffers

A bispecific antibody that binds to CLDN6 and CD3 that is formed by the three polypeptides comprising the amino acid sequences of SEQ ID NO: 18 (Polypeptide 1), SEQ ID NO: 17 (Polypeptide 2) and SEQ ID NO: 19 (Polypeptide 3) was tested in various formulations and evaluated for various properties determined to be important for a stable and suitable drug formulation. The formulations that were tested are listed in Table 13 above and reproduced here.

| | Antibody | | | | | | | |
|---|---|---|---|---|---|---|---|---|
| | (Protein) | | | | Surfactant | | | |
| Formulation | concentration | Buffer | | Salt | (Concentration | Excipient 1 | Excipient 2 | |
| number | (mg/mL) | (Concentration) | pH | (Concentration) | (w/v)) | (Concentration) | (Concentration) | State |
| F1 | 15 | Alternative | 4.0 | — | — | — | — | Liquid |
| | | amino acid (20 | | | | | | |
| | | mM) | | | | | | |
| F2 | 15 | Alternative | 5.0 | — | — | — | — | Liquid |
| | | amino acid (20 | | | | | | |
| | | mM) | | | | | | |
| F3 | 15 | Citric acid (20 | 5.0 | — | — | — | — | Liquid |
| | | mM) | | | | | | |
| F4 | 15 | Citric acid (20 | 6.0 | — | — | — | — | Liquid |
| | | mM) | | | | | | |
| F5 | 15 | Histidine (20 | 6.0 | — | — | — | — | Liquid |
| | | mM) | | | | | | |
| F6 | 15 | Histidine (20 | 6.5 | — | — | — | — | Liquid |
| | | mM) | | | | | | |
| F7 | 15 | Phosphate (20 | 7.0 | — | — | — | — | Liquid |
| | | mM) | | | | | | |
| F8 | 15 | Phosphate (20 | 8.0 | — | — | — | — | Liquid |
| | | mM) | | | | | | |
| F9 | 15 | Tris (20 mM) | 8.0 | — | — | — | — | Liquid |
| F10 | 15 | Alternative | 4.0 | NaCl (150 mM) | — | — | — | Liquid |
| | | amino acid (20 | | | | | | |
| | | mM) | | | | | | |
| F11 | 15 | Alternative | 5.0 | NaCl (150 mM) | — | — | — | Liquid |
| | | amino acid (20 | | | | | | |
| | | mM) | | | | | | |
| F12 | 15 | Citric acid (20 | 5.0 | NaCl (150 mM) | — | — | — | Liquid |
| | | mM) | | | | | | |
| F13 | 15 | Citric acid (20 | 6.0 | NaCl (150 mM) | — | — | — | Liquid |
| | | mM) | | | | | | |
| F14 | 15 | Histidine (20 | 6.0 | NaCl (150 mM) | — | — | — | Liquid |
| | | mM) | | | | | | |
| F15 | 15 | Histidine (20 | 6.5 | NaCl (150 mM) | — | — | — | Liquid |
| | | mM) | | | | | | |
| F16 | 15 | Phosphate (20 | 7.0 | NaCl (150 mM) | — | — | — | Liquid |
| | | mM) | | | | | | |
| F17 | 15 | Phosphate (20 | 8.0 | NaCl (150 mM) | — | — | — | Liquid |
| | | mM) | | | | | | |
| F18 | 15 | Tris (20 mM) | 8.0 | NaCl (150 mM) | — | — | — | Liquid |
| F19 | 15 | Histidine (20 | 6.0 | — | Polysorbate 20 | — | — | Liquid |
| | | mM) | | | (0.01%) | | | |
| F20 | 15 | Histidine (20 | 6.0 | — | Polysorbate 20 | — | — | Liquid |
| | | mM) | | | (0.03%) | | | |
| F21 | 15 | Histidine (20 | 6.0 | — | Polysorbate 20 | — | — | Liquid |
| | | mM) | | | (0.05%) | | | |
| F22 | 15 | Histidine (20 | 6.0 | — | Polysorbate 80 | — | — | Liquid |
| | | mM) | | | (0.01%) | | | |
| F23 | 15 | Histidine (20 | 6.0 | — | Polysorbate 80 | — | — | Liquid |
| | | mM) | | | (0.03%) | | | |
| F24 | 15 | Histidine (20 | 6.0 | — | Polysorbate 80 | — | — | Liquid |
| | | mM) | | | (0.05%) | | | |
| F25 | 15 | Histidine (20 | 6.0 | — | Polysorbate 20 | Sucrose | — | Liquid |
| | | mM) | | | (0.03%) | (240 mM) | | |
| F26 | 15 | Histidine (20 | 6.0 | — | Polysorbate 20 | Sucrose | L-Methionine | Liquid |
| | | mM) | | | (0.03%) | (240 mM) | (10 mM) | |
| F27 | 15 | Histidine (20 | 6.0 | — | Polysorbate 20 | Trehalose | — | Liquid |
| | | mM) | | | (0.03%) | (240 mM) | | |
| F28 | 15 | Histidine (20 | 5.5 | — | Polysorbate 20 | Sucrose | — | Liquid |
| | | mM) | | | (0.03%) | (240 mM) | | |
| F29 | 15 | Histidine (20 | 6.5 | — | Polysorbate 20 | Sucrose | — | Liquid |
| | | mM) | | | (0.03%) | (240 mM) | | |
| F30 | 15 | Histidine (20 | 6.0 | — | Polysorbate 20 | Sucrose | — | Lyophilized |
| | | mM) | | | (0.03%) | (240 mM) | | |

The properties that were evaluated included (1) Determining the surface hydrophobicity of the of the CLDN6-CD3 bispecific antibody (e.g., the bispecific antibody as described herein) by hydrophobic interaction chromatography using a high performance liquid chromatography system (HIC-HPLC) and isoelectric point (pI) by imaged capillary isoelectric focusing (icIEF) in one standard buffer condition; (2) Investigating the colloidal and conformational stability of the CLDN6-CD3 bispecific antibody (e.g., the bispecific antibody as described herein) in a set of eighteen different buffer/pH conditions; and (3) Identifying the liabilities of the CLDN6-CD3 bispecific antibody (e.g., the bispecific antibody as described herein) with respect to oxidation stress by performing a forced methionine oxidation stress study.

The formulatability assessment and plate-based buffer/pH screen study tested eighteen formulations for optimal pH, buffer, and ionic strength (F1-F18). Five different buffer systems (ionic amino acid based, citric acid, histidine, and phosphate buffers) and a range of pH-values from 4.0 to 8.0 were evaluated with or without 150 mM NaCl. Formulations were stored at 25° C. and 40° C. for up to two weeks. Based on these stability studies, the formulation that achieved the best performance was one with histidine as the buffer at a pH of 6.0 (F5). Under these conditions, when the pH was increased to 6.5 with histidine as the buffer, the performance was not as good. The citric acid buffer formulation at pH 6.0 (F4) performed similarly to the formulation of F6, but was not as acceptable as the formulation that utilizes histidine buffer (histidine/histidine HCl) at a pH of about 6.0, which encompasses a pH from 5.90 to 6.10. Accordingly, a formulation utilizing histidine buffer without salt and at a pH of about 6.0 was selected for the subsequent surfactant screening study and formulation analysis. The specific buffer that would perform best and that salt would not be necessary for this bi-specific antibody formulation could not have been predicted in advance of performing these studies.

### Example 2: Polysorbate 20 and Polysorbate 80 can Stabilize the Antibody in a Histidine Based Buffer

Formulations based on a histidine buffer at a pH of about 6.0 were further evaluated in conjunction with surfactants. Specifically, formulations (compositions) were made with two different pharmaceutically acceptable surfactants (polysorbate 20 (PS20) or polysorbate 80 (PS80)) at three different concentrations (0.01%, 0.03%, and 0.05%) and tested under different stress conditions. These formulations are illustrated as F19-F24. The formulations were subjected to freeze/thaw stress as well as shaking to see the effect that such stresses would have on the formulations. The freeze/thaw and shaking results indicate that all formulations effectively stabilized the CLDN6-CD3 bispecific antibody against both freeze/thaw and shaking stress. Thus, histidine buffered solutions can be used with either polysorbate 20 at various concentrations as illustrated in formulations F19-F21 or polysorbate 80 at various concentrations as illustrated in F22-F24. The formulation with histidine buffer and PS20 at 0.03% was selected for the subsequent tonicity screening study.

### Example 3: Sucrose is Superior to Trehalose in Stabilizing the Antibody

The following example describes the screening of six formulations (five liquid and one lyophilized at defined pH, buffer, excipient, and surfactant concentration) under mechanical and thermal stress conditions. Specifically, the different tonicity agents, which in this case were sugars (sucrose or trehalose), were evaluated in a background formulation comprising 15 mg/mL of the bispecific antibody as provided for in Example 1, histidine buffer (20 mM) at various pH as illustrated in F25-F30, and polysorbate 20 (0.03% w/v). One formulation was tested after being lyophilized (F30). Additionally, L-Methionine (10 mM) in F26 was also evaluated for additional stabilization effects. The effect of pH on the stability of the antibody in the formulation was also evaluated by varying the pH value of ±0.5 around the target of about pH 6.0 was investigated (F28 and F29). The formulation stability study comprised mechanical (freeze/thaw and shaking) and thermal stresses (at 5° C., 25° C., and 40° C. for up to eight weeks).

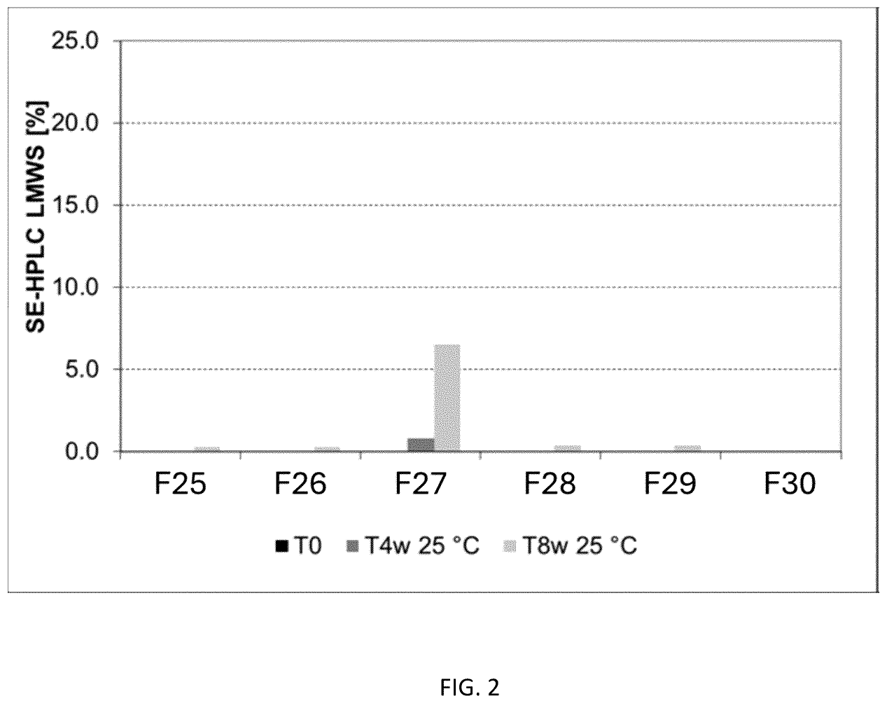

Testing by various analytical methods showed better stability of formulations containing sucrose (F25, F26, and F28-F30) than the one with trehalose (F27). Specifically, the formulation with trehalose was found to be both hazy in appearance and contain more than three particles per vial. Additionally, F27 showed a decrease in pH after eight weeks of storage as well as a change in color after 4 and 8 weeks (color value ≤BY5 to color value of →BY5 (a measured color value outside the BY scale, but closest to BY5)). The formulations with of the other liquids with sucrose remained clear and did not change color for up to 4 weeks Additionally F27 showed a significant reduction in the main purity peak by SE-HPLC after 8 weeks, indicating that the antibody was not as stable in the trehalose formulation. Specifically, there was a significant increase in low molecular weight species (LMWS) +6.5% whereas the other formulations had either no increase or only slight increases and within acceptable limits, as illustrated in FIG. 2.

This data demonstrates that not all tonicity agents, or sugars, are equivalent, and in fact, trehalose led to an unacceptable formulation as evidenced by the significant increase in LMWS. The difference between sucrose and trehalose in these formulations could not have been predicted or expected in advance.

Presence of L-methionine (F26) did not show any improved stability over eight weeks and, therefore, it was determined that the presence of L-methionine was not necessary. Variations of the pH value of ±0.5 (F28 and F29) in this background formulation also did not significantly affect drug product stability and, therefore, in conjunction with the sucrose and PS20 the acceptable pH range appears to be at least a pH of about 5.5 to about 6.5. The unreconstituted lyophilized formulation (F30) was the most stable, but can require more manipulation by the end user and, therefore, may not be as user friendly as a formulation that is a liquid when shipped.

Thus, these examples demonstrate the unexpected results of formulations that are based on a histidine buffer, as opposed to other amino acid based buffers, and those that comprise a surfactant such as PS20 or PS80 in the presence of a tonicity agent, such as sucrose. The provided for formulations (compositions) could not have been predicted or expected to have the advantages that were ultimately found.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure should not be unduly limited to such specific embodiments. Indeed, various modifications of the described modes for carrying out the disclosure are intended to be within the scope of the disclosure.

1. A pharmaceutical composition, comprising:
a. a bispecific antibody comprising a CLDN6 binding domain and a CD3 binding domain;
b. a pharmaceutically acceptable buffer at a concentration of about 5 mM to about 100 mM;
c. a sugar at a concentration of about 50 mM to about 500 mM; and
d. a non-ionic surfactant in an amount of about 0.003% (w/v) to about 0.3% (w/v).

2. The pharmaceutical composition of claim 1, wherein the bispecific antibody is present at a concentration of about 1 mg/mL to about 50 mg/mL, about 5 mg/mL to about 40 mg/mL, about 5 mg/mL to about 30 mg/mL, about 10 mg/mL to about 25 mg/mL, about 10 mg/mL to about 20 mg/mL, or about 15 mg/mL.

3.-4. (canceled)

5. The pharmaceutical composition of claim 1, wherein the pharmaceutically acceptable buffer is histidine buffer, HEPES, phosphoric acid buffer, citric acid buffer, acetic acid buffer, succinic acid buffer, phosphate buffer, acetate buffer, citrate buffer, succinate buffer, ascorbic acid buffer, lactic acid buffer, maleic acid buffer, trometamol buffer, or gluconic acid buffer, or any combination thereof.

6.-7. (canceled)

8. The pharmaceutical composition of claim 1, wherein the sugar is sucrose trehalose, mannose, glucose, sorbitol, mannitol, or any combination thereof.

9.-10. (canceled)

11. The pharmaceutical composition of claim 1, wherein the non-ionic surfactant is polysorbate 20, polysorbate 80, poloxamer, poloxamer 188, or poloxamer 407.

12. (canceled)

13. The pharmaceutical composition of claim 1, wherein the pharmaceutical composition has a pH of about 5.0 to about 7.5, about 5.5 to about 7, about 5.5 to about 6.5, or about 5.9 to about 6.1.

14.-15. (canceled)

16. The pharmaceutical composition of claim 1, comprising:
a. a bispecific antibody comprising a CLDN6 binding domain and a CD3 binding domain in an amount of about 10 mg/mL to about 20 mg/mL;
b. a histidine buffer at a concentration of about 10 mM to about 30 mM;
c. sucrose at a concentration of about 200 mM to about 300 mM;
d. a polysorbate-20 in an amount of about 0.01% (w/v) to about 0.05% (w/v); and
e. wherein the pharmaceutical composition is at a pH of about 5.5 to about 6.5.

17.-20. (canceled)

21. The pharmaceutical composition of claim 1, wherein the CLDN6 binding domain comprises a first variable heavy chain region (VH) and a first variable light chain region (VL), wherein the first VH comprises a HCDR1 of SEQ ID NO: 1, a HCDR2 of SEQ ID NO: 2, and a HCDR3 of SEQ ID NO: 3, and the first VL comprises a LCDR1 of SEQ ID NO: 4, a LCDR2 of SEQ ID NO: 5, and a LCDR3 of SEQ ID NO: 6.

22. The pharmaceutical composition of claim 1, wherein the CLDN6 binding domain comprises a first VH comprising an amino acid sequence having at least 90%, at least 95%, or at least 98%, or 100% identity to the amino acid sequence of SEQ ID NO: 7, and comprises a first VL comprising an amino acid sequence having at least 90%, at least 95%, at least 98%, or at least 100% identity to the amino acid sequence of SEQ ID NO: 8.

23. The pharmaceutical composition of claim 1, wherein the CLDN6 binding domain comprises a heavy chain (HC) and a light chain (LC), wherein the HC comprises an amino acid sequence having at least 90%, at least 95%, at least 98%, or 100% identity to the amino acid sequence of SEQ ID NO: 17 and the LC comprises an amino acid sequence having at least 90%, at least 95%, at least 98%, or 100% identity to the amino acid sequence of SEQ ID NO: 18.

24. The pharmaceutical composition of claim 1, wherein the CD3 binding domain comprises a second VH and a second VL, wherein the second VH comprises a HCDR1 of SEQ ID NO: 9, a HCDR2 of SEQ ID NO: 10, and a HCDR3 of SEQ ID NO: 11, and the second VL comprises a LCDR1 of SEQ ID NO: 12, a LCDR2 of SEQ ID NO: 13, and a LCDR3 of SEQ ID NO: 14.

25. The pharmaceutical composition of claim 1, wherein the CD3 binding domain comprises a second VH comprising an amino acid sequence having at least 90%, at least 95%, at least 98%, or 100% identity to the amino acid sequence of SEQ ID NO: 15, and comprises a second VL comprising an amino acid sequence having at least 90%, at least 95%, at least 98%, or 100% identity to the amino acid sequence of SEQ ID NO: 16.

26. The pharmaceutical composition of claim 25, wherein the second VH and the second VL are linked by a polypeptide linker, optionally wherein the polypeptide linker comprises the amino acid sequence of SEQ ID NO: 20.

27. The pharmaceutical composition of claim 1, wherein the CD3 binding domain comprises an amino acid sequence having at least 90%, at least 95%, at least 98%, or 100% identity to the amino acid sequence of SEQ ID NO: 19.

28. The pharmaceutical composition of claim 1, wherein the bispecific antibody comprising a CLDN6 binding domain and a CD3 binding domain is a scFv-Fab-Fc, an IgG-scFv, or an IgG-(scFv)₂.

29. The pharmaceutical composition of claim 16, wherein the bispecific antibody comprising a CLDN6 binding domain and a CD3 binding domain comprises:
a first polypeptide comprising a first light chain comprising a first variable light chain region, wherein the first variable light chain region comprises:
(1) a CDR1 sequence comprising the amino acid sequence of SEQ ID NO: 4;
(2) a CDR2 sequence comprising the amino acid sequence of SEQ ID NO: 5; and
(3) a CDR3 sequence comprising the amino acid sequence of SEQ ID NO: 6;
a second polypeptide comprising a first heavy chain comprising a first variable heavy chain region and a constant domain, wherein the first variable heavy chain region comprises:
(1) a CDR1 comprising the amino acid sequence of SEQ ID NO: 1;
(2) a CDR2 comprising the amino acid sequence of SEQ ID NO: 2, and
(3) a CDR3 comprising the amino acid sequence of SEQ ID NO: 3; and
a third polypeptide comprising a second light chain and a second heavy chain and a constant domain, wherein:
the second heavy chain comprises a second variable heavy chain region comprising:
(1) a CDR1 comprising the amino acid sequence of SEQ ID NO: 9;
(2) a CDR2 comprising the amino acid sequence of SEQ ID NO: 10; and
(3) a CDR3 comprising the amino acid sequence of SEQ ID NO: 11; and
the second light chain comprises a second variable light chain region comprising:
(1) a CDR1 comprising the amino sequence of SEQ ID NO: 12;
(2) a CDR2 comprising the amino acid sequence of SEQ ID NO: 13; and
(3) a CDR3 comprising the amino acid sequence of SEQ ID NO: 14, or a variant thereof,
wherein:
the first polypeptide comprises an amino acid sequence that is at least 95% identical to the amino acid sequence of SEQ ID NO: 18;
the second polypeptide comprises an amino acid sequence that is at least 95% identical to the amino acid sequence of SEQ ID NO: 17; and
the third polypeptide comprises an amino acid sequence that is at least 95% identical to the amino acid sequence of SEQ ID NO: 19.

30. The pharmaceutical composition of claim 29, wherein:
the first polypeptide of the bispecific antibody comprises the amino acid sequence of SEQ ID NO: 18;
the second polypeptide of the bispecific antibody comprises the amino acid sequence of SEQ ID NO: 17; and
the third polypeptide of the bispecific antibody comprises the amino acid sequence of SEQ ID NO: 19.

31.-38. (canceled)

39. The pharmaceutical composition of claim 16, wherein the bispecific antibody comprising a CLDN6 binding domain and a CD3 binding domain is a scFv-Fab-Fc, an IgG-scFv, or an IgG-(scFv)₂.

40.-43. (canceled)

44. A method of treating a disease or disorder in a subject, the method comprising administering to the subject the pharmaceutical composition of claim 1 to the subject thereby treating the disease or disorder.

45. (canceled)

46. The method of claim 44, wherein the cancer is non-small cell lung cancer (NSCLC), ovarian cancer, gastric cancer, breast cancer, endometrial cancer, or testicular cancer.

47.-50. (canceled)
